(19) United States
(12) Patent Application Publication
(10) Pub. No.: US 2010/0183658 A1
(43) Pub. Date: Jul. 22, 2010

(54) Novel Compounds for Enhancing MHC Class II Therapies

(75) Inventors: Kai W. Wucherpfennig, Brookline, MA (US); Melissa Joy Call, Boston, MA (US); Xuechao Xing, Wilmington, MA (US); Ross L. Stein, Cambridge, MA (US); Gregory D. Cuny, Somerville, MA (US)

Correspondence Address: FISH & RICHARDSON PC, P.O. BOX 1022, MINNEAPOLIS, MN, 55440-1022, US

(73) Assignees: The Brigham and Women's Hospital, Inc., Boston, MA (US); Dana-Farber Cancer Institute, Inc., Boston, MA (US)

(21) Appl. No.: 12/593,659

(22) PCT Filed: Mar. 28, 2008
(86) PCT No.: PCT/US08/58689

§ 371 Date: Feb. 12, 2010

Related U.S. Application Data
(60) Provisional application No. 60/920,909, filed on Mar. 30, 2007.

(51) Int. Cl. A61K 39/385; A61K 31/41; A61K 31/47; A61K 31/4439; A61K 31/433; A61K 31/427; A61K 31/506; A61K 31/497; A61K 31/405; A61K 38/02; A61P 37/06

(52) U.S. Cl. 424/193.1; 514/381; 514/313; 514/339; 514/363; 514/371; 514/275; 514/255.05; 514/419; 514/414; 514/2

(57) ABSTRACT

The invention provides classes of novel compounds that accelerate peptide loading to DR in the absence of DM and related pharmaceutical compositions. The invention also provides conjugates of these compounds with peptides, antigens or other MHC-based therapeutics, including peptides that self-catalyze their loading onto MHC Class II molecules. Methods are provided for modulating an immune response in a subject. Also disclosed are methods of using the novel compounds, e.g., in combination with MHC-based therapeutics, for the treatment of autoimmune diseases and for the manufacture of medicaments. Methods of improving loading of viral peptides and tumor peptides for enhancing the CD4 T cell response following vaccination against viruses or tumors, and related vaccines, are also provided.

### CLAIM OF PRIORITY

This application claims priority to U.S. Patent Application Ser. No. 60/920,909, filed on Mar. 30, 2007, the entire contents of which are hereby incorporated by reference.

### STATEMENT REGARDING FEDERALLY-SPONSORED RESEARCH

The invention described herein was supported, in whole or in part, by the National Institute of Health Grant No. RO1NS044914. The United States government has certain rights in the invention.

### FIELD OF THE INVENTION

The present invention relates to compounds, compositions, kits, and methods for modulating immunological responses and, more specifically, to promoting exchange of peptides on major histocompatibiltiy complex (MHC) Class II molecules.

### INTRODUCTION

The MHC-II antigen pathway offers a number of potential targets for the treatment of multiple sclerosis (MS) and other autoimmune diseases where CD4 T cells play a critical role.

The immune system consists of two components: the humoral component (antibody or B cell) and the cell-mediated immunity (T cell). T cells recognize fragments of degraded proteins or peptides (e.g., virus) and do so through specialized antigen-presenting molecules from the major histocompatibiltiy complex (MHC). These MHC molecules present either endogenous or exogenous peptides on the surface of antigen presenting cells (APC). A cell-to-cell interaction between APC and T-cell signals the T-cells to perform their immune and regulatory functions. This interaction occurs at the T cell antigen receptor (TCR) site. The TCR recognizes and transmits a signal to the interior of the T cell, resulting in the activation of T cell responses.

The MHC complex handles two types of antigens. The first type of antigen has either invaded or been taken into the APC. The APC digests these antigens into short endogenous peptide fragments and displays them on the cell surface by MHC class I proteins. The second type of antigen is derived from proteins that are ingested from the extracellular environment by phagocytosis and are endocytosed by APC. These extrinsic peptides or antigens are presented by MHC class II molecules. Whereas the MHC class I molecules present their antigens to cytotoxic T cells, MHC II molecules present antigens to helper T cells that aid B cells in generating antibody and other immune responses.

One difference in the processing of MHC class I and II molecules occurs in the endoplasmic reticulum or ER. While in the ER, MHC class II molecules are complexed to a polypeptide called the invariant chain. This complex (MHC/invariant chain) is transported through the Golgi complex to an acidic endosomal or lysosomal compartment. The complex spends a couple of hours there before reaching the cell surface. While in this compartment, the invariant chain is cleaved into small fragments, one of which is termed CLIP (class II-associated invariant peptide). The CLIP remains in the groove of the class II molecule until it is replaced by a peptide destined for presentation. The exchange of CLIP for other peptides is orchestrated by class II-related molecule called HLA-DM (DM). The DM molecule stabilizes the empty MHC class II molecules when CLIP is released and allows other peptides to associate with the MHC II class molecule. The myelin basic protein (MBP), for example, replaces the CLIP molecule and presents itself on the cell surface of the APC. In turn, the APC presents such peptides to the TCR that signals the activation of T cell responses associated with MS.

A CD4+ T cell can be differentiated into one of two subsets, Th1 or Th2. Such differentiation causes T cells to secrete a number of different cytokines and the type of cytokine secreted drives different effector pathways. Th1 cells, for example, activate macrophages and are involved in antiviral and inflammatory responses. On the other hand, Th2 cells are involved in humoral responses and allergy.

A pro-inflammatory response releases Th1 type cytokines stimulating the immune response, and in some cases results in the destruction of autologous tissue (e.g., MS). In contrast, a Th2 type response is associated with suppression of the T cell response. The Th1 and Th2 T cells use the same antigen receptor in response to an immunogen, the former producing a stimulatory response and the latter a suppressive responsive. The MHC II presentation process determines what and how long peptides are presented to the TCR. Influencing, modulating or inhibiting that process can lead to the development of disease treatments that specifically inhibits T cell activation leading to a great medical benefit.

The role of the MHC II presentation is prominent in MS. Although the etiology of MS remains unclear, the current hypothesis states that the disease develops in genetically susceptible individuals after additional environmental triggers. The strongest data suggest that one or more susceptibility genes are found on chromosomes 6p21 in the area of the major histocompatibility complex accounting for 10-60% of the genetic risk for MS. Although the role of CD4+ T cells in MS is supported directly by experimental autoimmune encephalomyelitis (EAE) animal model, there is indirect support that certain HLA class II molecules present the strongest genetic risk factor for MS, presumably via their role as antigen-presenting molecules to pathogenic CD4+ T cells.

As in other T cell-mediated autoimmune diseases, the specific genes that confer risk in MS are the HLA-DR/DQ genes and the HLA-DR15 haplotype in Caucasians (DRB1*1501, DRB5 0101, DQA1*0102, DQB1*0602). Most of the risk comes from the two DR alleles that are in very tight linkage disequilibrium. There is also a dose effect in DR15 homozygotic MS patients. Genes associated with the DR15 haplotype include transforming growth factor (TGF-β) family members, cytotoxic T lymphocyte-associated antigen-4 (CTLA-4), the tumor necrosis factor (TNF) cluster and IL-1, IL-2, IL-7m and estrogen receptors. There is no doubt that the HLA-DR (DR) and -DQ alleles and their respective molecules are by far the strongest genetic risk factors in MS.

The following mechanisms of how certain HLA class II genes confer risk for MS at the molecular level have been proposed:
1. Disease associated HLA-DR and -DQ molecules have binding characteristics that lead to preferential presentation of specific sets of self peptides, e.g., myelin peptides, in MS.
2. Disease associated HLA molecules (DR and DQ) could have binding characteristics that allow only limited sets of peptides to bind, accounting for less complete thymic negative selection of self-reactive T cells.
3. Either polymorphoric residues of the TCR (regions of DR/DQ regions or chains) select an autoimmune prone T cell repertoire.
4. Gene and protein expression of one or several disease associated DR and DQ alleles could be elevated in the CNS, enhancing antigen presentation.
5. Antigen presentation in the context of certain DR molecules could be shaped by proteases involved in antigen processing or by nonpolymorphic class II molecules such as HLA-DO and -DM to fulfill their peptide sorting and loading functions. DM has been examined but no association has been found in MS.
6. Engagement of HLA class II molecules leads to intracellular signalizing events, e.g., allergy, which could be perturbed in patents with autoimmune diseases.

In any event, the activation of genes, CD4+ autoreactive T cells, and their differentiation into Th1 and Th2 phentotypes are critical events in the initial steps and are important players in the long-term evolution of the disease.

### SUMMARY

The invention is based, inter alia, on the discovery of novel compounds that substantially accelerate loading of peptides onto MHC-II in the absence of DM. These compounds can be used to treat autoimmune disorders (e.g., multiple sclerosis, rheumatoid arthritis, or type I diabetes), to boost immunity against cancer, and to provide more potent vaccines against viruses, bacteria, and other infectious agents. Since they are able to catalyze loading across a wide pH range, these compounds can enable loading of MHC-II based therapeutics. In some embodiments, compounds described herein can be used to enable display of polypeptides of interest (e.g., cytokines) on the surface of antigen presenting cells.

A number of different therapies for autoimmune diseases require binding such therapeutics to MHC-II molecules. These compounds fall into three categories: 1. Peptides and altered peptide ligands of self-antigens that induce T cell tolerance when administered under non-inflammatory conditions, 2. Copolymers that bind to MHC-II molecules and induce the tolerogenic expansion of regulatory CD4 T cells, and 3. Inhibitors that reduce binding of self-peptides by occupying the MHC-II peptide binding groove. In most cases, such therapeutics are administrated in large doses because of proteolytic degradation and peptide competition that occurs in the late endosomal compartment where DM catalyzed peptide exchange takes place. The present invention promotes the exchange of such peptides, copolymers, and inhibitors at lower concentrations.

One aspect of the invention features compounds, as well as pharmaceutical compositions that include the compounds, represented by Structural Formula (I):

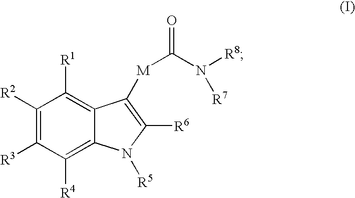

wherein M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or O, C(═X) (wherein X is NR**, O or S), OC(O), —C(═O)O, NR**, NR**CO, C(O)NR**, S(O)n′, OC(O)NR**, NR**C(O)NR**, NR** C(NR**) NR**—, and (CR**R**)n and R** independently for each occurrence, is H or lower alkyl; n is 0-5; and n′ is 0-2.

In certain embodiments, the compounds are represented by any one of Structural Formulas (Ia), (Ib), (Ic), or (Id):

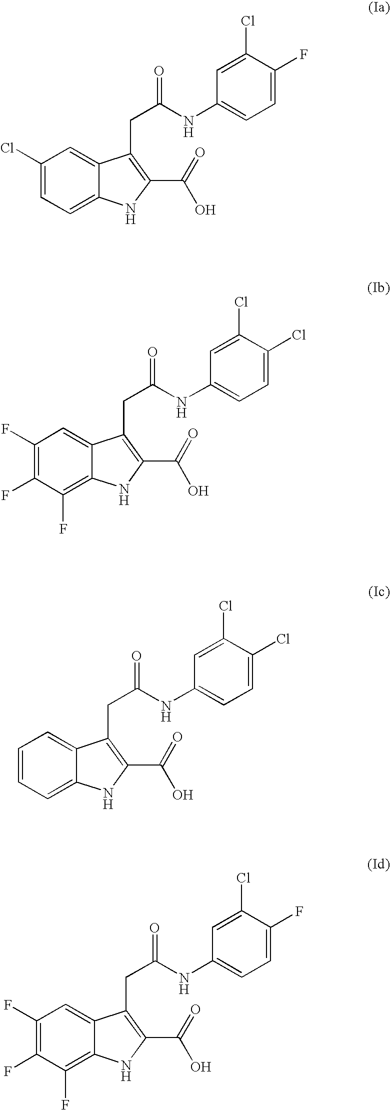

Another aspect of the invention features compounds, as well as pharmaceutical compositions that include the compounds, represented by Structural Formula (II):

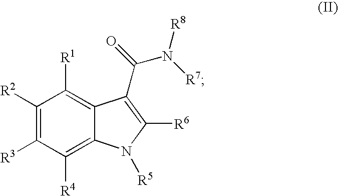

In one embodiment, the compound is represented by Structural Formula (IIa):

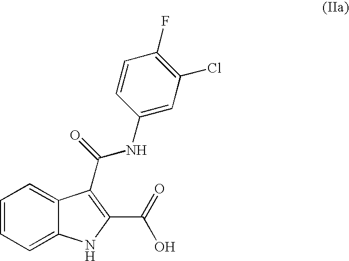

Another aspect of the invention features compounds, as well as pharmaceutical compositions that include the compounds represented by Structural Formula (III):

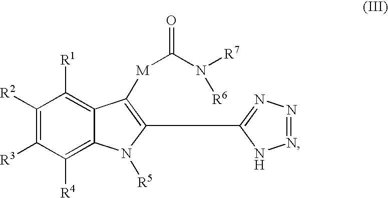

wherein M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or O, C(═X) (wherein X is NR**, O or S), OC(O), —C(═O)O, NR**, NR**CO, C(O)NR**, S(O)n′, OC(O)NR**, NR**C(O)NR**, NR** C(NR**) NR**—, and (CR**R**)n and R** independently for each occurrence, is H or lower alkyl; n is 0-5; and n′ is 0-2

In one embodiment, the compound is represented by Structural Formula (IIIa):

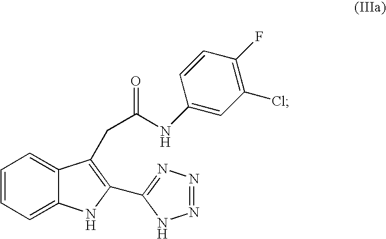

Another aspect of the invention features compounds, as well as pharmaceutical compositions that include the compounds, represented by Structural Formula (IV):

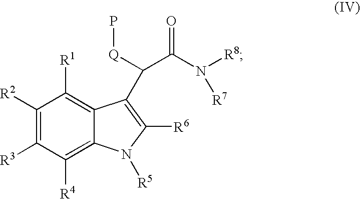

Another aspect of the invention contemplates compounds which are useful in preparing peptide conjugates, such as the compounds represented by Formula (Va), (Vb), and (Vc):

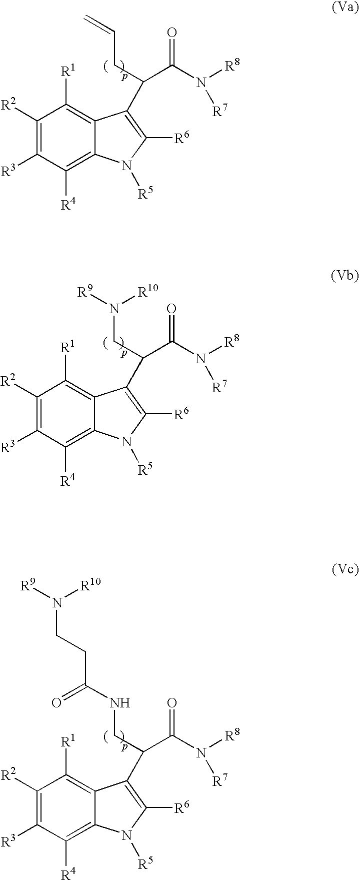

One aspect of the invention features methods of increasing exchange or loading of peptides (e.g., therapeutic peptides) on MHC Class II molecules in a subject in need thereof. The methods can include administering to the subject a therapeutically effective amount of a compound described herein (e.g., a compound represented by Structures (I), (Ia), (Ib), (Ic), (Id), (II), (IIa), (III), (IIIa), (IV), (Va), (Vb), or (Vc)). In some embodiments, the subject is afflicted with a condition that can be treated by increased CD4 T cell response. In certain embodiments, the MHC Class II molecule is HLA-DR2.

In some embodiments, the subject is afflicted with an autoimmune disorder, such as multiple sclerosis, type-I diabetes, Hashinoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barré syndrome, psoriasis, or myasthenia gravis. In certain embodiments, the autoimmune disorder is multiple sclerosis. In some embodiments, the compound is represented by formula (IV). In certain embodiments, P in formula (IV) represents a therapeutic peptide or copolymer, such as glatiramer acetate.

In some embodiments, the methods further include administering to the subject a therapeutically effective amount of a therapeutic peptide or copolymer, such as glatiramer acetate.

Exemplary therapeutic compounds (e.g., therapeutic peptides) that can be coadministered with or conjugated to compounds described herein include: (1) Peptides and altered peptide ligands of self-antigens that induce T cell tolerance when administered under non-inflammatory conditions, (2) Copolymers that bind to MHC-II and induce the tolerogenic expansion of regulatory CD4 T cells, and (3) Inhibitors that reduce binding of self-peptides by occupying the MHC-II peptide binding groove. Typically, these therapeutics are administrated in large doses because of proteolytic degradation and peptide competition in the late endosomal compartment where DM catalyzed peptide exchange takes place. The compounds described herein can improve the efficacy of these MHC-II based therapeutics, by providing these therapeutic compounds access to a larger pool of MHC-II and reducing competition by peptides generated by proteolysis in the late endosome.

In another aspect, the invention features methods of treating an autoimmune disorder in a subject that include administering to the subject a therapeutically-effective amount of a compound described herein (e.g., a compound represented by Structures (I), (Ia), (Ib), (Ic), (Id), (II), (IIa), (III), (IIIa), (IV), (Va), (Vb), or (Vc)). In some embodiments, the methods further include administering to the subject a therapeutic compound (e.g., a therapeutic peptide).

In another aspect, the invention features methods of administering therapeutic peptides to a subject that include administering to the subject a compound described herein (e.g., a compound represented by Structures (I), (Ia), (Ib), (Ic), (Id), (II), (IIa), (III), (IIIa), (IV), (Va), (Vb), or (Vc)). In some embodiments, the therapeutic peptide is conjugated to the compound (e.g., at the N- or C-terminus). In some embodiments, the therapeutic peptide and the compound are co-administered to the subject. In some embodiments, the subject is afflicted with a condition that can be treated by increased CD4 T cell response. In certain embodiments, the MHC Class II molecule is HLA-DR2. In some embodiments, the methods allow for a reduction in the amount of the therapeutic peptide as compared to administration of the therapeutic peptide alone.

In another aspect, the invention features methods of displaying polypeptides on the surface of antigen presenting cells (e.g., that express MHC II), by administering to the cells a polypeptide linked to an MHC-binding peptide and a compound described herein (e.g., a compound represented by Structures (I), (Ia), (Ib), (Ic), (Id), (II), (IIa), (III), (IIIa), (IV), (Va), (Vb), or (Vc)). In some embodiments, the polypeptide is a cytokine.

In another aspect, the invention features compounds described herein (e.g., compounds represented by Structures (I), (Ia), (Ib), (Ic), (Id), (II), (IIa), (III), (IIIa), (IV), (Va), (Vb), or (Vc)) for use as a medicament.

In other aspects, the invention features the use of compounds described herein (e.g., compounds represented by Structures (I), (Ia), (Ib), (Ic), (Id), (II), (IIa), (III), (IIIa), (IV), (Va), (Vb), or (Vc)) for the preparation of a medicament for the treatment of autoimmune disorders (e.g., multiple sclerosis, rheumatoid arthritis, or type I diabetes) or cancers, or for the preparation of a vaccine composition against viruses, bacteria and other infectious agents.

One aspect of the invention features kits that include the compounds described herein (e.g., compounds represented by Structures (I), (Ia), (Ib), (Ic), (Id), (II), (IIa), (III), (IIIa), (IV), (Va), (Vb), or (Vc)). One aspect features kits that include: (i) a first container that contains a pharmaceutical composition that includes any one of the compounds disclosed herein; and (ii) second container that contains an antigen. In one embodiment, the antigen is a cancer antigen.

In one embodiment, the antigen is a viral antigen, a bacterial antigen, a fungal antigen or a parasitic antigen.

The present invention provides compositions and methods to promote the binding of peptides to DR molecules and substantially reduce the dose of peptide required for an equivalent level of presentation (˜10-fold). Such DR molecules can be used as a display platform for immunomodulatory molecules. Given that high-affinity peptides have long half-lives on DR molecules on the cell surface, the present invention provides DR-bound peptides as anchors for long-lived display of therapeutic peptides or cytokines on the cell surface. When T cells migrate through secondary lymphoid structures they will form stable interactions in the presence of the invention. These interactions last for many hours giving the APC an opportunity to present either a specific MHC-peptide or MHC-cytokine complex. These complexes are recognized by the TCR where the display of peptides or cytokines via MHC class II molecules concentrates these peptides or cytokines and influences T cell differentiation. The peptides or cytokines presented at that site determine the differentiation of T cells into subsets with either an effector (Th1) or regulatory (Th2) phenotype. The present invention improves the efficacy of peptides or cytokines that down-modulate chronic inflammatory responses and modulates immune responses in a variety of situations, including autoimmune diseases, allergic diseases and organ transplantation. This “self-catalyzed loading” concept can also be used to enhance T cell responses to induce differentiation of long-lived memory T cells with effector properties (e.g., IL-15).

The new compounds and methods improve the efficacy of the above three classes of MHC-II based therapeutics. They can catalyze loading at these sites, provide access to a larger pool of MHC-II molecules, and reduce peptide competition generated by proteolysis in the late endosome. This approach has broad applicability for therapeutics to human autoimmune diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A is a schematic representation of MHC class II loading and exchange pathways and novel sites of peptide loading through the action of Compound (Ia). Compound (Ia) is active on MHC class II molecules even at a neutral and slightly acidic pH, enabling loading of MHC class II molecules at sites that lack the natural exchange catalyst DM.

FIG. 1B is a schematic representation of an example of use of a compound described herein (Compound) for targeting of cytokines to the surface of MHC class II expressing antigen presenting cells. By conjugating the compounds (Compound) to a composition that includes a MHC class II binding peptide (Peptide) and a cytokine (Cytokine), the peptides are loaded onto DR at the surface of antigen presenting cells for long-lived display at the cell surface.

FIG. 1C is a schematic representation of soluble DR molecules with a covalently linked CLIP peptide.

FIG. 1D is an electrophoresis gel that shows the expression of four different DR molecules. SDS-PAGE demonstrated that these protein preparations were pure and that the linker could be cleaved with thrombin (reduced MW of the DRγ chain following cleavage; lower MW band on SDS-PAGE).

FIG. 2 is a graph showing the detection of DR2 binding to labeled myelin basic protein (MBP) peptide by fluorescence polarization. This experiment demonstrates that fluorescence polarization (FP) increases as a larger fraction of the labeled peptide becomes receptor-bound. The Alexa™-488 labeled MBP peptide was used at a concentration of 10 nM and increasing quantities of thrombin-cleaved DR2/CLIP were added to reactions (40 μl volume, 384-well plate). FP values were determined following overnight incubation at 37° C.

FIG. 3 is a graph showing real-time analysis of DM-catalyzed peptide exchange by fluorescence polarization.

FIG. 4A is a graph showing peptide exchange of DR/CLIP complexes and Alexa™-488 labeled MBP peptide incubated at pH 5.2 without Compound (Ia) or increasing concentrations of Compound (Ia).

FIG. 4B is a graph showing acceleration of dissociation of Alexa™-488 labeled MBP peptide in the presence of Compound (Ia) as compared to the absence of Compound (Ia) (DMSO control).

FIG. 5 is a graph showing acceleration of the rate of peptide association to empty DR2 molecules by Compound (Ia).

FIG. 6 is a bar graph showing the relationship between Compound (Ia) activity and pH. Compound (Ia) is active over a wide pH range, with maximum activity detected at pH 5.25.

FIG. 7 is a histogram showing that Compound (Ia) increases the presentation of MBP on MGAR cells.

FIG. 8 is a schematic representation of the self-catalyzed loading of peptide through a linked small molecule with DM-like catalytic function.

FIG. 9A is a schematic representation of the structure of Compound (Ia) with a linker.

FIG. 9B is a graph demonstrating that the Compound (Ia)-linker molecule is as potent as Compound (Ia) without the linker.

FIG. 10 is a schematic of the synthesis of a Compound (Ia)-maleimide derivative.

FIG. 11 is a graph demonstrating enhancement of self-catalyzed peptide loading by MBP-Compound (Ia) conjugates.

FIG. 12A is a graph showing competition of MBP and MBP conjugated to Compound (Ia) at either the N- or C-terminus for binding to DR/CLIP.

FIG. 12B is a graph showing IL-2 released from T cell hybridomas in the presence of MGAR cells loaded with MBP (85-99) peptide, MBP peptide in the presence of Compound (Ia), and MBP peptide conjugated to Compound (Ia) at either the N- or C-terminus.

FIG. 13A is a representation of the structure of Compound (Ib).

FIG. 13B is a graph showing the activity of Compound (Ia) and Compound (Ib) in catalyzing loading of the MBP peptide to DR2.

FIG. 14A is a representation of the structures of Compounds (Ic) and (Id).

FIG. 14B is a graph depicting the activity of Compounds (Ia), (Ib), (Ic), and (Id) in catalyzing loading of the MBP peptide to DR2.

### DETAILED DESCRIPTION

### I. Overview

Applicants have discovered, inter alia, families of small molecules that substantially accelerate the loading of peptides onto MHC class II molecules. Without limiting the scope of the invention, these compounds have broad therapeutic utility in any application requiring a more efficient induction of a CD4 T cell response, including enhancing the efficacy of MHC class II based therapeutics in the treatment of autoimmune diseases (e.g., multiple sclerosis, rheumatoid arthritis, or type I diabetes), infectious agents, and cancer. These compounds can be conjugated to peptides to allow autocatalysis of peptide loading.

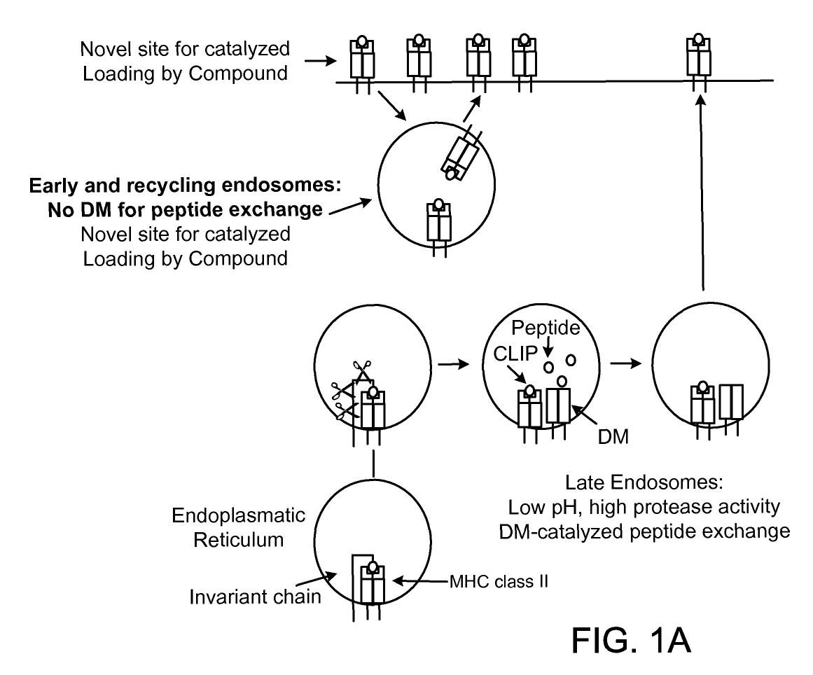

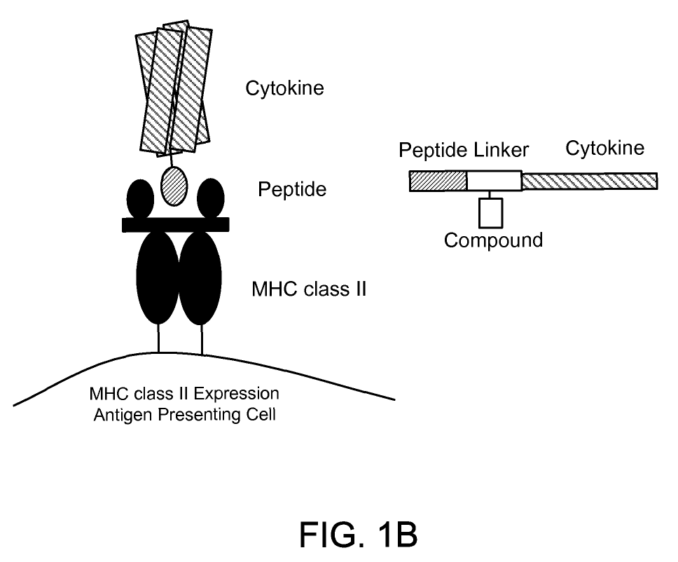

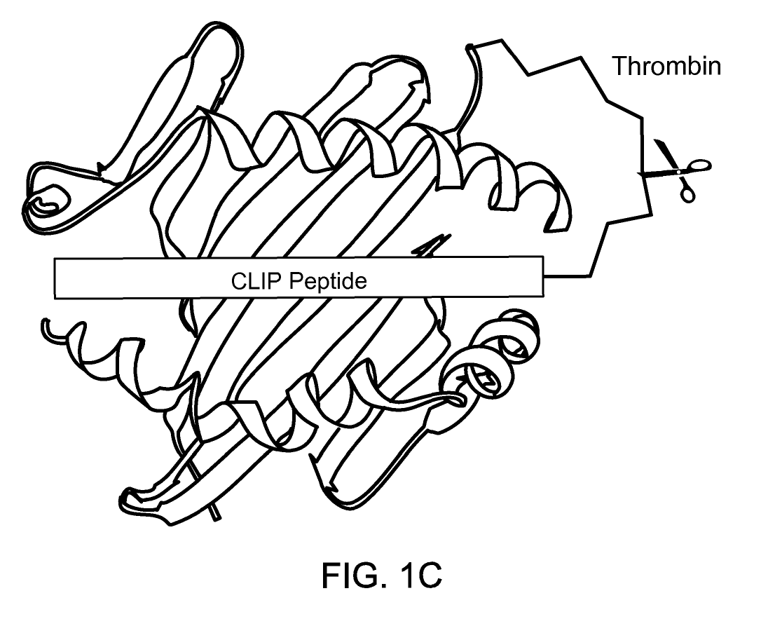

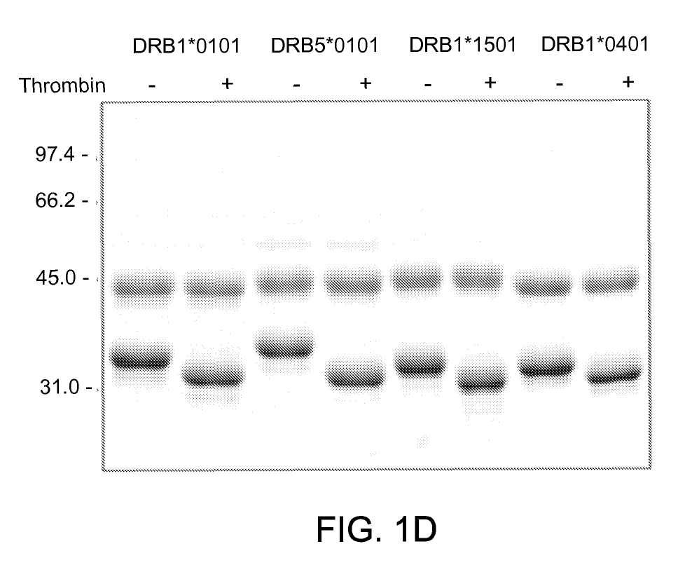

Nascent MHC class II molecules (MHC-II) assemble in the endoplasmic reticulum into a complex composed of an invariant chain timer and three MHC-II molecules (e.g., DR molecule). The CLIP segment of the invariant chain protects the hydrophobic peptide-binding groove of the MHC-II molecule. The N-terminal cytoplasmic domain contains a targeting motif that directs transport of MHC-II-invariant chain complexes to endosomes (FIG. 1A). In the endosomal/lysosomal compartment, the invariant chain is cleaved by several proteases in a stepwise fashion. These proteolytic steps trim the invariant chain down to the CLIP segment that remains bound in the peptide-binding groove. The exchange of CLIP with peptides from exogenous antigens supplied by the endocytic pathway is catalyzed in a late endosomal compartment by the HLA-DM (DM) enzyme. The stability of peptides for the MHC-II binding site is pH dependent and such complexes have high stability at neutral pH at the cell surface.

One aspect of the invention features small molecules that enable display of therapeutics at the cell surface following binding of a linked peptide either at the cell surface or in slightly acidic early endosomes in the recycling pathway.

The invention features compounds that enhance peptide exchange of MHC class II molecules. The invention also features compositions, and in particular pharmaceutical compositions, that include such compounds. In some embodiments, the compositions include a peptide or peptidomimetic product, capable of binding to an MHC class II molecule. In some embodiments, the compound is conjugated to the peptide or peptidomimetic, such that the conjugate autocatalyzes its loading onto an MHC class II molecule. The compounds can be conjugated at the N-terminus, at the C-terminus, or internally, or a combination thereof. In one embodiment, the peptide self-catalyzes its loading by conjugation of the compound at or near its C-terminus.

Another aspect of the invention features methods for treating a subject afflicted with or at risk of developing an autoimmune disorder. In some embodiments, the methods include administering to the subject (i) one of the compounds disclosed herein having DM-like activity, and (ii) a peptide or peptidomimetic capable of binding to an MHC Class II molecule. In certain embodiments, the disorder is Multiple Sclerosis (MS). In some embodiments, the peptide includes myelin basic protein (MBP), proteolipid protein (PLP), myelin-associated glycoprotein (MAG), or myelin olgiodendrocyte glycoprotein (MOG). The suppression of the T cell responsiveness to these antigens can be used to inhibit or treat demyelinating diseases. In certain embodiments, the disorder is MS and the peptide is glatiramer acetate. The compound can be conjugated to the peptide (e.g., glatiramer acetate) or it can be administered separately or both. In certain embodiments the disorder is insulin-dependent diabetes mellitus (IDDM), which is a disease characterized by autoimmune destruction of the beta cells in the pancreatic islet of Langerhans. In some embodiments, the peptide includes an epitope of insulin or glutamic acid decarboxylase (GAD).

Another aspect of the invention features methods of enhancing MHC Class II catalyzed peptide exchange. In some embodiments, the methods include administering a composition that includes one or more of the compounds having DM catalytic activity. The methods can be practiced in vitro, ex vivo, or in vivo. In certain embodiments, the MHC Class II molecule is HLA-DR2. In various embodiments, the cell is a dendritic cell, a macrophage, a CD-40 activated B cell, or another professional antigen presenting cell. In various embodiments, the peptide is an autoantigen, a cancer antigen, a bacterial antigen, a viral antigen, a parasitic antigen or a fungal antigen.

Another aspect of the invention features methods for treating a subject having, or at risk of having, cancer. In some embodiments embodiment, the method includes administering a composition that includes one or more of the compounds having DM catalytic activity. In some embodiments, a cancer antigen is also administered to the subject. The compounds can be conjugated to the peptide or they can be administered separately. In certain embodiments, the cancer expresses a cancer antigen. In various embodiments, the cancer is a leukemia, a melanoma, a renal cell carcinoma, a colon cancer, a liver cancer, a pancreatic cancer, or a lung cancer. In other embodiments, the cancer expresses MHC class II molecules. In certain embodiments, the cancer is a B-cell lymphoma. In some embodiments, the cancer is a refractory cancer. In various embodiments, the subject has had or is scheduled to have surgery, radiation treatment or chemotherapy to treat the cancer. In some embodiments, the methods include administering to the subject an anti-cancer agent. The anti-cancer agent can be, for example, a cytotoxic agent or an antibody.

Another aspect of the invention features methods for treating a subject having, or at risk of having, an infectious disease. In some embodiments, the methods include administering a composition that includes one or more of the compounds having DM catalytic activity. The infectious disease can be, for example, a viral infection, a bacterial infection, a fungal infection or a parasitic infection. In some embodiments, the infectious disease is a chronic infection. In various embodiments, the infectious disease is a chronic infection with HIV, Hepatitis C or tuberculosis.

In some embodiments, the subject has a bacterial infection and the method further includes administering to the subject an anti-bacterial agent. In other embodiments, the subject has a viral infection and the method further includes administering to the subject an anti-viral agent. In other embodiments, the subject has a fungal infection and the method further includes administering to the subject an anti-fungal agent. In certain embodiments, the subject has a parasitic infection and the method further includes administering to the subject an anti-parasitic agent.

In some embodiments, the methods further involve administering to the subject a pathogen antigen. The antigen can be, for example, a viral antigen, a bacterial antigen, a fungal antigen, or a parasitic antigen. In certain embodiments, the invention further features administering to the subject one or more immunomodulatory agents, with or without the antigen. Examples of immunomodulatory agents are an adjuvant, a hematopoietic cell stimulator, a cytokine, a growth factor and an immunostimulatory oligonucleotide.

Another aspect of the invention features methods for preparing cells. In some embodiment, the method involves administering to a subject (i) a compound that promotes MHC Class II peptide exchange, and optionally (ii) a peptide or peptidomimetic capable of binding to an MHC Class II molecule. In certain embodiments, the immune system cells obtained are T cells. In various embodiments, the immune system cells are dendritic cells, macrophages, CD-40 activated B cells, or professional antigen presenting cells. In some embodiments, the subject has an infectious disease.

In certain embodiments of the methods described herein, one or more immunomodulatory agents are also administered to the subject. Examples of immunomodulatory agents include adjuvants, a hematopoietic cell stimulator, cytokines, growth factors or immunostimulatory oligonucleotides.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “inhibiting” is art-recognized, and when used in relation to a condition, such as a local recurrence (e.g., pain), a disease such as cancer, a syndrome complex such as heart failure or any other medical condition, is well understood in the art, and includes administering, prior to onset of the condition, a composition that reduces the frequency of, reduces the severity of, prevents, or delays the onset of symptoms of a medical condition in a subject relative to a subject which does not receive the composition. Thus, inhibition (e.g., prevention) of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Inhibition (e.g., prevention) of an infection includes, for example, reducing the number of diagnoses of the infection in a treated population versus an untreated control population, and/or delaying the onset of symptoms of the infection in a treated population versus an untreated control population.

The term “effective amount” as used herein is defined as an amount effective, at dosages and for periods of time necessary to achieve a desired result. The effective amount of a compound described herein can vary according to factors such as the disease state, age, sex, and weight of the subject. Dosage regimens can be adjusted to provide the optimum therapeutic response. For example, several divided doses can be administered daily or the dose can be proportionally reduced as indicated by the exigencies of the therapeutic situation.

A “therapeutically effective amount” of a compound with respect to the subject method of treatment, refers to an amount of the compound(s) in a preparation which, when administered as part of a desired dosage regimen (to a mammal, e.g., a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

A “subject” as used herein refers to any vertebrate animal, e.g., a primate or mammal, such as a human. Examples of subjects include humans, non-human primates, rodents, guinea pigs, rabbits, sheep, pigs, goats, cows, horses, dogs, cats, birds, and fish.

The term “acyl” is art-recognized and refers to a group represented by the general formula hydrocarbylC(O)—, preferably alkylC(O)—.

The term “acylamino” is art-recognized and refers to an amino group substituted with an acyl group and can be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and can be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. In some embodiments, the alkenyl group is a C₂₋₆ alkenyl group. Such substituents can occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In some embodiments, the alkyl group is C₁₋₄ alkyl or C₁₋₆ alkyl. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl can include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and can be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents can occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

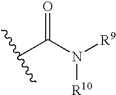

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. In some embodiments, R9 and R10 are selected from H and C₁₋₆ alkyl.

The terms “amine” and “amino” are art-recognized and refer to both unsubstituted and substituted amines and salts thereof, e.g., a moiety that can be represented by

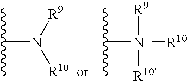

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “amino protecting group” refers to any group attached to an amine intended to protect that amine from inadvertent reactivity. Example amino protecting groups include —C(O)—OBz (CBz), —C(O)—O-t-Bu (Boc), Fmoc, acyl, benzyl, and the like. An example protected amine is maleimide.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

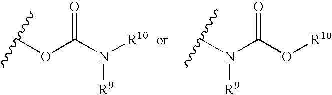

wherein R⁹ and R¹⁰ independently represent hydrogen or a hydrocarbyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group can be hydrocarbyl-O—. Ethers can be either symmetrical or unsymmetrical. Examples of ethers include, but are not limited to, heterocycle-O-heterocycle and aryl-O-heterocycle. Ethers include “alkoxyalkyl” groups, which can be represented by the general formula alkyl-O-alkyl.

The terms “halo” and “halogen” as used herein means halogen and includes chloro, fluoro, bromo, and judo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g.; the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hetcrocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but can optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “aromatic” refers to optionally substituted aryl or heteroaryl.

The term “aliphatic” refers to optionally substituted groups that are not aromatic. These include optionally substituted alkyl, akenyl, alkynyl, cycloalkyl, heteroalkyl, heterocycles, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen can have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “sulfate” is art-recognized and refers to the group —OSO₃H, or a pharmaceutically acceptable salt thereof.

The term “sulfonamide” is art-recognized and refers to the group represented by the general formulae

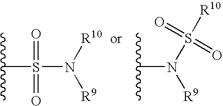

wherein R⁹ and R¹⁰ independently represents hydrogen or hydrocarbyl.

The term “sulfoxide” is art-recognized and refers to the group —S(O)—R⁹, wherein R⁹ represents a hydrocarbyl.

The term “sulfonate” is art-recognized and refers to the group —SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and can be represented by the general formula

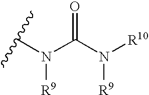

wherein R⁹ and R¹⁰ independently represent hydrogen or a hydrocarbyl.

The term “cancer cells” as used herein includes all cells of forms of cancer or neoplastic disease.

The term “a cell” as used herein includes a plurality of cells. Administering a compound to a cell includes in vivo, ex vivo, and in vitro administration.

To “inhibit” or “suppress” or “reduce” a function or activity, such as cancer cell proliferation, is to reduce the function or activity when compared to otherwise same conditions except for a condition or parameter of interest, or alternatively, as compared to another conditions.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material useful for formulating a drug for medicinal or therapeutic use. Each carrier must be “acceptable” in the sense of being compatible with other ingredients of the formulation and not injurious to the patient.

Some examples of materials that can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts can exist in either a hydrated, solvated or substantially anhydrous form. In general, the acid addition salts of compounds of any of Formulas I-V can be more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, can be used, for example, in the isolation of compounds of any of Formulas I-V for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by any of Formulas I-VI or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “solvate” as used herein means a compound of any of Formulas I-V, or a pharmaceutically acceptable salt of a compound of any of Formulas I-V, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, inhibiting spread of disease, delay or slowing of disease progression, amelioration or palliation of the disease state, and remission (whether partial or total), whether detectable or undetectable.

“Treatment” can also mean prolonging survival as compared to expected survival if not receiving treatment.

At various places in the present specification, substituents of compounds described herein are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as □-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein further include hydrates and solvates, as well as anhydrous and non-solvated forms.

The term, “compound,” as used herein is meant to include all stereoisomers, geometric iosomers, tautomers, and isotopes of the structures depicted.

All compounds, and pharmaceutically acceptable salts thereof, can be found together with other substances such as water and solvents (e.g. hydrates and solvates) or can be isolated.

Compounds described herein can also include all isotopes of atoms occurring in the intermediates or final compounds. Isotopes include those atoms having the same atomic number but different mass numbers. For example, isotopes of hydrogen include tritium and deuterium.

In some embodiments, the compounds described herein, and salts thereof, are substantially isolated. By “substantially isolated” is meant that the compound is at least partially or substantially separated from the environment in which is was formed or detected. Partial separation can include, for example, a composition enriched in the compound described herein. Substantial separation can include compositions containing at least about 50%, at least about 60%, at least about 70%, at least about 80%, at least about 90%, at least about 95%, at least about 97%, or at least about 99% by weight of the compound described herein, or salt thereof. Methods for isolating compounds and their salts are routine in the art.

### II. Compounds

One aspect of the invention features compounds, as well as pharmaceutical compositions that includes the compounds, represented by Structural Formula (I):

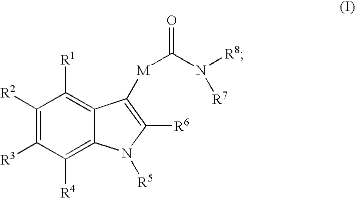

wherein: R¹, R², R³, and R⁴ are each independently selected from —H, —Cl, —F, —CH₃, —Br, —CF₃, —OCF₃, —CN, —CO₂R*, —OR*, —NR*R*, —SO₂R*, and —SO₂NR*R*; R* in each occurrence is independently selected from H, and substituted or unsubstituted alkyl, aryl and alkenyl; R⁵ is —H, -lower alkyl, or lower alkenyl, R⁶ is —CO₂H, —CO₂R′, —SO₃H or SO₃R′; R′ is lower alkyl; R⁷ is aromatic, aliphatic, or alkyl interrupted by one or more heteroatoms; R⁸ is —H or —CH₃ and M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, NR*—C(NR**)—NR**—, and —(CR**R**)ₙ— and R^{**} independently for each occurrence, is H or lower alkyl; n is 0-5; and n′ is 0-2.

In one embodiment, the R⁷ of the compound is a substituted phenyl group. In one embodiment, R⁷ of the compound is a 3,4-dihalo substituted phenyl group where the halogens are independently selected from —Br, —Cl or —F. In one embodiment, R⁷ of the compound is selected from:

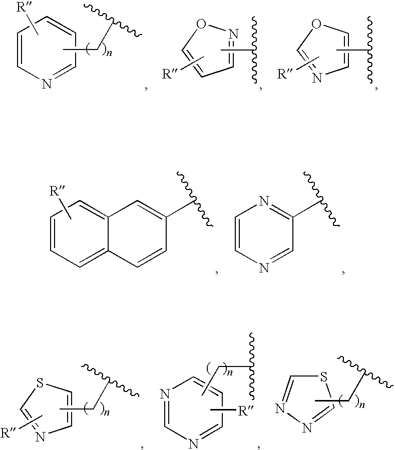

or a substituted lower alkyl; wherein R″ represents one or more substituents each independently selected from aromatic, aliphatic, or alkyl interrupted by heteroatoms and n=0-5.

In one embodiment, the R² of the compound is selected from —Cl and —F. In another embodiment, R², R³ and R⁴ of the compound are independently selected from —Cl, —F and —H. In some embodiments, R⁶ is —CO₂H. In some embodiments, R⁵ is —H. In some embodiments, M is —(CH₂)ₙ— and n=0-5. In some embodiments, M is methylene.

In some embodiments, the compound is represented by any one of Structural Formulas (Ia), (Ib), (Ic), or (Id):

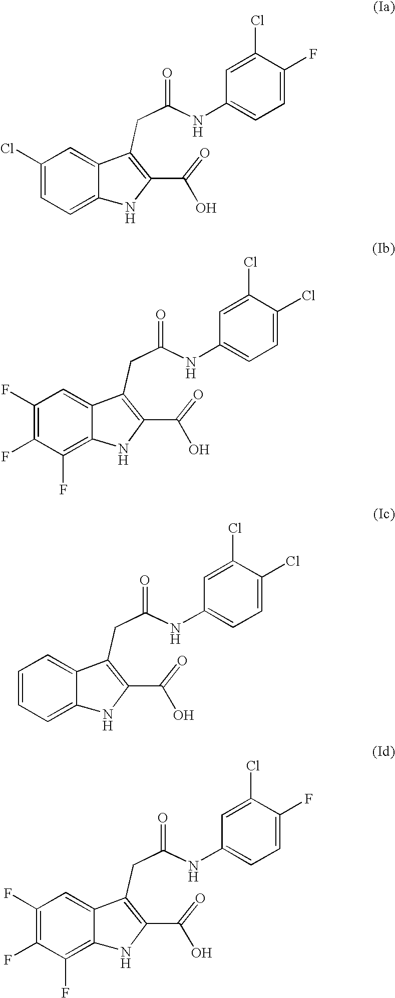

Another aspect of the invention features compounds, as well as pharmaceutical compositions that include the compounds, represented by Structural Formula (II):

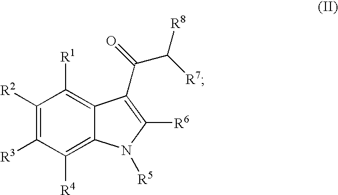

wherein: R¹, R², R³, and R⁴ are each independently selected from —H, —Cl, —F, —CH₃, —Br, —CF₃, —OCF₃, —CN, —CO₂R*, —OR*, —NR*R*, —SO₂R*, and —SO₂NR*R*; R* in each occurrence is independently selected from H, and substituted or unsubstituted alkyl, aryl and alkenyl; R⁵ is —H, lower alkyl, or alkenyl. R⁶ is —CO₂H, —CO₂R′ or —SO₃H; R′ is lower alkyl; R⁷ is aromatic, aliphatic, or alkyl interrupted by one or more heteroatoms; and R⁸ is —H or —CH₃.

In one embodiment, the compound is represented by Structural Formula (IIa):

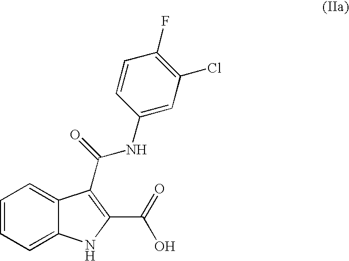

Another aspect of the invention features compounds, as well as pharmaceutical compositions that include the compounds, represented by Structural Formula (III):

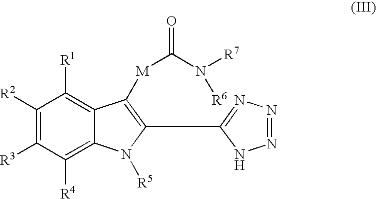

wherein: R¹, R², R³, and R⁴ are each independently selected from —H, —Cl, —F, —Br, —CF₃, —OCF₃, —CN, —CO₂R*, —OR*, —NR*R*, —SO₂R*, and —SO₂NR*R*; R* in each occurrence is independently selected from H, and substituted or unsubstituted alkyl, aryl and alkenyl; R⁵ is —H, lower alkyl, or lower alkenyl; R⁶ is aromatic, aliphatic, or alkyl interrupted by one or more heteroatoms; R⁷ is —H or —CH₃; M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**—, and —(CR**R**)ₙ— and R** independently for each occurrence, is H or lower alkyl; n is 0-5; and n′ is 0-2.

In one embodiment, the compound is represented by Structural Formula (IIIa):

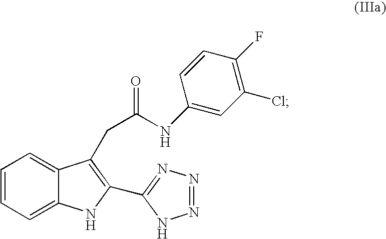

Certain compounds of the present invention can exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms can be present in a substituent such as an alkyl group. All such isomers, as well as mixtures thereof, are intended to be included in this invention.

If, for instance, a particular enantiomer of a compound of the present invention is desired, it can be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers. Moreover, the enantiomers of a racemic mixture can be separated using chiral chromatography, e.g., chiral HPLC.

Contemplated equivalents of the compounds described herein include compounds that otherwise correspond thereto, and which have the same general properties thereof, wherein one or more simple variations of substituents are made which do not adversely affect the efficacy of the compound. In general, the compounds of the present invention can be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

### III. Peptide Conjugates and Intermediates

On aspect of the invention features compounds that enhance peptide exchange conjugated to other molecules, including to macromolecules, nucleic acids, polypeptides, peptides, antibodies, polymers and other small molecules. In one embodiment, the compounds are conjugated to molecules that can bind to MHC Class II molecules, such as those that compete with binding to DR/peptide complexes. Such conjugated molecules can be able to more efficiently displace already bound peptides.

In one embodiment, the compound is represented by Structural Formula (IV):

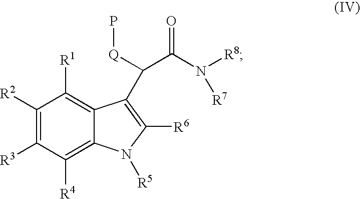

or pharmaceutically acceptable salts of the same, wherein: R¹, R², R³, and R⁴ are each independently selected from —H, —Cl, —F, —CH₃, or —OCH₃; R⁵ is —H, —CH₃, lower alkyl or —(CH₂)₅CH═CH₂; R⁶ is —CO₂H, —CO₂R′; —SO₃H, aliphatic, or aromatic; R′ is lower alkyl; R⁷ is aromatic, aliphatic, or alkyl interrupted by one or more heteroatoms; R⁸ is —H or —CH₃; Q is a covalent bond or an inert linking group or a substituted inert linking group; and P is a polypeptide, peptide, antigen, peptidomimetic, nucleic acid, polymer or other macromolecule. In some embodiments, P is a peptide that loads onto MCH Class II molecules. Further embodiments for P are provided below.

In some embodiments, Q is M and M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**—, and —(CR**R**)ₙ— and R** independently for each occurrence, is H or lower alkyl.

In some embodiments, Q is a C₁₋₁₅ alkyl group wherein one or two methylene groups are optionally replaced by a group Y (provided that none of the Y groups are adjacent to each other), wherein each Y, independently for each occurrence, is selected from —O—, C(═X), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**—.

In some embodiments, Q is a C₁₋₁₅ alkyl group wherein one or two methylene groups are optionally replaced by a group Y (provided that none of the Y groups are adjacent to each other), wherein each Y, independently for each occurrence, is selected from —NR**—, —NR**CO—, and —C(O)NR**—.

In some embodiments, Q is a C₁₋₁₅ alkyl group wherein the methylene group adjacent to said P in said C₁₋₁₅ alkyl group is replaced by Y selected from —NR**—, —NR**CO—, and —C(O)NR**—.

In some embodiments, Q is —(C₃₋₈ alkyl)-NHC(O)—(C₁₋₄ alkyl)-NH—.

In some embodiments, Q is —(C₆ alkyl)-NHC(O)—(C₂ alkyl)-NH—.

In some embodiments, Q is —(C₃₋₈ alkyl)-NH—.

In some embodiments, Q of the structural formula is —(CH₂)ₙ— and n=0-5.

In some embodiments, n is 1.

In another embodiment, the structure of the compound conjugated to a molecule is represented by Structural Formula (I):

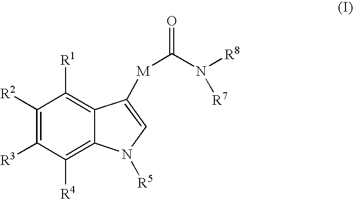

wherein: R¹, R², R³, and R⁴ are each independently selected from —H, —Cl, —F, —CH₃, —Br, —CF₃, —OCF₃, —CN, —CO₂R*, —OR*, —NR*R*, —SO₂R*, and —SO₂NR*R*; R* in each occurrence is independently selected from H, and substituted or unsubstituted alkyl, aryl and alkenyl; R⁵ is —H, -lower alkyl, or lower alkenyl; R⁶ is —CO₂H, —CO₂R′, —SO₃H or SO₃R′; R′ is lower alkyl; R⁷ is aromatic, aliphatic; or alkyl interrupted by one or more heteroatoms; R⁸ is —H or —CH₃ and M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**—, —(CR**R**)ₙ—, and —CR**(-M-P)— and R** independently for each occurrence, is H or lower alkyl; P is a P is a polypeptide, peptide, antigen, peptidomimetic, nucleic acid, polymer or other macromolecule; n is 0-5; and n′ is 0-2.

In one embodiment, P is a polypeptide having at least 50, 75, 100, 150, 200, 300, 400, 500, 1000, or 2000 amino acid residues. In another embodiment, P is a peptide having about 2-50, 2-40, 5-40, 5-35, 10-35, 10-30, 15-30 or about 15-25 amino acid residues. In some embodiments, the compound is conjugated at the N-terminus of the protein or polypeptide. In some embodiments, the compound is conjugated at the C-terminus of the protein or polypeptide. In some embodiments, the compound is conjugated at both the N-terminus and the C-terminus of the protein or polypeptide. In some embodiments, the compound is conjugated to an internal amino acid residue, such as to a lysine or cysteine residue. In one embodiment, the ratio of peptide/polypeptide to compound is about 1:1. In some embodiments, it is about 1:2, 1:3, 1:4, 1:5, 1:10 or greater.

In one embodiment, the peptide or polypeptide includes one or more unnatural amino acids. In one embodiment, the unnatural amino acid is selected from O-methyl-L-tyrosine, L-3-(2-naphthyl)-alanine, 3-methyl-L-phenylalanine, fluorinated phenylalanine, p-benzoyl-L-phenylalanine, p-iodo-L-phenylalanine, p-bromo-L-phenylalanine, p-amino-L-phenylalanine, 3,4-dihydroxy-L-phenylalanine, and isopropyl-L-phenylalanine.

In other embodiments, the unnatural amino acid is selected from azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, and pipecolic acid.

In some embodiments, the peptide or polypeptide includes one or more amino acid analogs. An “amino acid analog” is structurally similar to a naturally occurring amino acid molecule as is typically found in native polypeptides, but differs in composition such that either the C-terminal carboxy group, the N-terminal amino group, or the side-chain functional group has been chemically modified to another functional group. Amino acid analogs include natural and unnatural amino acids which are chemically blocked, reversibly or irreversibly, or modified on their N-terminal amino group or their side-chain groups, and include, for example, methionine sulfoxide, methionine sulfone, S-(carboxymethyl)-cysteine, S-(carboxymethyl)-cysteine sulfoxide and S-(carboxymethyl)-cysteine sulfone. Amino acid analogs can be naturally occurring, or can be synthetically prepared. Non-limiting examples of amino acid analogs include aspartic acid-(beta-methyl ester), an analog of aspartic acid; N-ethylglycine, an analog of glycine; and alanine carboxamide, an analog of alanine. Other examples of amino acids and amino acids analogs are listed in Gross and Meienhofer, The Peptides: Analysis. Synthesis. Biology, Academic Press, Inc., New York (1983).

In some embodiments, P is a pan DR peptide. Pan DR peptides are described in U.S. Pat. No. 5,736,142, Pan DR peptides are peptides of between about 4 and about 20 residues that bind antigen binding sites on MHC molecules encoded by substantially all alleles of a DR locus. These peptides can be used to inhibit immune responses associated with immunopathologies, such as autoimmunity, allograft rejection and allergic responses.

In some embodiments, the peptides are those MHC-class II binding peptides described in U.S. Pat. No. 6,800,730.

In other embodiments, P is a tolerogenic peptide. Administration of tolerogenic peptides antigens has been demonstrated as an effective means of inhibiting disease in experimental autoimmune encephalomyelitis (EAE—a model for multiple sclerosis (MS)) (Metzler and Wraith (1993) Int. Immunol. 5:1159-1165; Liu and Wraith (1995) Int. Immunol. 7:1255-1263; Anderton and Wraith (1998) Eur. J. Immunol. 28:1251-1261); and experimental models of arthritis, diabetes, and uveoretinitis (reviewed in Anderton and Wraith (1998) as above). This has also been demonstrated as a means of treating an ongoing disease in EAE (Anderton and Wraith (1998) as above). U.S. Pat. No. 7,071,297 described exemplary tolerogenic peptides derived from myelin basic protein (MBP).

In some embodiments, P is a branched polypeptide (see, e.g., Hudecz et al., 1988, Biophys. Chem., 31:53-61; Mezo et al., 1989, Biopolymers, 28:1801-26; Hilbert et al., 1994, Scand. J. Immunol., 40:609-617; Toth et al., 1993, Pept. Res., 6:272-280.

In some embodiments, P is a copolymer (e.g., glatiramer acetate). In one embodiment, the peptide is copolymer 1 (Cop-1). Copolymer-1 is a mixture of polypeptides composed of alanine, glutamic acid, lysine, and tyrosine in a molar ratio of approximately 6:2:5:1, respectively. It is synthesized by chemically polymerizing the four amino acids forming products with average molecular weights of 23,000 daltons (U.S. Pat. No. 3,849,550). Cop-1 binds promiscuously, with high affinity and in a peptide-specific manner to purified MS-associated HLA-DR2 (DRB1*1501) and rheumatoid arthritis-associated HLA-DR1 (DRB1*0101) or HLA-DR4 (DRB1*0401) molecules (Fridkis-Hareli et al. (1999) J. Immunol., 162:4697-4704). Cop-1 has been approved as a treatment for relapsing multiple sclerosis (MS). Evidence demonstrates that Cop-1 induces active suppression of CNS-inflammatory disease in animal models (Aharoni et al. (1997) P.N.A.S., 94:10821-26). In humans, glatiramer acetate treatment was found to lead to a significant reduction in the mean annual relapse rate and stabilization of disability. The treatment was accompanied by an elevation of serum IL-10 levels, suppression of the pro-inflammatory cytokine TNF alpha mRNA, and an elevation of the anti-inflammatory cytokines TGF-beta and IL4 mRNAs in PBLs (Miller et al. (1998) J. Neuroimmunol., 92:113-121).

In other embodiments, the peptide is a therapeutic ordered peptide as described in U.S. Pat. No. 7,070,780.

In one embodiment, the peptide is a fragment of pathogen-derived hepatitis B surface and core antigen helper T cell epitopes, pertussis toxin helper T cell epitopes, tetanus toxin helper T cell epitopes, measles virus F protein helper T cell epitopes, Chlamydia trachomatis major outer membrane protein helper T cell epitopes, diphtheria toxin helper T cell epitopes, Plasmodium falciparum circumsporozoite helper T cell epitopes, Schistosoma mansoni triose phosphate isomerase helper T cell epitopes, and Escherichia coli TraT helper T cell epitopes. These fragments can have a length of about 2-50, 2-40, 5-40, 5-35, 10-35, 10-30, 15-30 or about 15-25 amino acid residues.

The peptides can be produced in a solid-phase-synthetic manner in polymer resins. Details are known to one skilled in the art. Literature: Peptide chemistry—A Practical Textbook (M. Bodanszky), 2nd Edition, Springer-Verlag Heidelberg 1993; Anti-Cancer Drug Design 12, 145 167, 1997; J. Am. Chem. Soc. 117, 118212, 1995. U.S. Pat. Pub. No. 2007/0004905 describes a method of solid-phase peptide synthesis.

In one embodiment, the peptides can be made by chemical synthesis methods which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

After complete assembly of the desired peptide immunogen, the resin is treated according to standard procedures to cleave the peptide from the resin and deblock the functional groups on the amino acid side chains. The free peptide is purified, for example by HPLC, and characterized biochemically, for example, by amino acid analysis, mass spectrometry, and/or by sequencing. Purification and characterization methods for peptides are well known to those of ordinary skill in the art.

Longer synthetic peptides can be synthesized by well-known recombinant DNA techniques. Many standard manuals on molecular cloning technology provide detailed protocols to produce the peptides described herein by expression of recombinant DNA and RNA. To construct a gene encoding a peptide having a specific sequence, the amino acid sequence is reverse translated into a nucleic acid sequence, preferably using optimized codon usage for the organism in which the gene will be expressed. Next, a gene encoding the peptide is made, typically by synthesizing overlapping oligonucleotides which encode the peptide and necessary regulatory elements. The synthetic gene is assembled and inserted into the desired expression vector. The synthetic nucleic acid sequences encompassed by this invention include those which encode the peptides described herein, immunologically functional homologs, and nucleic acid constructs characterized by changes in the non-coding sequences that do not alter the immunogenic properties of the peptide encoded thereby. Nucleic acids which include sequences that encode the peptides of this invention are also provided. The synthetic gene is inserted into a suitable cloning vector and recombinants are obtained and characterized. The peptide is then expressed under conditions appropriate for the selected expression system and host. The peptide is purified and characterized by standard methods.

The active compounds can be produced separately and then, as part of the solid-phase-synthetic production of the peptides, the active compounds are coupled to the peptides, and the conjugated peptides are then obtained as highly pure compounds after cleavage from resin and purification. Active compounds with linkers that contain carboxyl groups that can be activated with common reagents can be coupled to amino groups of the peptide, such as to the amino group of lysine residues or to the N-terminal peptide-amino group. In addition, linkers with haloalkyl or haloacetyl radicals can be coupled to thiol groups of the peptide, especially the amino acid cysteine or homocysteine.

In some embodiments, a single activatable group is used. The advantage of only one activatable group, such as, e.g., a carboxyl group, or an already activated group, such as, e.g., an isothiocyanate, a haloalkyl group or a haloacetyl group, is that a chemically uniform coupling can be carried out. The haloacetyl group has the special advantage that a chemically uniform coupling to the mercapto group of the cysteine or homocysteine can be carried out. This coupling can be carried out in solution to the unbonded peptide from which protective groups have been removed. By the activated groups, a coupling to peptides is possible without secondary reactions occurring. Methods of conjugating small molecules to peptides are also described in U.S. Pat. No. 6,217,845.

The present invention also contemplates compounds that are useful in preparing peptide conjugates, such as the compounds represented by Formulas (Va), (Vb), and (Vc):

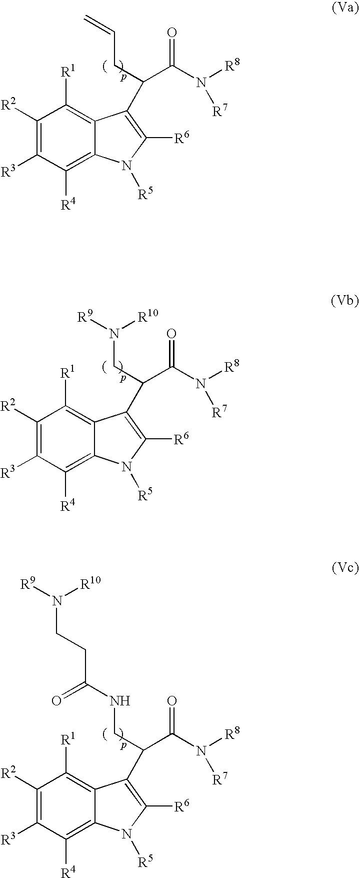

or salts thereof, wherein:
R¹, R², R³, and R⁴ are each independently selected from —H, —Cl, —F, —CH₃, —Br, —CF₃, —OCF₃, —CN, —CO₂R*, —OR*, —NR*R*, —SO₂R*, and —SO₂NR*R*;
R* in each occurrence is independently selected from H, substituted or unsubstituted alkyl, aryl and alkenyl;
R⁵ is —H, -lower alkyl, lower alkenyl, or an amino protecting group;
R⁶ is —CO₂H, —CO₂R′, —SO₃H, —SO₃R′, or tetrazolyl;
R′ is lower alkyl;
R⁷ is aromatic, aliphatic, or alkyl interrupted by one or more heteroatoms;
R⁸ is —H or —CH₃;
R⁹ and R¹⁰ are independently selected from H, alkyl, or an amino protecting group,
or R⁹ and R¹⁰ together form an amino protecting group; and
p is 1 to 15.

In some embodiments, R⁷ of the compound is a substituted phenyl group. In some embodiments, R⁷ is phenyl substituted by one or more halogens. In some embodiments, R⁷ of the compound is a 3,4-dihalo substituted phenyl group where the halogens are independently selected from —Br, —Cl and —F. In some embodiments, R² of the compound is selected from —Cl and —F. In some embodiments, R², R³ and R⁴ of the compound are independently selected from —Cl, —F and —H. In some embodiments, R⁶ is —CO₂H or —CO₂R′. In some embodiments, R⁵ is —H or an amino protecting group. In some embodiments, p is 2 to 10, 5 to 7, or 6. In some embodiments, R⁹ and R¹⁰ are independently selected from H or an amino protecting group. In some embodiments, R⁹ and R¹⁰ together form an amino protecting group.

### IV. Methods of Treating Autoimmune Disorders

A number of different treatment approaches for autoimmune diseases require binding of the therapeutic compound to MHC-II. These compounds fall into three categories: (1) Peptides and altered peptide ligands of self-antigens that induce T cell tolerance when administered under non-inflammatory conditions; (2) Inhibitors that reduce binding of self-peptides by occupying the MHC-II peptide binding groove; (3) Copolymers such as glatiramer acetate that bind to MHC-II and induce the expansion of regulatory CD4 T cells.

Certain MHC-II based therapeutics are already in clinical use, such as glatiramer acetate, a FDA approved drug for the treatment of relapsing-remitting MS. However, they need to be administered in large doses (in the case of glatiramer acetate, daily subcutaneous injection of 20 mg) due to inefficient loading onto MHC-II (Johnson et al., 1998, Neurology, 50:701-708). Loading is limited by proteolytic degradation and peptide competition in the late endosomal compartment in which DM-catalyzed peptide exchange takes place (Trombetta et al., 2003, Science, 299:1400-03).

One aspect of the invention features methods of treating a subject afflicted with an autoimmune disorder that include administering to the subject a therapeutically effective amount of a compound that increases peptide exchange on MHC-class II molecules. In one embodiment, a peptide or an altered peptide ligand that induces self-tolerance is also administered to the subject, optionally conjugated to the compound, such as to the C-terminus. In another embodiment, an inhibitor that reduces binding of self-peptides by occupying the MHC-II peptide binding groove are also administered to the subject. In another embodiment, copolymers are also administered to the subject.

One aspect of the invention features a methods of treating a subject afflicted with an autoimmune disorder that include administering to the subject a therapeutically effective amount of a compound that increases peptide exchange on MHC-class II molecules and which is conjugated to any one of (i) a peptide or an altered peptide ligand that induces self-tolerance is also administered to the subject, (ii) an inhibitors that reduces binding of self-peptides by occupying the MHC-II peptide binding groove, or (iii) a copolymer.

In one embodiment, the compound that is administered to the subject is represented by Structural Formula (I), (Ia), (Ib), (Ic), (Id), (II), (IIa), (III), (IIIa), (IV), (Va), (Vb), or (Vc) as defined herein. In one embodiment, the compound is one of the compounds listed in Tables 1-6. In one embodiment, the compound is one of the compounds listed in Tables 1-6 having an activity level of “+”, “++”, “+++”, “++++”, “+++++” or “++++++”. In one embodiment, the compound used is anyone of compounds A1-A87, or more preferably anyone of compounds A1-A87 also having at least a “+” level of activity. In one embodiment, the compound is represented by structural formula (IV) as defined herein, wherein “P” is (i) a peptide or an altered peptide ligand that induces self-tolerance is also administered to the subject, (ii) an inhibitors that reduces binding of self-peptides by occupying the MHC-II peptide binding groove, or (iii) a copolymer. In one embodiment, the constituents on formula (IV) correspond to those compounds A1-A87 or (Ia), (Ib), (Ic), or (Id).

In one embodiment, the autoimmune disease is selected from multiple sclerosis, type-I diabetes (IDDM), Hashinoto's thyroiditis, Crohn's disease, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barré syndrome, psoriasis and myasthenia gravis.

In one embodiment, the compound is coadministered with, or conjugated to, glatiramer acetate and preferably used to treat multiple sclerosis. A phase III clinical trial demonstrated that glatiramer acetate reduces the frequency of relapses in relapsing-remitting MS. In this trial, glatiramer acetate was injected subcutaneously at a daily dose of 20 mg over a course of two years and found to reduce the relapse rate by 29% (Johnson et al., 1998, Neurology, 50:701-708). The therapeutic effect of glatiramer acetate is thus in a similar range to that of β-interferon, and these two compounds are now the mainstay of therapy for MS (Johnson, 1997, J. Neural Transm. Suppl. 49:111-115; Johnson et al., 1998, Neurology, 50:701-708).

Glatiramer acetate is an unusual compound, because it represents a random polymer composed of four amino acids, L-tyrosine, L-glutamic acid, L-alanine and Lysine in a specific molar ratio of 1.0, 1.4, 4.2 and 3.4, respectively (Sela and Teitelbaum, 2001, Expert Opin. Pharmacother., 2:1149-65). Studies have demonstrated that glatiramer acetate binds to multiple DR molecules, including DR2, and that glatiramer acetate occupies the peptide binding site (Fridkis-Hareli and Strominger, 1998, J. Immunol., 160:4386-97). The biological properties of this copolymer are thus due to binding to the MHC-II groove. The compound that is currently in clinical use is generated by a polymerization reaction which generates molecules with a range of different molecular weights, but more recent studies have shown that a 50-mer synthesized by solid phase peptide methodology with the same amino acid composition has properties similar to glatiramer acetate in animal models (Fridkis-Hareli et al., 2002, J. Clin. Invest., 109:1635-43).

The therapeutic efficacy of glatiramer acetate in MS can be limited by inefficient loading onto the MHC-II anti proteolysis. Proteolysis can be a significant issue for a random copolymer like glatiramer acetate, because it lacks a defined three-dimensional structure. The compounds disclosed herein can accelerate loading of glatiramer acetate, especially in the early endosomal compartment with low protease activity or at the cell surface, and substantially increase presentation of glatiramer acetate derived peptides by DR molecules.

The compounds of this invention are useful for enhancing the efficacy of MHC class II based therapeutics for autoimmune diseases, such as MHC class II blockers, peptides used for tolerance induction or glatiramer acetate.

In another embodiment, the compound is coadministered with, or conjugated to a pan DR peptide. In another embodiment, the compound is coadministered with, or conjugated to MHC class II binding peptides described in U.S. Pat. No. 6,800,730. In another embodiment, the compound is coadministered with, or conjugated to a tolerogenic peptide. In another embodiment, the compound is coadministered with, or conjugated to copolymer. In another embodiment, the compound is coadministered with, or conjugated to a therapeutic ordered peptide, such as those described in U.S. Pat. No. 7,070,780.

### V. Polypeptide Display on Antigen Presenting Cells

The compounds described herein promote the binding of peptides to DR molecules and substantially reduces the dose of peptide required for an equivalent level of presentation (˜10-fold). As such, DR molecules can be used as a display platform for immunomodulatory molecules. Since high-affinity peptides have long half-lives on DR molecules on the cell surface, DR-hound peptides can be used as anchors for long-lived display of polypeptides of interest (e.g., cytokines) on the cell surface (see FIG. 1B). This polypeptide display system can be useful, e.g., for treatment of inflammatory diseases. Without wishing to be bound by theory, applicants believe that T cells will migrate through secondary lymphoid structures and form stable interactions. These interactions will last for many hours during which the APC presents a peptide/MHC complex, which is then recognized by the TCR where the display of the polypeptides via MHC class II molecules concentrates polypeptides at the site where T cell differentiation occurs. The polypeptides (e.g., cytokines) present at that site determine differentiation of T cells into subsets with either an effector or regulatory phenotype. One use of these methods is to improve the efficacy of cytokines that down-modulate chronic inflammatory responses. This approach can modulate immune responses in a variety of diseases, including autoimmune diseases, allergic diseases and organ transplantation. Polypeptide (e.g., cytokine) display can also be used to enhance T cell responses to induce differentiation of long-lived memory T cells with effector properties (i.e., IL-15).

Exemplary polypeptides that can be displayed include, but are not limited to, the cytokines IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-β, IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

### VI. Methods of Boosting Immunity

The compounds described herein can be used to modulate immune responses. For example, the compounds described herein can be used to alter the kinetics of peptide exchange, thereby affecting a subject's repertoire of immune cells specific for an antigen. By influencing peptide exchange, the compounds described herein can provide an increase in cells and antibodies with a higher affinity for an antigen. The compounds of the present invention also can be useful in treating a subject with a condition where an increased CD4 T cell response would benefit the subject. The compounds of the present invention also can be useful in treating a subject with a condition where an increase in HLA-DM activity would benefit the subject. The compounds described herein also can be useful for treating viral infections, enhancing tumor immunity, enhancing vaccination efficacy or in ameliorating immune suppression. The compounds described herein can be useful for enhancing the efficacy of vaccines, such as to treat infectious agents and/or cancer.

One aspect described herein features methods for the treatment of subjects having or at risk of having a disease and/or in a state of immunosuppression. For example, the subjects can have or be at risk of developing an infectious disease. In another example, the subject can have or be at risk of developing a cancer. In another example, the subjects can have or can be at risk of developing an immune system suppression, such as from a genetic condition, radiation treatment, chemotherapy, or an infection, such as a chronic infection. Subjects with abnormally low CD4 cell counts are one example of immune suppressed subjects. In general, the number of functional CD4⁺-T cells that is within a normal range is known for various mammalian species. In human blood, e.g., the number of functional CD4⁺-T cells which is considered to be in a normal range is from about 600 to about 1500 CD4⁺-T cells/mm³ blood. An individual having a number of CD4⁺-T cells below the normal range, e.g., below about 600/mm³, can be considered “CD4⁺-deficient.”

Subjects can be exposed to myeloid, lymphoid or general immune suppressing conditions by the use of either immunosuppressant drugs such as cyclosporin or high dose chemotherapeutic compounds which affect dividing hematopoietic cells. Immunosuppression can also arise as a result of treatment modalities such as total body irradiation or conditioning regimens prior to bone marrow transplantation. Viral infection, particularly as in the case of infection with human immunodeficiency virus (HIV), can also immunosuppress an individual. In some embodiments, subjects are those which have not been exposed and are not anticipated to be exposed to the above-mentioned conditions. In other embodiments, the instant invention aims to treat subjects who can have been myelosuppressed or immunosuppressed (e.g., by exposure to one or more of the above conditions).

The invention thus involves treatment in some embodiments of individuals who are immunocompromised and in other embodiments who are not immunocompromised. Subjects who are not immunocompromised are those that have blood cell counts in the normal range. Subjects who are immunocompromised are those that have blood cell counts below the normal range. Normal ranges of blood counts are known to the medical practitioner and reference can be made to a standard hematology textbook for such counts. In addition, reference can be made to published PCT application PCT/US00/14505.

As mentioned above, the subject can have or be at risk of developing an infectious disease. The agents described herein thus can be used to inhibit or treat infectious diseases such as bacterial, viral, fungal, parasitic and myobacterial infections. The compounds described herein that increase peptide exchange, whether conjugated to other molecules or unconjugated, can also be used prophylactically to inhibit or reduce the incidence of infection during periods of heightened risk, including for example flu season, epidemics, and travel to places where the risk of pathogen exposure is high. The compounds described herein can prepare a subject for passive exposure to a pathogen.

Subjects having an infectious disease are those that exhibit symptoms of infectious disease (e.g., rapid onset, fever, chills, myalgia, photophobia, pharyngitis, acute lymphadenopathy, splenomegaly, gastrointestinal upset, leukocytosis or leukopenia) and in whom infectious pathogens or byproducts thereof can be detected. Tests for diagnosing infectious diseases are known in the art and the ordinary medical practitioner will be familiar with these laboratory tests which include but are not limited to microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject at risk of developing an infectious disease is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

The compounds described herein also are used to treat subjects having or at risk of developing cancer. A subject having a cancer is a subject that has detectable cancerous cells. A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

The compositions and methods described herein in certain instances can be useful for replacing existing surgical procedures or drug therapies, although in most instances the present invention is useful in improving the efficacy of existing therapies for treating such conditions. Accordingly combination therapy can be used to treat the subjects that are undergoing or that will undergo a treatment for, inter alia, infectious disease or cancer. For example, the compounds of the present invention can be administered in conjunction with anti-microbial agents or anti-proliferative agents. The compounds described herein also can be administered in conjunction with other immunotherapies, such as with antigens, adjuvants, immunomodulators, or passive immune therapy with antibodies. The compounds described herein also can be administered in conjunction with nondrug treatments, such as surgery, radiation therapy or chemotherapy. The other therapy can be administered before, concurrent with, or after treatment with the compounds described herein. There can also be a delay of several hours, days and in some instances weeks between the administration of the different treatments, such that the compounds described herein can be administered before or after the other treatment.

The compounds described herein also are used with nondrug treatments for cancer, such as with surgical procedures to remove the cancer mass, chemotherapy or radiation therapy. The nondrug therapy can be administered before, concurrent with, or after treatment with the compounds described herein. There can also be a delay of several hours, days and in some instances weeks between the administration of the different treatments, such that the compounds described herein can be administered before or after the other treatment.

The invention in one embodiment contemplates the use of compounds described herein in cancer subjects prior to surgery, radiation or chemotherapy in order to create memory immune cells to the cancer antigen. In this way, memory cells of the immune system can be primed with cancer antigens and thereby provide immune surveillance in the long term. Immune cells so primed can invade a tumor site and effectively clear any remaining tumor debris following the other treatment.

The invention also contemplates the use of compounds described herein together with other immunotherapies. In one embodiment, the other immunotherapy is treatment with an antigen such as a cancer antigen or a microbial antigen (bacterial antigens, viral antigens, fungal antigens and parasitic antigens). The antigens can be whole antigens, antigen fragments such as peptides, genetically modified antigens, antigens contained in lysates, and the like. The vaccine methods and compositions described herein similarly envision the use of nucleic acid based vaccines in addition to peptide based vaccines. The art is familiar with nucleic acid based vaccines. In one embodiment, the compounds are conjugated with the antigen. In one embodiment, the conjugates are represented by structural formula (IV), wherein P is the antigen. In some embodiment, the compounds structural formula (IV), wherein P is the antigen and the R¹ through R⁸ corresponds to those of Structural Formulas (Ia), (Ib), (Ic), (Id) or any one of compounds A1-A87. In one embodiment, the antigen represented by P has at least 50, 75, 100, 150, 200, 300, 400, 500, 1000, or 2000 amino acid residues. In another embodiment, the antigen represented by P has about 2-50, 2-40, 5-40, 5-35, 10-35, 10-30, 15-30 or about 15-25 amino acid residues.

Antigens associated with infectious diseases that can be used in the methods described herein include whole bacteria, whole virus, whole fungi, whole parasites, fragments thereof, lysates thereof, killed versions thereof, etc. The compounds described herein can be used in combination with various vaccines either currently being used or in development, whether intended for human or non-human subjects.

The compound described herein can be used with cancer antigens. A cancer antigen as used herein is a compound differentially associated with a cancer, preferably at the cell surface of a cancer cell (or even at the surface of the neovasculature), that is capable of invoking an immune response. The antigen invokes an immune response when it is presented (in a digested form) on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art. In one embodiment, the compounds are conjugated with the cancer antigen. In one embodiment, the conjugates are represented by structural formula (IV), wherein P is the cancer antigen. In some embodiment, the compounds structural formula (IV), wherein P is the antigen and the R¹ through R⁸ corresponds to those of Structural Formulas (Ia), (Ib), (Ic), (Id) or any one of compounds A1-A69. In one embodiment, the cancer antigen represented by P has at least 50, 75, 100, 150, 200, 300, 400, 500, 1000, or 2000 amino acid residues. In another embodiment, the cancer antigen represented by P has about 2-50, 2-40, 5-40, 5-35, 10-35, 10-30, 15-30 or about 15-25 amino acid residues.

A cancer antigen encompasses antigens that are differentially expressed between cancer and normal cells. Due to this differential expression, these antigens can be targeted in anti-tumor therapies. Cancer antigens can be expressed in a regulated manner in normal cells. For example, they can be expressed only at certain stages of differentiation or at certain points in development of the organism or cell. Some are temporally expressed as embryonic and fetal antigens. Still others are never expressed in normal cells, or their expression in such cells is so low as to be undetectable.

Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

The invention also seeks to enhance other forms of immunotherapy including dendritic cell vaccines. These vaccines generally include dendritic cells loaded ex vivo with antigens such as tumor-associated antigens. The dendritic cells can be incubated with the antigen, thereby allowing for antigen processing and expression on the cell surface, or the cells can simply be combined with the antigen prior to injection in viva. Alternatively, the dendritic cells can be activated in vitro and then re-infused into a subject in the activated state. Compounds described herein, whether conjugated or not, can be combined with the dendritic cells in all of these embodiments. Examples of dendritic cell based vaccines include autologous tumor antigen-pulsed dendritic cells (advanced gynecological malignancies); blood-derived dendritic cells loaded ex vivo with prostate cancer antigen (Provenge; Dendreon Corporation); blood-derived dendritic cells loaded ex vivo with antigen for multiple myeloma and other B-cell malignancies (Mylovenge; Dendreon Corporation); and blood-derived dendritic cells loaded ex vivo with antigen for cancers expressing the HER-2/neu proto-oncogene (APC8024; Dendreon Corporation); xenoantigen (e.g., PAP) loaded dendritic cells, and the like.

The compounds described herein also can be used in conjunction with passive immune therapy. The antibodies that can be used with the conjugated and unconjugated compounds described herein include those useful in cancer and infectious disease as well as other disorders for which antibodies and antigens have been identified and which would benefit from an enhanced immune response.

The antibodies or fragments thereof useful in the invention can be specific for any component of a particular target. Accordingly, the antibody can recognize and bind to proteins, lipids, carbohydrates, DNA, RNA, and any combination of these in molecular or supra-molecular structures (e.g., cell organdies such as mitochondria or ribosomes). The antibody or fragment thereof can also recognize a modification of the tumor cell, such as e.g., chemical modifications, or genetic modifications made by transfection ex vivo or in vivo with DNA or RNA. As used herein, the terms “antibody” and “immunoglobulin” are used interchangeably.

Bispecific antibodies can also be used in the invention. A bispecific antibody is one having one variable region that specifically recognizes a tumor antigen and the other variable region that specifically recognizes an antigenic epitope of a host immune effector cell that has lytic or growth inhibitory activity against the tumor. Bispecific and multispecific antibody complexes can be created by linkage of two or more immunoglobulins of different specificity for tumor antigens and/or effector cell antigens, either at the peptide or nucleic acid level.

Immunoglobulin can be produced in vivo in human or non-human species, or in vitro from immunoglobulin encoding DNA or cDNA isolated from libraries of DNA (e.g., phage display libraries). Immunoglobulin can also be modified genetically or chemically to incorporate human polypeptide sequences into non-human coding sequences (commonly referred to as humanization). Additionally, immunoglobulins can be modified chemically or genetically to incorporate protein, lipid, or carbohydrate moieties. Potential modifications could also include naturally occurring or synthetic molecular entities that are either directly toxic for tumor cells or serve as ligands or receptors for biologically active molecules that could suppress tumor growth. For example, growth factors, cytokines, chemokines and their respective receptors, immunologically active ligands or receptors, hormones or naturally occurring or synthetic toxins all represent biologically active molecules that could interact with suitably modified immunoglobulins and their targets.

The compounds described herein can also be combined with other immunomodulatory agents for enhancing an immune response to an antigen, such as cytokines, chemokines, and growth factors, such as those that stimulate hematopoietic cells. Immune responses can be induced or augmented by cytokines or chemokines (Bueler & Mulligan, 1996, Mol. Med., 2:545-555; Chow et al., 1997, J. Virol., 71:169-178; Geissler et al., 1997, J. Immunol., 158:1231-37; Iwasaki et al., 1997, J. Immunol., 158:4591-4601) or B-7 co-stimulatory molecules (Iwasaki et al., 1997, J. Immunol., 158:4591-4601; Tsuji et al., 1997, Eur. J. Immunol., 27:782-787). The cytokines and/or chemokines can be administered directly or can be administered in the form of a nucleic acid vector that encodes the cytokine, such that the cytokine can be expressed in vivo. In one embodiment, the cytokine or chemokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response.

Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

The term “chemokine” is used as a generic name for peptides or polypeptides that act principally to chemoattract effector cells of both innate and adaptive immunity. Chemokines are thought to coordinate immunological defenses against tumors and infectious agents by concentrating neutrophils, macrophages, eosinophils and T and B lymphocytes at the anatomical site in which the tumor or infectious agent is present. In addition, many chemokines are known to activate the effector cells so that their immune functions (e.g., cytolysis of tumor cells) are enhanced on a per cell basis. Two groups of chemokines are distinguished according to the positions of the first two cysteine residues that are conserved in the amino-terminal portions of the polypeptides. The residues can either be adjacent or separated by one amino acid, thereby defining the CC and CXC cytokines respectively. The activity of each chemokine is restricted to particular effector cells, and this specificity results from a cognate interaction between the chemokine and a specific cell membrane receptor expressed by the effector cells. For example, the CXC chemokines IL-8, Groα/β and ENA 78 act specifically on neutrophils, whereas the CC chemokines RANTES, MIP-1α and MCP-3 act on monocytes and activated T cells. In addition, the CXC chemokine IP-10 appears to have anti-angiogenic activity against tumors as well as being a chemoattractant for activated T cells. MIP-1α also reportedly has effects on hemopoietic precursor.

### VII. Pharmaceutical Compositions

The invention further features compositions, including pharmaceutical compositions, that include the compounds described herein, optionally formulated with, and/or conjugated to, peptides/peptidomimetics.

The pharmaceutical formulations described herein contain the compounds described herein, optionally formulated with and/or conjugated to peptides/peptidomimetics, in a pharmaceutically acceptable carrier suitable for administration and/or delivery in vivo. The pharmaceutical compositions of the present invention can be formulated for oral, sublingual, buccal, intranasal, inhalation, injection (subcutaneous, intravenous, intrathecal, intraperitoneal, etc.) or infusion. When administered, the compounds described herein are administered in pharmaceutically acceptable preparations. Such preparations can routinely contain pharmaceutically acceptable concentrations of salt, buffering agents, preservatives, compatible carriers, and the like. The pharmaceutical preparations described herein also can contain immunomodulatory agents, anti-cancer agents, anti-microbials, and/or antigens. Thus, “cocktails” are contemplated. The pharmaceutical composition can be sterile. It can optionally include any one or combination of a buffering agent, a chelating agent, a preservative or an isotonicity agent.

A kit can include, for example, a container containing a first vial that houses a compound described herein. A second vial can contain an antigen and an adjuvant. A syringe can be provided for mixing the contents of the first and second vial. Instructions for operation can also be provided.

Buffers in general are well known to those of ordinary skill in the art. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

Chelating agents are chemicals which form water soluble coordination compounds with metal ions in order to trap or remove the metal irons from solution, thereby avoiding the degradative effects of the metal ions. Chelating agents include ethylenediaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid and derivatives thereof. Citric acid also is known as citric acid monohydrate. Derivatives of citric acid include anhydrous citric acid and trisodiumcitrate-dihydrate. Still other chelating agents include niacinamide and derivatives thereof and sodium desoxycholate and derivatives thereof. Another well known chelating agent is L-glutamic acid, N,N-diacetic acid and derivatives thereof (also known as GLDA). Derivatives include monosodium L-glutamic acid N,N-diacetic acid.

The pharmaceutical preparations described herein also can include isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol. Optionally, the pharmaceutical preparations described herein can further include a preservative. Suitable preservatives include but are not limited to: chlorobutanol (0.3-0.9% W/V), parabens (0.01-5.0%), thimerosal (0.004-0.2%), benzyl alcohol (0.5-5%), phenol (0.1-1.0%), and the like.

In one embodiment, the pharmaceutical compositions further include an anti-cancer agent. In some embodiments the anti-cancer agent is a cytotoxic agent. In other embodiments the anti-cancer agent is an antibody. In one embodiment, the pharmaceutical composition further includes an anti-pathogenic agent. In some embodiments, the anti-pathogenic agent is an anti-viral agent. In other embodiments, the anti-pathogenic agent is an anti-bacterial agent. In one embodiment, the pharmaceutical composition further contains an antigen. In some embodiments, the antigen is a cancer antigen. In other embodiments, the antigen is a viral antigen, a bacterial antigen, a fungal antigen or a parasitic antigen. The pharmaceutical composition also can further include, separate from or in addition to the antigen, an immunomodulatory agent. In some embodiments, the immunomodulatory agent is any one or more of an adjuvant, a hematopoietic cell stimulator, a cytokine, a growth factor, or an immunostimulatory oligonucleotide.

### VIII. Administration of Compositions

The preferred amount of the compounds described herein is a therapeutically effective amount thereof which is also medically acceptable. Actual dosage levels of the pharmaceutical compositions of the present invention can be varied so as to obtain an amount that is effective to achieve the desired therapeutic response for a particular patient, pharmaceutical composition, and mode of administration, without being toxic to the patient. The selected dosage level and frequency of administration will depend upon a variety of factors including the route of administration, the time of administration, the duration of the treatment, other drugs, compounds and/or materials used in combination with the compounds described herein, the age, sex, weight, condition, general health and prior medical history of the patient being treated, and the like factors well known in the medical arts. A physician having ordinary skill in the art can readily determine and prescribe the therapeutically effective amount of the pharmaceutical composition required.

Effective amounts can be determined, for example, by measuring increases in the immune response, for example, by the presence of higher titers of antibody, the presence of higher affinity antibodies, the presence of a desired population of immune cells such as memory cells to a particular antigen, or the presence of particular antigen specific cytotoxic T cells. Effective amounts also can be measured by a reduction in microbial load in the case of an infection or in the size or progression of a tumor in the case of cancer. An effective amount also can be reflected in a reduction in the symptoms experienced by a particular subject being treated.

Dosage can be adjusted appropriately to achieve desired drug levels, locally or systemically. Generally, daily doses of compounds will be from about 0.001 mg/kg per day to 1000 mg/kg per day. It is expected that doses in the range of about 0.1 to 50 mg/kg per day will be effective. In the event that the response in a subject is insufficient at such doses, even higher doses (or effective higher doses by a different, more localized delivery route) can be employed to the extent that patient tolerance permits.

A variety of administration routes are available. The particular mode selected will depend of course, upon the particular drug selected, the severity of the disease state being treated and the dosage required for therapeutic efficacy. The methods of this invention, generally speaking, can be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion. Oral and intravenous routes are preferred. For administration by injection, conventional carriers well known to those of ordinary skill in the art can be used.

Other delivery systems can include time-release, delayed release, or sustained release delivery systems. Such systems can avoid repeated administrations of the conjugates described herein, increasing convenience to the subject and the physician. Many types of release delivery systems are available and known to those of ordinary skill in the art. They include polymer based systems such as polytactic and polyglycolic acid, polyanhydrides and polycaprolactone; wax coatings, compressed tablets using conventional binders and excipients, and the like. Bioadhesive polymer systems to enhance delivery of a material to the intestinal epithelium are known and described in published PCT application WO 93/21906. Capsules for delivering agents to the intestinal epithelium also are described in published PCT application WO 93/19660.

### EXAMPLES

The invention now being generally described, it will be more readily understood by reference to the following examples, which are included merely for purposes of illustration of certain aspects and embodiments of the present invention, and are not intended to limit the invention, as one skilled in the art would recognize from the teachings herein and the following examples, that other DNA microarrays, cell types, agents, constructs, or data analysis methods, all without limitation, can be employed, without departing from the scope of the invention as claimed.

The contents of any patents, patent applications, patent publications, or scientific articles referenced anywhere in this application are herein incorporated in their entirety.

### Example 1

### Expression of DR/CLIP Complexes for Identification of Small Molecules that Modulate Peptide Binding

The assay system that we used was based on the mechanism by which peptides are loaded onto MHC class II molecules in endosomes/lysosomes. This compartment is characterized by an acidic pH (4.5 to 5.5) and the presence of DM, which accelerates the release of CLIP from MHC class II molecules (Busch et al., 2005, Immunol. Rev., 207:242-260). In order to favor the identification of small molecules that modulate this process in the appropriate cellular compartment, we performed the assay with a DR/CLIP complex and a high affinity peptide at an acidic pH. Because invariant chain is highly sensitive to proteases, we generated DR/CLIP complexes as soluble molecules in CHO cells by attaching the CLIP peptide to the N-terminus of the DRβ chain via a linker with a thrombin cleavage site. Thrombin cleavage converts this inactive precursor into the appropriate substrate for the peptide exchange reaction (Day et al., 2003, J. Clin. Invest., 112:831-842). The CLIP peptide inhibits aggregation of DR molecules and binding of irrelevant peptides during biosynthesis and purification.

High-throughput screening required an abundant source of these DR/CLIP precursors. Applicants therefore generated stable transfectants in CHO cells and grew these cells at a high density in bioreactors. The concentration of DR/CLIP complexes in bioreactor supernatants ranged from 10.5 to 64 mg per 100 ml of supernatant, and was therefore much higher than in the Baculovirus system (typically 0.1-0.2 mg per 100 ml).

### Example 2

### Development of a Real-Time Peptide Binding Assay Based on Fluorescence Polarization

MHC class II molecules reside only transiently in the endosomal/lysosomal peptide loading compartment and the kinetics of DM-catalyzed peptide exchange are therefore critical in the selection of the peptide repertoire in vivo (Busch et al., 2005, Immunol. Rev., 207:242-260). Applicants developed a real-time peptide binding assay designed to represent the environment of the peptide loading compartment and used it to search for small molecules that modulate this process. The MBP (85-99) peptide binds with high affinity to DR2 (Wucherpfennig et al., 1994, J. Exp. Med., 179:279-290) and we labeled it with Alexa™-488 because its fluorescence is stable at the acidic pH required for the assay (fluorescein is quenched at pH 5). Since the P5 lysine residue of the MBP peptide was solvent exposed in the structure of the DR2/MBP peptide complex (Smith et al., 1998, J. Exp. Med., 188:1511-20), a maleimide derivative of Alexa™-488 was used to label a MBP peptide analog in which the P5 lysine was substituted by cysteine.

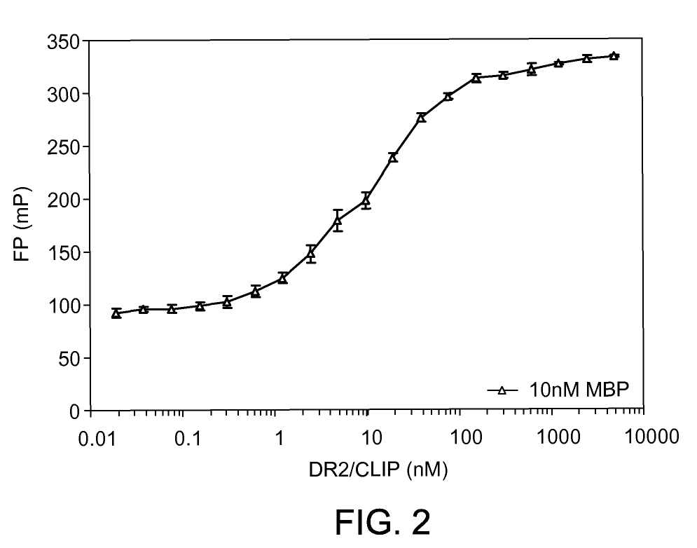

The binding of this fluorescently labeled MBP peptide to DR2 could be followed in real time using a fluorescence polarization (FP) readout as shown in FIG. 2. Polarized fluorescent light was used to excite the fluorophore, and following a 5 ns delay the emitted light was measured both parallel (polarization retained) and perpendicular (polarization lost) to the incident light. Since a small fluorescent peptide tumbles significantly faster than the peptide-receptor complex, the relative intensities of polarized versus scattered emission depend on the ratio of DR-bound versus free fluorescent peptide. Binding of the fluorescent peptide therefore increases the ratio of polarized to non-polarized fluorescent light, which is expressed in milli-polarization units (mP, with maximum polarization corresponding to 1000 mP) (Pin et al., 1999, Anal. Biochem., 275:156-161; Owicki, 2000, J. Biomol. Screen., 5:297-306). FP measurements were made using a IJL Biosystems Analyst HT plate reader (Molecular Devices, Sunnyvale, Calif.) with a 485/20 bandpass, 505 DRLP dichroic, 530/30 bandpass filter set for excitation and emission.

The major advantage of this technique illustrated in FIG. 2 is that the reaction can be read at many time points without the need to withdraw samples for analysis. Most peptide binding assays that are currently used represent end-point assays that are not suitable for the analysis of rapid, early events. Applicants therefore examined whether this assay was suitable to examine the kinetics of small molecule-catalyzed peptide exchange.

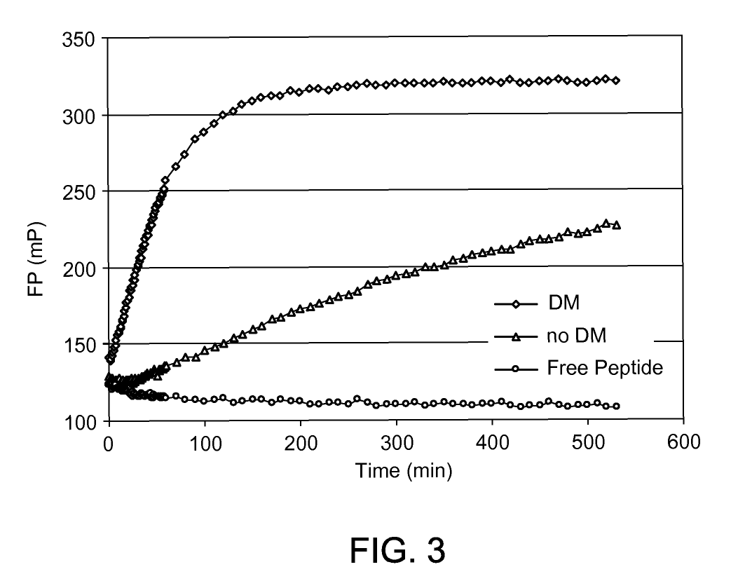

Recombinant DM greatly accelerated the rate of MBP peptide binding, as shown in FIG. 3 by comparison of the reaction kinetics in the absence and presence of 100 nM DM (DR2/CLIP and labeled MBP peptide at 100 nM and 10 nM, respectively). With extended incubation times (18 hours at 37° C.), the same FP values were reached in the presence and absence of DM (data not shown), confirming that DM acts as a catalyst, but does not change the equilibrium of the reaction (Sloan et al., 1995, Nature, 375:802-806; Weber et al., 1996, Science, 274:618-620). These experiments thus demonstrated that fluorescence polarization provides a sensitive, real-time readout of peptide binding to MHC class II molecules ideal for high-throughput screening efforts that require monitoring of the kinetics of the reaction.

### Example 3

### High-Throughput Screening of Large Libraries of Small Molecules

We then used this assay to screen a large and diverse collection of small molecules with the aim of identifying molecules that enhance peptide exchange. The screening was performed using a robotics workstation (Beckman Biomek FX pipette station with a Sagian Core system controlled by SAMI software, Beckman Coulter) in 384-well plates (40 μl volumes, duplicates) with DR2/CLIP at 100 nM, DM at 20 nM and labeled MBP peptide at 10 nM. Fluorescent compounds were excluded based on readings taken before addition of the labeled MBP peptide. The overall fluorescence of each well was also read after peptide addition to ensure that all wells had received equal quantities of labeled MBP peptide. FP values were read at 30, 120 and 360 minutes following initiation of reactions. Reading of each plate required 5 minutes, and plates were therefore set up 5 minutes apart (for details, sec Nicholson et al., 2006, J. Immunol., 176:4208-20).

Using this approach to screen over 100,000 compounds, we identified small molecules that accelerated peptide loading by interacting directly with DR molecules.

### Example 4

### Identification of Compound (Ia), a Small Molecule that Substantially Accelerates Peptide Loading in the Absence of DM

Utilizing the assays described herein Compound (Ia) was identified that dramatically accelerated binding of the labeled MBP peptide to DR2.

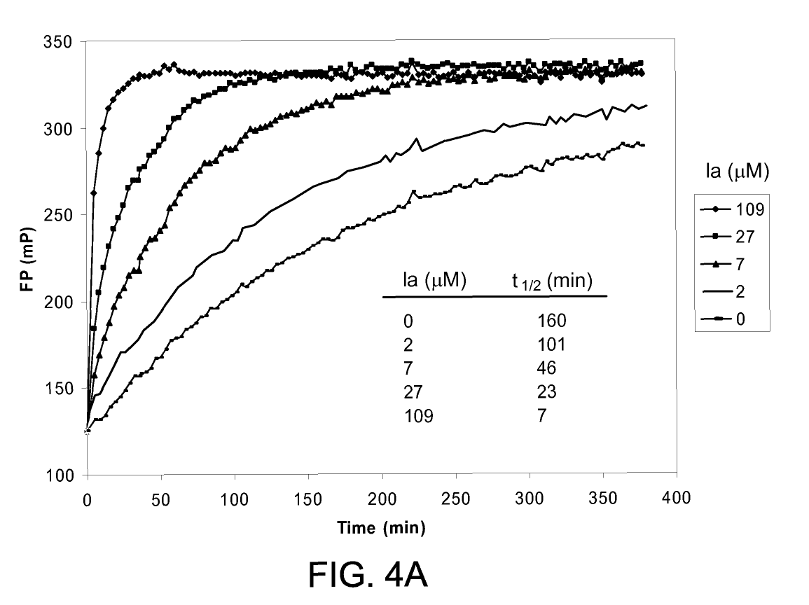

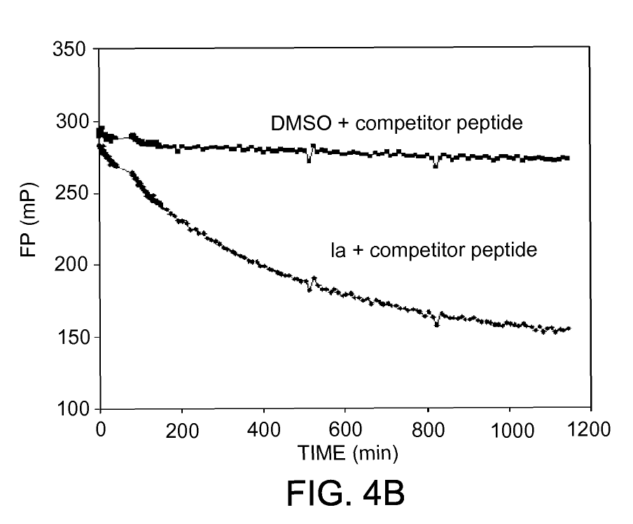

The experiments shown in FIG. 4 here were performed in the absence of DM. In the peptide association experiment (FIG. 4A), DR/CLIP complexes and Alexa™-488 labeled MBP peptide were incubated at pH 5.2 without Compound (Ia) or increasing concentrations of Compound (Ia). The acceleration of peptide binding induced by Compound (Ia) was striking and at the higher Compound (Ia) concentrations the reaction proceeded faster than in the presence of DM. The time to reach half-maximal peptide association was only 7 minutes at the highest Compound (Ia) concentration (109 μM) compared to 160 minutes in the absence of Compound (Ia).

We also examined whether Compound (Ia) could induce the release of a previously bound high affinity peptide from DR2 as shown in FIG. 4B. This issue is important for potential applications of this small molecule because most DR molecules are occupied by intermediate to high affinity peptides. Applicants therefore loaded the DR2 molecule with the high affinity Alexa™-488 MBP peptide during an overnight incubation and then examined if Compound (Ia) could induce dissociation of this labeled peptide. A molar excess of an unlabeled MBP peptide was added to inhibit rebinding of the labeled peptide to DR2. Whereas dissociation of the peptide was very slow in the absence of Compound (Ia) (DMSO control), a marked acceleration of peptide dissociation was observed in the presence of Compound (Ia) (red line). Compound (Ia) thus substantially accelerates peptide binding by creating empty DR molecules.

Certain detergents and phenol alcohols were shown to promote dissociation of low affinity peptides at very high concentrations (milli-molar), but these compounds do not induce dissociation of high affinity peptides (Avva and Cresswell, 1994, Immunity, 1:763-774; Falk et al., 2002, J. Biol. Chem., 277:2709-15). Compound (Ia) induces peptide dissociation at substantially lower concentrations (low micro-molar range) and is capable of triggering release of high affinity peptides from DR2.

### Example 5

### Compound (Ia) Increases the Rate of Peptide Association to Empty DR2 Molecules

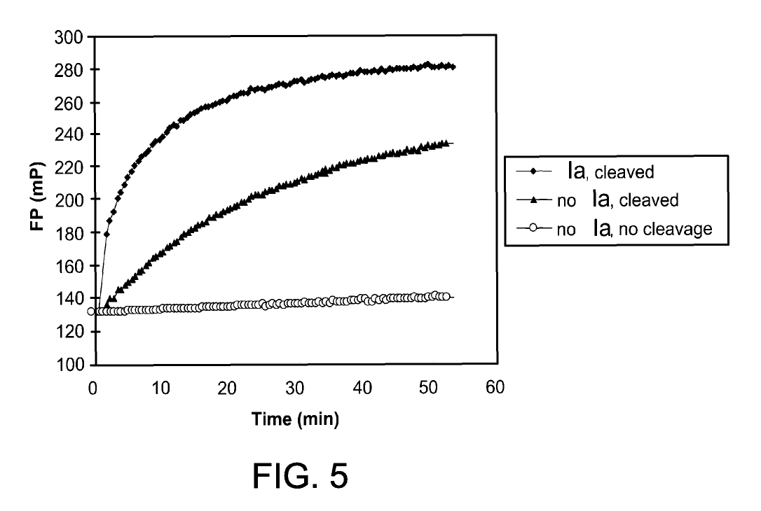

DM acts as a catalyst that accelerates peptide binding reactions and increases both the rate of peptide dissociation and association. Applicants tested whether Compound (Ia) had an activity similar to DM, a hypothesis that implied that it would also accelerate peptide binding to empty DR molecules. Rigorous testing of this hypothesis required generation of empty DR molecules, a task that is challenging because DR molecules aggregate over time in the absence of peptide. Applicants therefore synthesized a peptide with a photo-cleavable residue (a derivative of phenylalanine), so that the binding site could be rapidly vacated by peptide cleavage. This approach has been reported for the creation of empty MHC class I molecules (Toebes et al., 2006, Nat. Med., 12:246-251). The photoreactive group [3-amino-3-(2-nitro) phenyl-propionic acid; DNP] was placed at the P4 position of the peptide, close to the center of DR2 bound peptide. The peptide also carried an N-terminal DNP group for affinity isolation of the complex generated with this peptide. Initial experiments demonstrated that photo-cleavage was efficient and that no full length peptide could be detected by mass spectrometry following UV irradiation for 2 minutes at 360 nm, conditions that do not harm the DR2 protein. Applicants then loaded this peptide onto DR2 molecules, isolated the complex by DNP affinity chromatography, irradiated the samples at 360 nm at 4° C., added Alexa™-488 labeled MBP and tracked its association to DR2 by FP in the presence and absence of Compound (Ia). Applicants observed a striking Compound (Ia)-induced acceleration of Alexa™-488 MBP association to DR2 when the bound peptide was photo-cleaved compared to reactions with photo-cleavage but no Compound (Ia) as diagrammed in FIG. 5. This comparison clearly demonstrated that Compound (Ia) substantially accelerated peptide association to empty DR2 molecules. Like DM, Compound (Ia) can thus accelerate both peptide dissociation (FIG. 4B) and peptide association.

### Example 6

### pH Activity: Compound (Ia) is Active Over a Wide pH Range

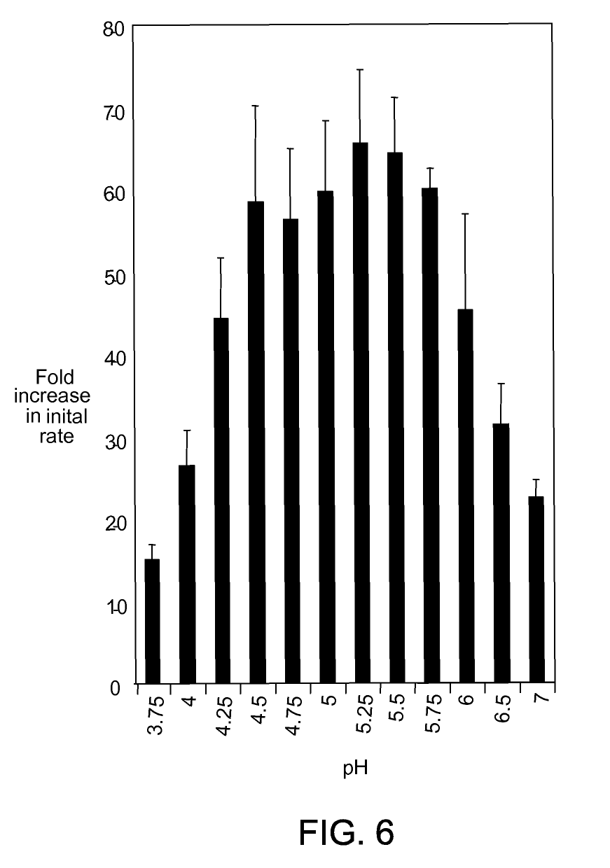

DM is only present in a specialized sub-compartment of the endosomal/lysosomal system and peptides can thus only rapidly bind to DR molecules at this site (Sanderson et al., 1994, Science, 266:1566-69; Schafer et al., 1996, J. Immunol., 157:5487-95). The DM sub-compartment has a low pH (˜5), but earlier endosomal structures have a higher pH (5-6). It was therefore of interest to determine whether Compound (Ia) was also active in this pH range because Compound (Ia)-assisted loading in earlier compartments of the endosomal/lysosomal pathway could protect therapeutics from prolonged exposure to endosomal proteases. Applicants therefore measured the initial rate of the peptide binding reaction in the presence and absence of Compound (Ia) over a wide pH range (3.75-7.0) and calculated the fold increase of the initial rate induced by Compound (Ia) as shown in FIG. 6. The enhancement of peptide association by Compound (Ia) was more than 58-fold over the pH range from pH 4.5 to 5.75. Compound (Ia) even had activity at a neutral pH (22-fold enhancement) and could thus even permit loading of therapeutics to DR molecules on the cell surface.

### Example 7

### Compound (Ia) Increases the Presentation of MBP on MGAR Cells

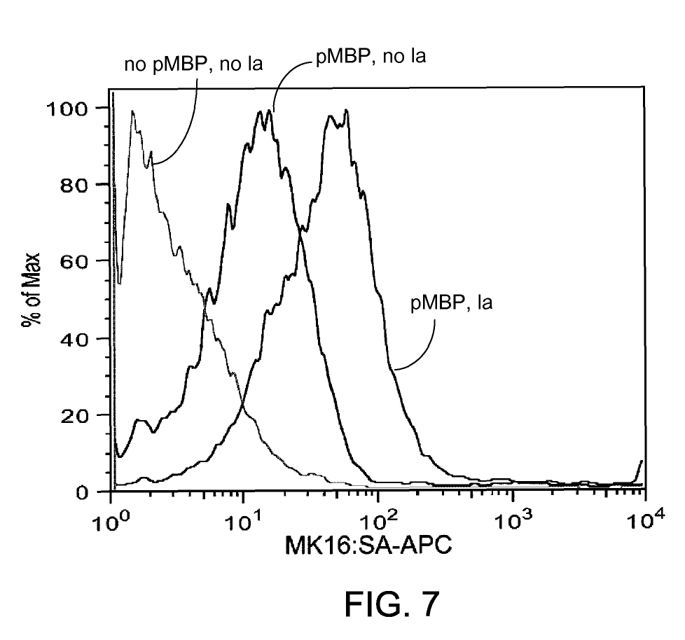

Peptide loading was examined using a human EBV transformed B cell line (MGAR) homozygous for DR2 (DRB1*1501). MBP peptide binding to DR2 was visualized with mAb MK16 that specifically binds to the DR2/MBP peptide complex (Krogsgaard et al., 2000, J. Exp. Med., 191:1395-1412). As diagrammed in FIG. 7, the cells were incubated with Compound (Ia) (100 μM) or without Compound (Ia) (DMSO control) in DMEM media plus 10% FCS and MBP peptide (1.7 μM) for 30 minutes at 37° C. The cells were then labeled with biotinylated MK16 Fab and streptavidin-APC and analyzed by FACS. Cells not pulsed with peptide (green line) were used as a negative control to define background labeling of MK16. In the presence of 100 μM Compound (Ia) the presentation of 1.7 μM MBP on MGAR cells (human EBV transformed B cell line) is significantly increased as measured by staining with a mAb (MK16) that recognizes DR2/pMBP complexes.

Furthermore, the presence of Compound (Ia) lowered the dose of peptide required for equivalent staining approximately 10-fold because the same surface levels of DR2/MBP were observed with 1 μM MBP peptide plus Compound (Ia) compared to 10 μM of peptide and no Compound (Ia).

### Example 8

### Self-Catalyzed Loading Through a Linked Small Molecule with DM-Like Catalytic Function

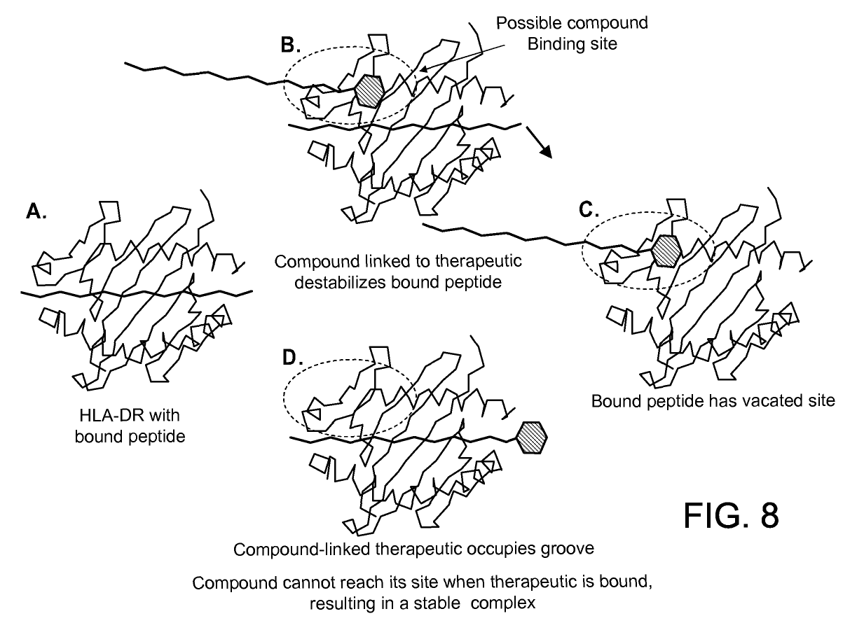

Peptides are bound with long half-lives to DR molecules (Lanzavecchia et al., 1992, Nature, 357:249-252). As shown in FIG. 8A, the peptide exchange catalyst DM is localized to a subset of endosomes and present in sub-stoichiometric quantities relative to DR, normally limiting loading to late endosomal structures (Sanderson et al., 1994, Science, 266:1566-69; Schafer et al., 1996, J. Immunol., 157:5487-95; Busch et al., 2005, Immunol. Rev., 207:242-260). Applicants attached the catalytic Compound (Ia) moiety to MHC-II based therapeutics to improve loading as shown in FIG. 8B. Compound (Ia) mimics the catalytic properties of DM (Sloan et al., 1995, Nature, 375:802-806; Weber et al., 1996, Science, 274:618-620) because it accelerates both peptide dissociation and association. Covalent attachment of Compound (Ia) to a peptide can thus substantially enhance loading of the peptide of interest by creating empty DR molecules in the immediate vicinity as shown in FIG. 8C. Once the Compound (Ia)-linked therapeutic is bound to DR, the Compound (Ia) group may not be able to reach its binding site as shown in FIG. 8D. The Compound (Ia) group would thus create binding sites, but not destabilize the complex once the peptide has bound. A major advantage of covalent attachment is that the Compound (Ia) group cannot diffuse away from the peptide. The physical proximity of Compound (Ia) to the peptide may also strongly favor the Compound (Ia)-linked peptide over other peptides for DR binding.

### Example 9

### Identification of a Linker Attachment Site on Compound (Ia)

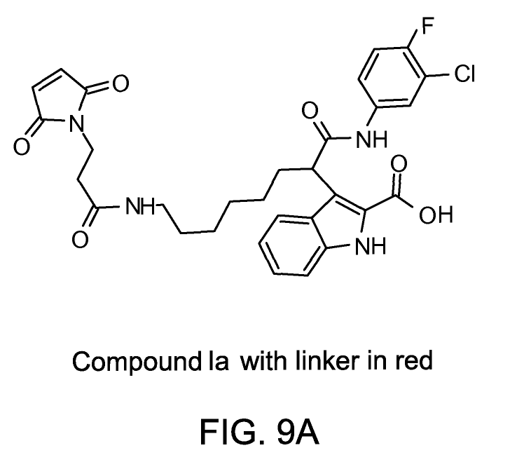

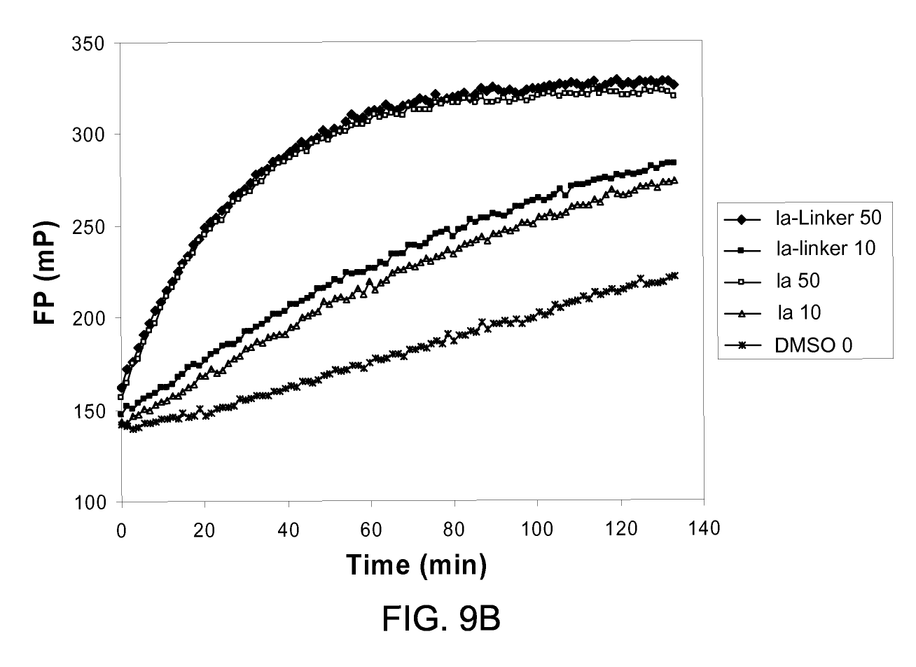

To covalently attach Compound (Ia) to peptides for self-catalyzed loading of such therapeutics to MHC class II molecules, we searched for a site on Compound (Ia) onto which a linker could be attached without loss of activity. In the compound shown in FIG. 8, a linker with a length of four carbons (red lines) was attached to the carbon between the indole ring and the amide. This Compound (Ia)-Linker compound, shown in FIG. 9A, had an activity very similar to Compound (Ia) as shown in the dose-response analysis of FIG. 9B (both compounds at 10 and 50 μM).

### Example 10

### Synthesis of a Compound (Ia)-Maleimide Derivative for Incorporation of Compound (Ia) into Synthetic Peptides

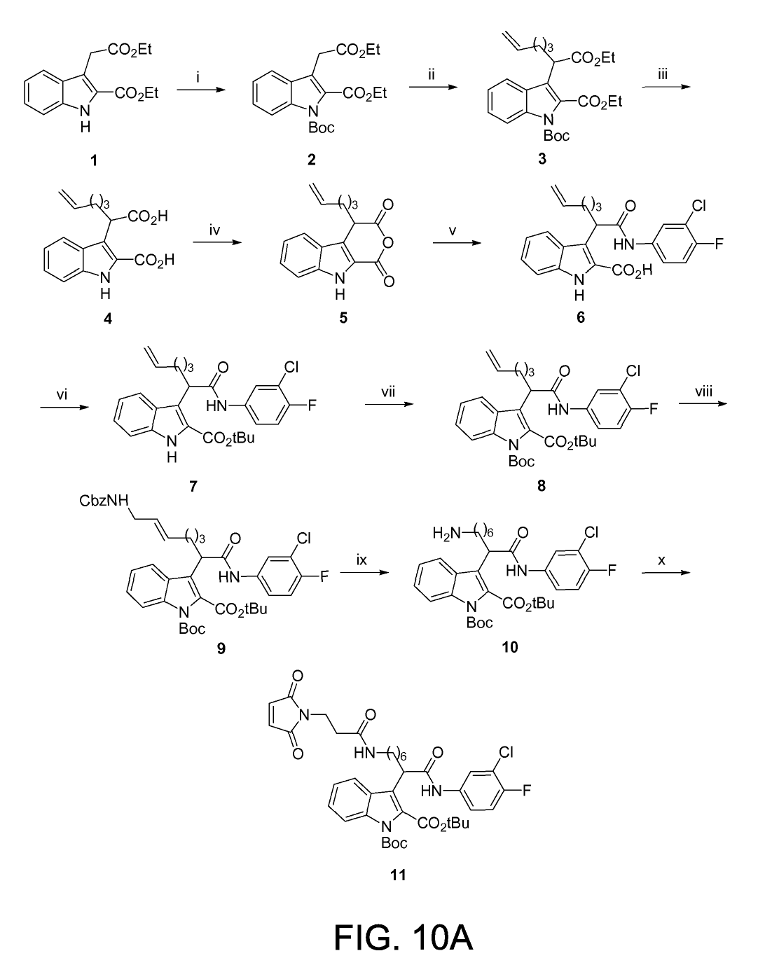

The synthesis of a Compound (Ia) analog is diagramed in FIG. 10. (i) (Boc)₂O, cat. DMAP, DCM, rt, 100%; (ii) NaH, DMF then 5-bromo-1-pentene, 50-68%; (iii) NaOH, EtOH/H₂O, rt, 100%; (iv) DCC, THF, rt; (v) 3-chloro-4-fluoroaniline, toluene, 88% over two steps; (vi) 2-t-butyl-1,3-diisopropylisourea, DCM, rt, 50-80%; (vii) (Boc)₂O, cat. DMAP, DCM, rt, 95%; (viii) cat. (Cy₃P)₂Cl₂Ru═CHPh, DCM, 55%; (ix) H₂ (1 atm), 5% Pd—C, MeOH; (x) 3-maleimidopropionic acid, HBTU, DIPEA, DCM, rt, 72% over two steps.

The synthesis of the Compound (Ia) analog began with protection of the indole nitrogen of 1. Compound 2 was then deprotonated with sodium hydride and then alkylated with 5-bromo-1-pentene to give 3. Hydrolysis of the diester with concomitant loss of the Boc group gave 4. Cyclization With DCC gave anhydride 5, which upon exposure to 3-chloro-4-fluoroaniline regioselectively opened the anhydride yielding the Compound (Ia) derivative 6. The carboxylate was protected as a t-butyl ester using 2-t-butyl-1,3-diisopropylisourea followed by protection of the indole nitrogen with (Boc)₂O to give 8. Cross-metathesis utilizing Grubb's first generation ruthenium catalyst ((Cy₃P)₂Cl₂Ru═CHPh) gave protected amine 9. Hydrogenation removed the Cbz carbamate and reduced the olefin of 9 to give amine 10 without reduction of the indole ring or removal of the halogens. Finally, amide formation with 3-maleimidopropionic acid in the presence of HBTU gave the Compound (Ia) analog 11.

### Example 11

### Activity of Compound (Ia) Covalently Linked to Peptide

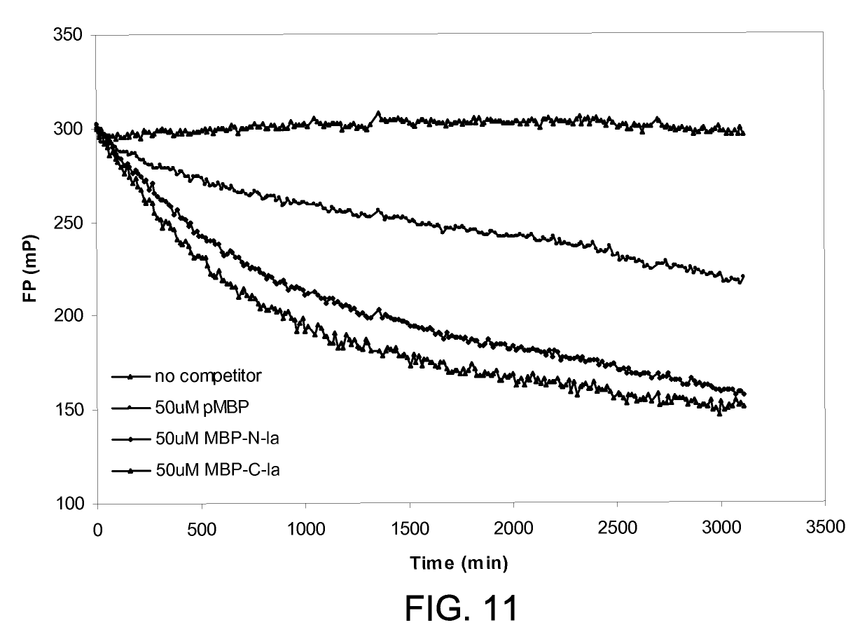

We then tested the concept of “self-catalyzed peptide loading” by synthesizing the MBP peptide with an N-terminal or C-terminal cysteine to which the Compound (Ia)-maleimide was linked. These peptide-Compound (Ia) compounds were purified by reverse-phase HPLC and their identity verified by mass spectrometry. Applicants then examined whether the linked Compound (Ia) group could displace a high affinity peptide from the DR2 binding site. For that purpose, we loaded DR2 (1.5 μM) with the Alexa™-488 labeled MBP peptide (500 nM) at 37° C. for 5 hours. The complex was then diluted to 150 nM DR2/50 nM Alexa™-488 and added to a 384-well plate in a volume of 40 μl. As shown in FIG. 11, either no competitor, MBP peptide without linked Compound (Ia), or MBP peptide with N-terminally or C-terminally linked Compound (Ia) were added to a final concentration of 50 μM and dissociation of the labeled MBP peptide was followed over time. In the absence of competitor peptide, the FP values were stable and slow dissociation was observed in the presence of the MBP competitor peptide without a linked Compound (Ia) group. In contrast, the MBP peptides with a linked Compound (Ia) group were more effective in displacing the labeled peptide from the DR2 binding site. C-terminal attachment of Compound (Ia) was more favorable.

### Example 12

### C-Terminal Attachment of Compound (Ia) Permits Self-Catalyzed Loading of Peptide and Such Conjugates are More Active than Peptide Plus Free Compound (Ia)

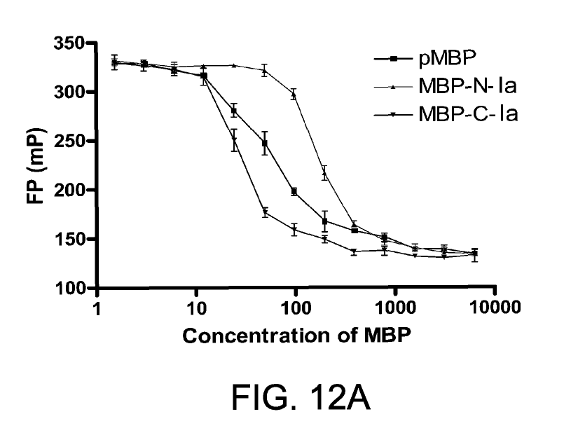

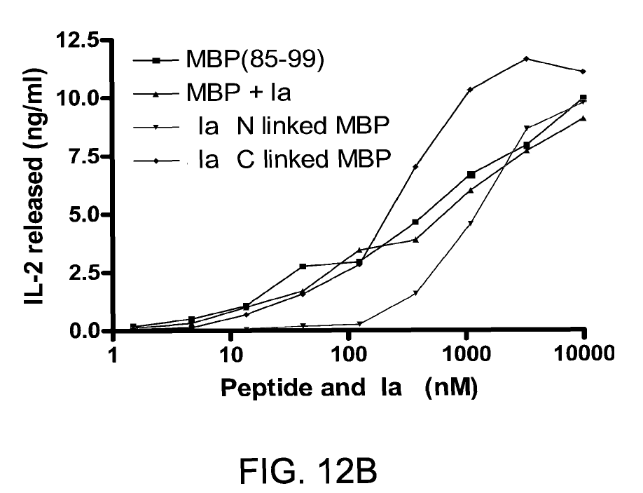

A MBP peptide with a C-terminal Compound (Ia) group (MBP-C-Compound (Ia)) was a more effective competitor than unmodified MBP peptide (FIG. 12A). A peptide with an N-terminal Compound (Ia) group was not stably bound and did not compete as well as unmodified MBP peptide. Competitor peptides were tested over a wide dose range against 10 nM MBP-488 and 100 nM DR2/CLIP in the FP assay.

When tested in a T cell assay, the MBP peptide with the C-terminal Compound (Ia) group induces higher levels of IL-2 production by the DR2/MBP specific T cell hybridoma (FIG. 12B). Peptide presentation of MBP (85-99) to T cell hybridomas by MGAR cells was measured by IL-2 release.

### Example 13

### Identification of Additional High Activity Compounds

Additional compounds were tested in the cellular assay presented in FIG. 7. MGAR cells were incubated with MBP peptide (1 μM) in the presence of different compounds and the amount of DR2 bound peptide was determined by FACS labeling with the MK16 mAb. Tables 1-5 below indicate the compounds tested and their activity levels.

TABLE 1

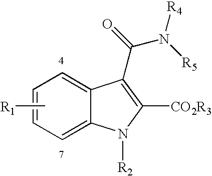

| Structure | R₁ | R₂ | R₃ | R₄ | R₅ | Activity |
|---|---|---|---|---|---|---|
| 00134238 | H | H | H | 3-Cl-4-F-Ph | H | ++ |

TABLE 2

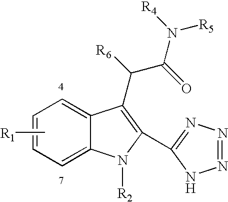

| Structure | R₁ | R₂ | R₄ | R₅ | R₆ | Activity |
|---|---|---|---|---|---|---|
| 00134177 | H | H | 3-Cl-4-F-Ph | H | H | ++ |

TABLE 3

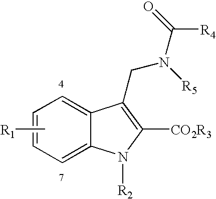

| Structure | R₁ | R₂ | R₃ | R₄ | R₅ | Activity |
|---|---|---|---|---|---|---|
| 00134157 | H | H | H | 3-Cl-4-F-Ph | H | − |

TABLE 4

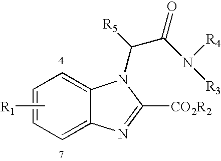

| Structure | R₁ | R₂ | R₃ | R₄ | R₅ | Activity |
|---|---|---|---|---|---|---|
| 00134157 | H | H | H | 3-Cl-4-F-Ph | H | − |

TABLE 5

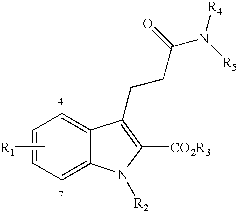

| Structure | R₁ | R₂ | R₃ | R₄ | R₅ | Activity |
|---|---|---|---|---|---|---|
| 00134238 | H | H | H | 3-Cl-4-F-Ph | H | − |

### Example 14

### Identification of Additional High Activity Compounds

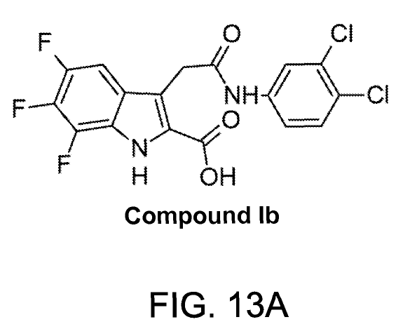

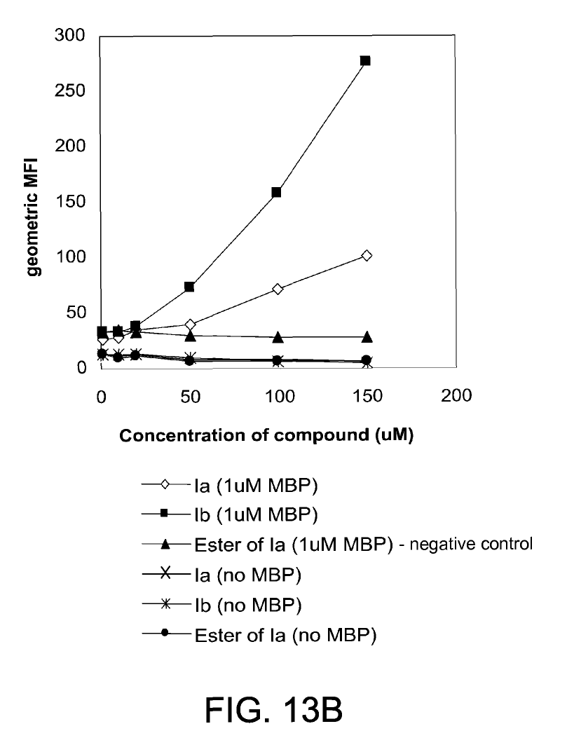

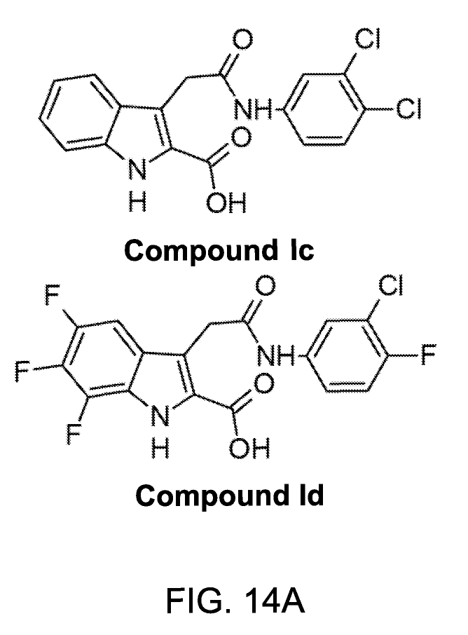

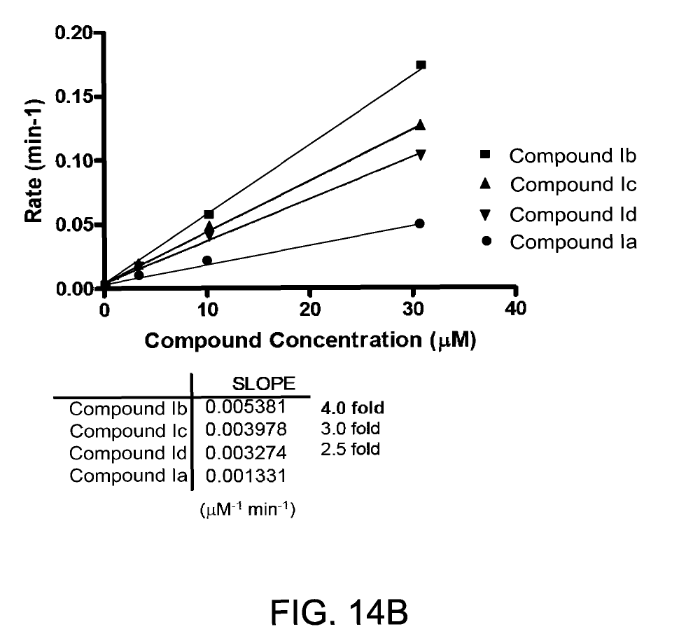

Derivatives of J10 were tested in the cellular assay presented in FIG. 7. MGAR cells were incubated with MBP peptide (1 μM) in the presence of different compounds and the amount of DR2 bound peptide was determined by FACS labeling with the MK16 mAb. Compound (Ib), shown in FIG. 13A, had substantially higher activity than Compound (Ia) as shown in FIG. 13B. In the biochemical assay, Compound (Ib) had 4.2-fold higher activity than Compound (Ia) in catalyzing loading of the MBP peptide to DR2. Compounds Compound (Ic) and Compound (Id), shown in FIG. 14A, also had higher activities than Compound (Ia) as shown in FIG. 14B.

### Example 15

### Identification of Additional High Activity Compounds

Additional compounds were tested in the cellular assay presented in FIG. 7. MGAR cells were incubated with MBP peptide (1 μM) in the presence of different compounds and the amount of DR2 bound peptide was determined by FACS labeling with the MK16 mAb. Table 6 below indicate the compounds tested and their activity levels.

TABLE 6

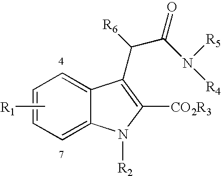

| Struct. | R₁ | R₂ | R₃ | R₄ | R₅ | R₆ | Activity |
|---|---|---|---|---|---|---|---|
| (Ia) | 5-Cl | H | H | 3-Cl-4-F-Ph | H | H | ++ |
| A1 | H | H | H | 3-Cl-4-F-Ph | H | H | +++ |
| A2 | H | H | H | 3-Cl-4-F-Ph | H | (CH₂)₂CH═CH₂ | ++ |
| A3 | H | H | Et | 3-Cl-4-F-Ph | H | H | − |
| A4 | 5-Cl | Me | H | 3-Cl-4-F-Ph | H | H | + |
| A5 | 5-Cl | (CH₂)₅CH═CH₂ | H | 3-Cl-4-F-Ph | H | H | + |
| A6 | H | H | H | 3-Cl-4-F-Ph | H | Cyclobutyl | + |
| A7 | 5-OMe | H | H | 3-Cl-4-F-Ph | H | H | + |
| A8 | 5-F | H | H | 3-Cl-4-F-Ph | H | H | +++ |
| A9 | H | H | H | 3-Cl-4-F-Ph | H | Tetrahydro- | + |
| | | | | | | pyran-4-yl | |
| A10 | H | Me | H | 3-Cl-4-F-Ph | H | H | ++ |
| A11 | 5-Cl | H | H | 4-F-Ph | H | H | + |
| A12 | H | H | H | 3-Cl-Ph | H | H | ++ |
| A13 | H | H | H | 3-MeS-Ph | H | H | + |
| A14 | H | Me | H | 2-F-4-F-Ph | H | H | − |
| A15 | H | Me | H | 2-F-Ph | H | H | − |
| A16 | 5-Cl | H | H | 3-Cl-Ph | Me | H | − |
| A17 | 5-Cl | H | H | 4-Cl-Ph | Me | H | − |
| A18 | H | Me | H | 3-Cl-Ph | Me | H | − |
| A19 | 5-Cl | H | H | 2-Cl-4-F-Ph | H | H | + |
| A20 | 5-Cl | H | H | 2-F-4-F-Ph | H | H | + |
| A21 | H | H | H | 2-F-4-F-Ph | H | H | − |
| A22 | H | H | H | 2-MeO-4-MeO-Ph | H | H | − |
| A23 | H | H | H | 2-CO₂Me-Ph | H | H | − |
| A24 | H | H | H | 5-Cl-pyridine-2-yl | H | H | ++ |
| A25 | 5-Cl | H | H | 5-Me-isoxazol-3-yl | H | H | ++ |
| A26 | 5-Cl | H | H | Cyclohexyl | H | H | − |
| A27 | 5-Cl | H | H | 5-Cl-pyridine-2-yl | H | H | ++ |
| A28 | 5-Cl | H | H | Pyridine-3-yl | H | H | + |
| A29 | 5-Me | Me | H | 5-Me-isoxazol-3-yl | H | H | − |
| A30 | 7-F | H | H | 3-Cl-4-F-Ph | H | H | +++ |
| (Id) | 5-F-6-F-7-F | H | H | 3-Cl-4-F-Ph | H | H | +++++ |
| A31 | H | H | H | 3-F-4-F-Ph | H | H | + |
| A32 | H | H | H | 3-F-4-F-5-F-Ph | H | H | ++ |
| A33 | H | H | H | 3-Cl-5-Cl-Ph | H | H | + |
| (Ic) | H | H | H | 3-Cl-4-Cl-Ph | H | H | +++++ |
| A36 | H | H | H | 2-Pyrimidinyl | H | H | − |
| A37 | H | H | H | 2-Thiazolyl | H | H | − |
| A38 | H | H | H | Pyrazinyl | H | H | − |
| A39 | 5-F-7-F | H | H | 3-Cl-4-F-Ph | H | H | ++++ |
| A40 | 4-F-7-F | H | H | 3-Cl-4-F-Ph | H | H | +++ |
| A41 | H | H | H | 3-Cl-4-Cl-5-Cl-Ph | H | H | +++ |
| A42 | H | H | H | Ph | H | H | − |
| (Ib) | 5-F-6-F-7-F | H | H | 3-Cl-4-Cl-Ph | H | H | ++++++ |
| A43 | 6-F-7-F | H | H | 3-Cl-4-F-Ph | H | H | ++++ |
| A44 | H | H | H | 3-Cl-4-Br-Ph | H | H | +++++ |
| A45 | H | H | H | 4-Cl-Ph | H | H | ++ |
| A46 | H | H | H | 4-Br-Ph | H | H | − |
| A47 | H | H | H | 3-F-4-Cl-Ph | H | H | +++ |
| A48 | 7-Cl | H | H | 3-Cl-4-F-Ph | H | H | ++ |
| A49 | H | H | H | 3-Cl-4-F-Ph | H | ═O | − |
| A50 | H | H | H | 2-Naphthyl | H | H | + |
| Structure | R₁ | R₂ | R₃ | R₄ | R₅ | R₆ | Activity |
| A51 | 5-Cl | H | H | Pyridine-2-yl-methyl | H | H | − |
| A52 | H | Me | H | sec-Butyl | H | H | − |
| A53 | H | Me | H | (Tetrahydrofuran-2yl)-methyl | H | H | − |
| A54 | H | Me | H | Isopropyl | H | H | − |
| A55 | H | Me | H | Cycloheptyl | H | H | − |
| A56 | H | H | H | Cyclopentyl | H | H | − |
| A57 | 5-Cl | H | H | Cycloheptyl | H | H | − |
| A58 | H | H | H | (Furan-2-yl)-methyl | H | H | − |
| A59 | H | H | H | Cyclopropyl | H | H | − |
| A60 | H | Me | H | (2,3-Dihydrobenzo[1,4]dioxin- | H | H | − |
| | | | | 2-yl)-methyl | | | |
| A61 | H | Me | H | 3-(4-Methylpiperidin-1-yl)- | H | H | − |
| | | | | propyl | | | |
| A62 | H | H | H | Isopropyl | H | H | − |
| A63 | 5-Cl | H | H | Thiophen-2-yl-methyl | H | H | − |
| A64 | 5-Cl | H | H | (5-Methylfuran-2-yl)-methyl | H | H | − |
| A65 | 5-Cl | H | H | Cyclopentyl | H | H | − |
| A66 | H | H | H | 3-Cl-4-F-benzyl | H | H | − |
| A67 | H | H | H | 4-Cl-pyridine-2-yl | H | H | − |
| A68 | H | H | H | 1,3,4-Thiadiazolyl | H | H | − |
| A69 | H | H | H | 2,3-Dichloro-pyridine-5-yl | H | H | ++ |
| A70 | H | H | H | 3-CN-4-F-Ph | H | H | ++ |
| A-71 | H | H | H | 3-Cl-4-CN-Ph | H | H | +++++ |
| A-72 | H | H | H | 3-Cl-4-CF₃-Ph | H | H | ++ |
| A-73 | H | H | H | 3-NO₂-4-NO₂-Ph | H | H | ++++ |
| A-74 | H | H | H | 3-CN-4-CN-Ph | H | H | ++ |
| A-75 | H | H | H | 4-Cl-3-CF₃-Ph | H | H | +++ |
| A-76 | 6-F-5-Me | H | H | 3-Cl-4-F-Ph | H | H | + |
| A-77 | 6-F-7-F | H | H | 3-Cl-4-Cl-Ph | H | (CH₂)₃CH═CH₂ | − |
| A-78 | H | H | H | 4-Cl-3-Me-Ph | H | H | ++ |
| A-79 | 6-F-5-Me | H | H | 3-Cl-4-Cl-Ph | H | H | ++ |
| A-80 | 6-F-7-F | H | H | 3-Cl-4-CN-Ph | H | H | ++++ |
| A-81 | 5-F-6-F-7-F | H | H | 3-Cl-4-CN-Ph | H | H | ++++ |
| A-82 | H | H | H | 3-Me-4-Me-Ph | H | H | − |
| A-83 | H | H | H | 3-Cl-4-Cl-Ph | H | (CH₂)₃CH═CH₂ | + |
| A-84 | H | H | H | 3-Cl-4-CN-Ph | H | (CH₂)₃CH═CH₂ | + |
| A-85 | 5-F-6-F-7-F | (CH₂)₃CH═CH₂ | H | 3-Cl-4-Cl-Ph | H | H | − |
| A-86 | 5-F-6-F-7-F | H | H | 4-Cl-3-Me-Ph | H | H | ++ |
| A-87 | 5-F-6-F-7-F | H | H | 3-Cl-4-Cl-Ph | H | (CH₂)₃CH═CH₂ | + |

### Example 16

### Use of DM Mimics for Immunization with Viral and Tumor Peptides

HLA-DR4 transgenic mice (DRA, DRB1*0401) are used to examine whether Compound (Ib), Compound (Ia) and the other compounds enhance the CD4 T cell response following immunization with viral and tumor peptides. Mice are immunized with peptides from two human pathogens (HIV and HCV) as well as peptides from two human tumor antigens (annexin V and gp100, identified in human melanomas). Applicants have previously performed binding studies with these peptides and shown that they bind with high affinity to DR4. The T cell response is analyzed in two different assays. First, the frequency of peptide-specific T cells is analyzed in draining lymph nodes with DR4/peptide tetramers. This approach provides a quantitative readout of the induced T cell response. Second, the cytokine production profile of these T cells is determined following a 48 hour in vitro re-stimulation with the relevant peptide. The cytometric bead array technique is utilized for comprehensive definition of the cytokine repertoire. Either Compound (Ia) is co-administered with the peptide of interest in the adjuvant or peptides are utilized with a covalently linked Compound (Ia) group (or other groups, depending on which one is being tested). Based on the in vitro studies, substantial enhancement in the CD4 T cell response that is induced is expected. These experiments are expected to establish the in vivo efficacy of Compound (Ia) and to establish appropriate dosing and administration procedures with rapid, quantitative readouts. The immunization approach also enables a comparison of the efficacy of different Compound (Ia) derivatives.

### Example 17

### Tolerance Induction with Peptides from Self-Antigens

We will utilize two transgenic mouse models to determine whether Compound (Ia) enhances the efficacy of peptides for the treatment of autoimmune diseases. DR4 transgenic mice can be utilized as animal models of MS and rheumatoid arthritis by immunization with a myelin peptide (derived from proteolipid protein, PLP 176-192) or a type II collagen peptide. DR2 transgenic mice that also express a human TCR (specific for DR2/MBP 85-99, isolated from a patient with MS) can be used, and these mice develop spontaneous inflammation and demyelination in the CNS. Applicants will compare the efficacy of tolerance induction with unmodified and Compound (Ia)-linked peptides

The in vitro experiments have shown that a 10-fold lower dose is required in the presence of Compound (Ia) for an equivalent level of peptide presentation. Applicants will therefore determine the degree of protection relative to peptide dose for unmodified and Compound (Ia)-linked peptides. Enhanced presentation of peptides is expected to not only improve the dose response relationship but also to induce a higher frequency of T cells that produce protective cytokines. Applicants will examine the cytokine profile (in particular IL-4, IL-10, TGFβ) of lymph node cells following peptide administration using standard methods.

### Example 18

### Toxicology and Pharmacokinetics

Understanding Compound (Ib) toxicity will help facilitate the design of pharmacokinetics studies and, ultimately, in vivo efficacy studies. Acute maximum tolerated dose (MTD) studies are to be used for chronic dose ranging studies. Three groups of mice (n=6) will be dosed at MTD/3, MTD/10 and MTD/30 daily for 10 days. A dose is judged non-toxic and suitable for pharmacokinetics and efficacy studies based upon an assessment of the animal's general health, behavior and weight loss. Brains are to be harvested and the compound's ability to cross the blood brain barrier assessed, both in normal mice and mice with EAE because the blood brain barrier is permeable even to large proteins at sites of inflammation. After dosing, T cell populations are to be tested to determine the relative ratios of T cells (CD4/CD8 T cells) and other white blood cells in lymph nodes, spleen and peripheral blood. Organs will be checked for aberrant lymphocyte infiltrations. Compounds will be administered either i.v. or i.p. to mice at a dose of 10 mg/kg or a dose determined by animal toxicity studies outlined above. Multiple samples of blood will be collected over a period of 24 hours and analyzed for the parent compound. With the plasma half-life, a bioavailability calculation will determine the dosing for animal efficacy studies described above.

### Example 19

### General procedure for the preparation of 2-carboxy-3-indoleacetamide

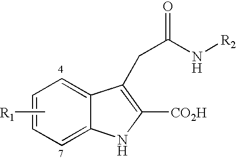

This general procedure follows the general methodology described by Gray et al. (see: J. Med. Chem., 1991, 34 (4), 1283). Aniline (27 mmol) was dissolved in 37% HCl (14 mL) and H₂O (26 mL) at 0° C. A solution of NaNO₂ (2.04 g, 29.6 mmol) in H₂O (12 mL) was added dropwise at 0° C. The resulting solution was then added slowly at 0° C. to a mixture of diethyl 2-acetylglutarate (6.22 g, 27 mmol), 6N NaOH (24 mL), and ethanol (30 mL). The mixture was stirred for 3 h (0° C. to rt) and extracted with ethyl acetate (200 mL). The red organic layer was dried over Na₂SO₄ and concentrated to give a dark red mixture, which was dissolved in 50 mL of absolute ethanol and treated with concentrated H₂SO₄ (10 mL). After refluxing (120° C.) for 2 d, the mixture was cooled to room temperature, quenched with saturated NaHCO₃, and extracted with ethyl acetate. The organic layer was washed with brine, dried over anhydrous Na₂SO₄, filtered and concentrated. The residue was purified by chromatography on silica gel using hexane/EtOAc (85:15) to give a product as a brown solid, which was further purified by washing with hexane to give an off-white solid (5-30%).

The diester (1.0 mmol) was dissolved in absolute ethanol (9 mL) and treated with 12 N NaOH solution (1 mL). The mixture was stirred at room temperature for 3-16 h before being acidified with 1 N HCl, extracted with ethyl acetate. The organic layer was washed with brine, dried over anhydrous Na₂SO₄, filtered and concentrated to give a solid.

The diacid was dissolved in dry THF (40 mL) and treated with dicyclohexylcarbodiimide (DCC, 206 mg, 1.0 mmol) at room temperature. After stirring for 3-5 h, the mixture was concentrated and mixed with ethyl acetate. The white solid was removed by filtration and washed with ethyl acetate. The filtrate was concentrated to give the desired anhydride.

A mixture of the anhydride (0.4 mmol), an aniline or amine (0.6 mmol) and toluene was heated to 110° C. and stirred for 2-16 h before cooled down to room temperature. The solid was collected by filtration and washed with dichloromethane to give a pure product. An additional amount of product was recovered from the filtrate by chromatography on silica gel using CH₂Cl₂/MeOH (92:8) as eluent (20-90% yield over three steps).

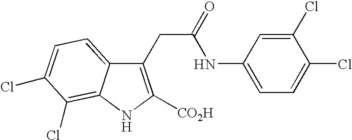

¹H NMR (400 MHz, DMSO-d6) δ 12.92 (s, 1H), 10.45 (s, 1H), 7.94 (d, J=2.0 Hz, 1H), 7.68 (d, J=8.4 Hz, 1H), 7.55-7.46 (m, 2H), 7.26 (d, J=8.4 Hz, 1H), 4.18 (s, 2H).

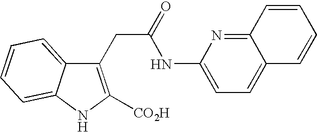

¹H NMR (500 MHz, DMSO-d6) δ 11.61 (s, 1H), 10.61 (s, 1H), 9.13 (s, 1H), 8.39 (s, 1H), 8.04 (d, J=8.5 Hz, 1H), 7.82 (d, J=8.0 Hz, 1H), 7.70-7.66 (m, 2H), 7.53-7.49 (m, 1H), 7.42 (d, J=8.5 Hz, 1H), 7.25 (t, J=8.0 Hz, 1H), 7.25 (t, J=8.5 Hz, 1H), 4.28 (s, 2H).

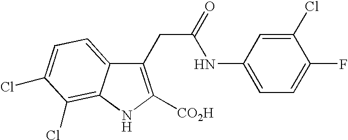

¹H NMR (400 MHz, DMSO-d6) δ 11.92 (s, 1H), 10.33 (s, 1H), 7.87 (dd, J=6.8, 2.4 Hz, 1H), 7.67 (d, J=8.4 Hz, 1H), 7.47-7.43 (m, 1H), 7.34 (t, J=8.8 Hz, 1H), 7.26 (d, J=8.4 Hz, 1H), 4.18 (s, 2H).

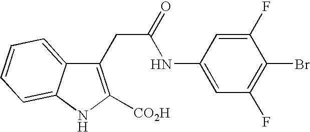

¹H NMR (500 MHz, DMSO-d6) δ 11.62 (s, 1H), 10.68 (s, 1H), 7.87 (d, J=8.5 Hz, 1H), 7.49 (d, J=8.0 Hz, 1H), 7.42 (d, J=8.0 Hz, 1H), 7.25 (t, J=8.8 Hz, 1H), 7.05 (t, J=8.8 Hz, 1H), 4.20 (s, 2H).

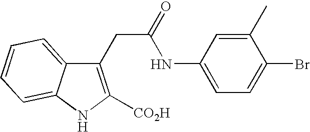

¹H NMR (500 MHz, DMSO-d6) δ 11.60 (s, 1H), 10.66 (s, 1H), 8.38 (s, 1H), 8.04 (s, 1H), 7.64 (d, J=8.5 Hz, 1H), 7.49 (d, J=8.5 Hz, 1H), 7.24 (t, J=8.0 Hz, 1H), 7.05 (t, J=8.0 Hz, 1H), 4.22 (s, 2H), 2.30 (s, 3H).

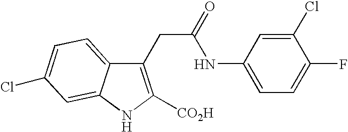

¹H NMR (500 MHz, DMSO-d6) δ 11.75 (s, 1H), 10.36 (s, 1H), 7.87 (dd, J=7.0, 3.0 Hz, 1H), 7.49 (d, J=9.0 Hz, 1H), 7.49-7.46 (m, 1H), 7.42 (d, J=2.0 Hz, 1H), 7.36 (t, J=9.0 Hz, 1H), 7.05 (dd, J=8.5, 2.0 Hz, 1H), 4.17 (s, 2H).

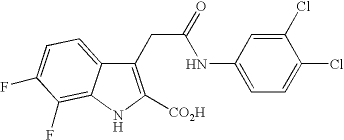

¹H NMR (500 MHz, DMSO-d6) δ 12.31 (s, 1H), 10.43 (s, 1H), 7.97 (d, J=2.0 Hz, 1H), 7.56 (d, J=8.5 Hz, 1H), 7.52-7.48 (m, 2H), 7.14-7.09 (m, 1H), 4.19 (s, 2H).

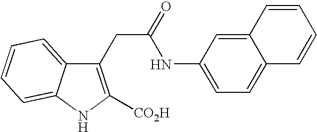

¹H NMR (500 MHz, DMSO-d6) δ 13.10 (br.s, 1H), 11.59 (s, 1H), 10.35 (s, 1H), 8.27 (d, J=2.0 Hz, 1H), 7.85 (d, J=9.0 Hz, 1H), 7.82 (d, J=8.0 Hz, 1H), 7.75 (d, J=8.0 Hz, 1H), 7.70 (d, J=8.0 Hz, 1H), 7.61 (dd, J=9.0, 2.0 Hz, 1H), 7.46-7.36 (m, 3H), 7.25 (t, J=8.0 Hz, 1H), 7.25 (t, J=8.0 Hz, 1H), 4.24 (s, 2H).

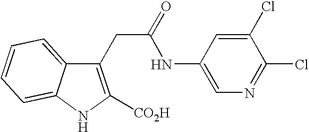

¹H NMR (400 MHz, DMSO-d6) δ 13.06 (br.s, 1H), 11.60 (s, 1H), 10.70 (s, 1H), 8.52 (d, J=2.4 Hz, 1H), 8.36 (d, J=2.4 Hz, 1H), 7.64 (d, J=8.0 Hz, 1H), 7.41 (d, J=8.0 Hz, 1H), 7.23 (t, J=8.0 Hz, 1H), 7.04 (t, J=8.0 Hz, 1H), 4.21 (s, 2H).

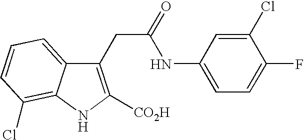

¹H NMR (500 MHz, DMSO-d6) δ 13.27 (br.s, 1H), 11.72 (s, 1H), 10.37 (s, 1H), 7.89 (dd, J=7.0, 2.5 Hz, 1H), 7.66 (d, J=8.5 Hz, 1H), 7.49-7.46 (m, 1H), 7.36 (t, J=9.0 Hz, 1H), 7.34 (d, J=7.5 Hz, 1H), 7.08 (t, J=7.5 Hz, 1H), 4.17 (s, 2H).

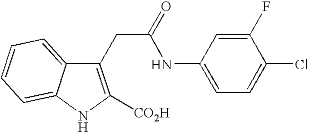

¹H NMR (500 MHz, DMSO-d6) δ 13.05 (br.s, 1H), 11.60 (s, 1H), 10.50 (s, 1H), 7.77 (dd, J=7.0, 2.0 Hz, 1H), 7.65 (d, J=8.5 Hz, 1H), 7.50 (t, J=8.5 Hz, 1H), 7.41 (d, J=8.0 Hz, 1H), 7.34 (dd, J=8.5, 2.0 Hz, 1H), 7.24 (t, J=8.0 Hz, 1H), 7.08 (t, J=7.5 Hz, 1H), 4.18 (s, 2H).

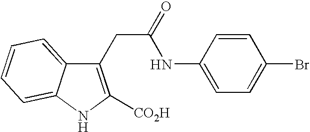

¹H NMR (500 MHz, DMSO-d6) δ 13.06 (br.s, 1H), 11.58 (s, 1H), 10.26 (s, 1H), 7.65 (d, J=8.5 Hz, 1H), 7.56 (d, J=8.5 Hz, 2H), 7.46 (d, J=8.5 Hz, 2H), 7.41 (d, J=8.0 Hz, 1H), 7.24 (t, J=8.0 Hz, 1H), 7.04 (t, J=8.0 Hz, 1H), 4.17 (s, 2H).

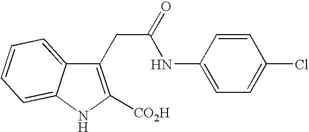

¹H NMR (500 MHz, DMSO-d6) δ 13.06 (br.s, 1H), 11.58 (s, 1H), 10.27 (s, 1H), 7.65 (d, J=8.0 Hz, 1H), 7.61 (d, J=8.5 Hz, 2H), 7.41 (d, J=8.0 Hz, 1H), 7.34 (d, J=8.5 Hz, 2H), 7.24 (t, J=7.0 Hz, 1H), 7.04 (1, J=7.0 Hz, 1H), 4.17 (s, 2H).

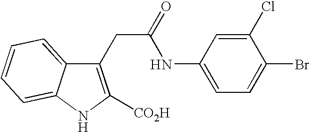

¹H NMR (500 MHz, DMSO-d6) δ 13.07 (br.s, 1H), 11.60 (s, 1H), 10.47 (s, 1H), 7.97 (d, J=2.0 Hz, 1H), 7.67 (d, J=9.0 Hz, 2H), 7.65 (d, J=8.0 Hz, 1H), 7.44-7.41 (m, 2H), 7.24 (t, J=8.0 Hz, 1H), 7.05 (t, J=7.5 Hz, 1H), 4.18 (s, 2H).

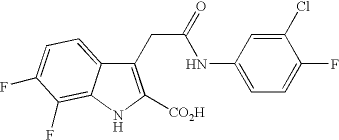

¹H NMR (500 MHz, DMSO-d6) δ 13.35 (br.s, 1H), 12.24 (s, 1H), 10.57 (s, 1H), 7.88 (dd, J=7.0, 2.5 Hz, 1H), 7.50-7.485 (m, 2H), 7.35 (t, J=9.0 Hz, 1H), 7.13-7.08 (m, 1H), 4.15 (s, 2H).

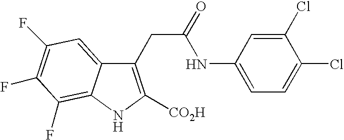

¹H NMR (500 MHz, DMSO-d6) δ 13.39 (br.s, 1H), 12.46 (s, 1H), 10.42 (s, 1H), 7.97 (d, J=2.0 Hz, 1H), 7.67-7.64 (m, 1H), 7.56 (d, J=8.5 Hz, 1H), 7.65 (dd, J=9.0, 2.5 Hz, 1H), 4.16 (s, 2H).

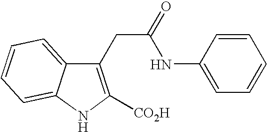

¹H NMR (500 MHz, DMSO-d6) δ 13.08 (br.s, 1H), 11.57 (s, 1H), 10.11 (s, 1H), 7.66 (d, J=8.5 Hz, 1H), 7.58 (d, J=8.5 Hz, 2H), 7.41 (d, J=8.5 Hz, 1H), 7.28 (1, J=8.0 Hz, 2H), 7.24 (t, J=8.0 Hz, 1H), 7.06-7.00 (m, 2H), 4.17 (s, 2H).

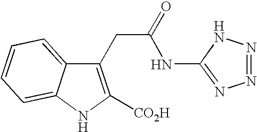

¹H NMR (500 MHz, DMSO-d6) δ 13.08 (br.s, 1H), 12.21 (s, 1H), 11.65 (s, 1H), 7.67 (d, J=8.0 Hz, 1H), 7.58 (d, J=8.0 Hz, 2H), 7.25 (t, J=8.0 Hz, 2H), 7.06 (t, J=8.0 Hz, 1H), 4.30 (s, 2H).

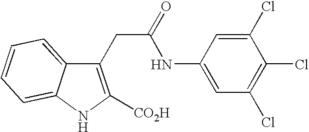

¹H NMR (500 MHz, DMSO-d6) δ 13.07 (br.s, 1H), 11.62 (s, 1H), 10.53 (s, 1H), 7.87 (s, 2H), 7.65 (d, J=8.0 Hz, 1H), 7.42 (d, J=8.0 Hz, 1H), 7.25 (t, J=8.0 Hz, 1H), 7.05 (t, J=8.0 Hz, 1H), 4.19 (s, 2H).

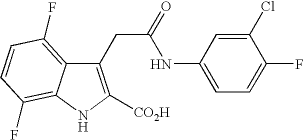

¹H NMR (500 MHz, DMSO-d6) δ 13.44 (br.s, 1H), 12.39 (s, 1H), 10.30 (s, 1H), 7.88 (dd, J=8.0, 2.0 Hz, 1H), 7.48-7.44 (m, 1H), 7.36 (t, J=9.0 Hz, 1H), 7.06-7.01 (m, 1H), 6.77-6.73 (m, 1H), 4.27 (s, 2H).

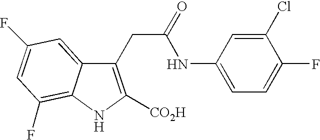

¹H NMR (500 MHz, DMSO-d6) δ 12.20 (s, 1H), 10.40 (s, 1H), 7.87 (dd, J=6.5, 2.5 Hz, 1H), 7.47-7.44 (m, 1H), 7.36-7.32 (m, 2H), 7.15-7.11 (m, 1H), 4.13 (s, 2H).

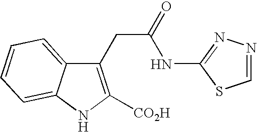

¹H NMR (500 MHz, DMSO-d6) δ 12.79 (s, 1H), 11.66 (s, 1H), 9.13 (s, 1H), 7.65 (d, J=8.0 Hz, 1H), 7.42 (d, J=8.0 Hz, 1H), 7.25 (t, J=8.0 Hz, 1H), 7.05 (t, J=8.0 Hz, 1H), 4.34 (s, 2H).

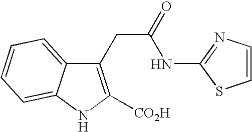

¹H NMR (500 MHz, DMSO-d6) δ 13.03 (br.s, 1H), 12.29 (s, 1H), 11.63 (s, 1H), 7.64 (d, J=8.5 Hz, 1H), 7.46 (d, J=3.5 Hz, 1H), 7.42 (d, J=8.5 Hz, 1H), 7.25 (t, J=8.0 Hz, 1H), 7.17 (d, J=3.5 Hz, 1H), 7.05 (t, J=8.0 Hz, 1H), 4.28 (s, 2H).

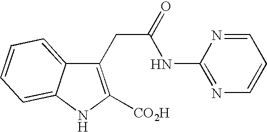

¹H NMR (500 MHz, DMSO-d6) δ 11.58 (s, 1H), 10.65 (s, 1H), 8.64 (d, J=5.0 Hz, 2H), 7.63 (d, J=7.5 Hz, 1H), 7.42 (d, J=7.5 Hz, 1H), 7.24 (t, J=7.5 Hz, 1H), 7.16 (d, J=5.0 Hz, 1H), 7.05 (t, J=7.5 Hz, 1H), 4.31 (s, 2H).

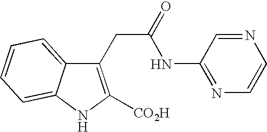

¹H NMR (500 MHz, DMSO-d6) δ 11.61 (s, 1H), 10.92 (s, 1H), 9.26 (s, 1H), 8.40 (d, J=2.5 Hz, 1H), 8.34 (d, J=2.5 Hz, 1H), 7.66 (d, J=8.0 Hz, 1H), 7.42 (d, J=8.0 Hz, 1H), 7.24 (t, J=7.5 Hz, 1H), 7.05 (t, J=7.5 Hz, 1H), 4.28 (s, 2H).

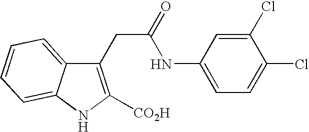

¹H NMR (500 MHz, DMSO-d6) δ 13.05 (br.s, 1H), 11.58 (s, 1H), 10.43 (s, 1H), 7.97 (s, 1H), 7.65 (d, J=8.0 Hz, 1H), 7.56-7.49 (m, 2H), 7.42 (d, J=8.0 Hz, 1H), 7.24 (t, J=7.5 Hz, 1H), 7.04 (t, J=7.5 Hz, 1H), 4.18 (s, 2H).

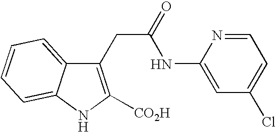

¹H NMR (500 MHz, DMSO-d6) δ 11.61 (s, 1H), 10.84 (s, 1H), 8.30 (d, J=5.0 Hz, 2H), 8.10 (d, J=1.5 Hz, 1H), 7.65 (d, J=8.0 Hz, 1H), 7.42 (d, J=8.5 Hz, 1H), 7.26-7.22 (m, 2H), 7.05 (t, J=8.0 Hz, 1H), 4.25 (s, 2H).

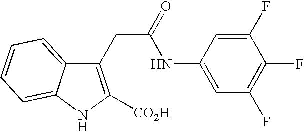

¹H NMR (500 MHz, DMSO-d6) δ 13.09 (br.s, 1H), 11.60 (s, 1H), 10.48 (s, 1H), 7.64 (d, J=8.5 Hz, 1H), 7.50 (dd, J=10.5, 6.5 Hz, 2H), 7.42 (d, J=8.0 Hz, 1H), 7.25 (t, J=8.0 Hz, 1H), 7.05 (t, J=8.0 Hz, 1H), 4.18 (s, 2H).

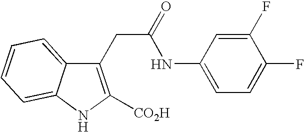

¹H NMR (500 MHz, DMSO-d6) δ 13.04 (br.s, 1H), 11.59 (s, 1H), 10.35 (s, 1H), 7.79-7.74 (m, 1H), 7.65 (d, J=8.0 Hz, 1H), 7.42 (d, J=8.0 Hz, 1H), 7.40-7.29 (m, 2H), 7.24 (t, J=8.0 Hz, 1H), 7.05 (t, J=8.0 Hz, 1H), 4.17 (s, 2H).

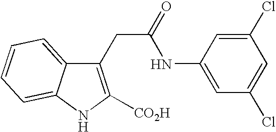

¹H NMR (500 MHz, DMSO-d6) δ 13.05 (br.s, 1H), 11.59 (s, 1H), 10.44 (s, 1H), 7.66-7.64 (m, 3H), 7.42 (d, J=8.0 Hz, 1H), 7.26-7.23 (m, 2H), 7.05 (t, J=8.0 Hz, 1H), 4.18 (s, 2H).

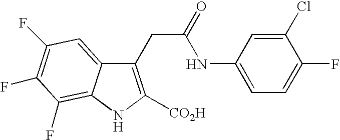

¹H NMR (500 MHz, DMSO-d6) δ 13.34 (br.s, 1H), 12.44 (s, 1H), 10.35 (s, 1H), 7.88 (dd, J=7.0, 2.5 Hz, 1H), 7.66-7.62 (m, 1H), 7.49-7.45 (m, 1H), 7.36 (t, J=9.0 Hz, 1H), 4.15 (s, 2H).

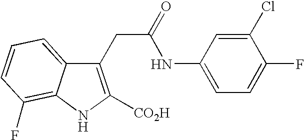

¹H NMR (500 MHz, DMSO-d6) δ 12.04 (s, 1H), 10.40 (s, 1H), 7.87 (dd, J=6.5, 2.5 Hz, 1H), 7.48-7.44 (m, 2H), 7.34 (t, J=9.0 Hz, 1H), 7.08-7.00 (m, 2H), 4.16 (s, 2H).

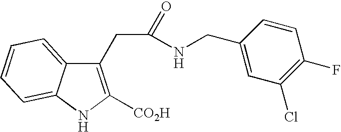

¹H NMR (500 MHz, DMSO-d6) δ 11.53 (s, 1H), 8.34 (s, 1H), 7.59 (d, J=8.5 Hz, 1H), 7.36 (dd, J=7.0, 2.0 Hz, 1H), 7.30 (t, J=9.0 Hz, 1H), 7.24-7.20 (m, 1H), 7.02 (t, J=7.5 Hz, 1H), 4.22 (d, J=6.0 Hz, 2H), 4.01 (s, 2H).

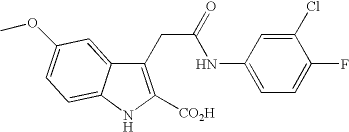

¹H NMR (500 MHz, DMSO-d6) δ 11.45 (s, 1H), 10.30 (s, 1H), 7.91 (dd, J=7.0, 2.05 Hz, 1H), 7.50-7.47 (m, 1H), 7.36 (t, J=9.0 Hz, 1H), 7.31 (d, J=9.0 Hz, 1H), 7.12 (d, J=2.5 Hz, 1H), 6.91 (dd, J=9.0, 2.5 Hz, 1H), 4.13 (s, 2H), 3.74 (s, 3H).

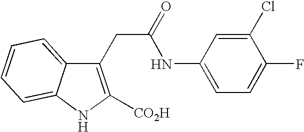

¹H NMR (500 MHz, DMSO-d6) δ 11.47 (s, 1H), 10.41 (s, 1H), 7.87-7.76 (m, 1H), 7.61 (d, J=8.0 Hz, 1H), 7.46-7.44 (m, 1H), 7.40 (d, J=8.0 Hz, 1H), 7.21-7.18 (m, 2H), 7.02 (t, J=7.5 Hz, 1H), 4.14 (s, 2H).

### Example 20

### Synthesis of Compound (Ia)-Maleimide Derivative

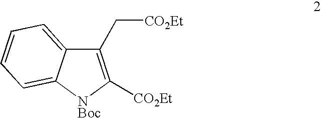

A solution of 3-ethyoxycarbonylmethyl-1H-indole-2-carboxylic acid ethyl ester, 1, (413 mg, 1.5 mmol), di-tert-butyl dicarbonate (436 mg, 2.0 mmol), 4-(dimethylamine) pyridine (36 mg, 0.3 mmol) in CH₂Cl₂ (30 mL) was stirred for 2 hours at room temperature before removal of the solvent. The residue was quenched with 1 N HCl and extracted with ethyl acetate. The organic layer was washed with brine and concentrated. The crude mixture was purified by chromatography on silica gel using hexane/EtOAc (85:15) to give 2 as pale yellow oil (560 mg, 100%). ¹H NMR (400 MHz, CDCl₃) δ 8.12 (d, J=8.4 Hz, 1H), (d, J=8.0 Hz, 1H), 7.41 (t, J=8.4 Hz, 1H), 7.30 (t, J=8.0 Hz, 1H), 4.40 (q, J=7.2 Hz, 2H), 4.13 (q, J=7.2 Hz, 2H), 3.91 (s, 2H), 1.63 (s, 9H), 1.40 (t, J=7.2 Hz, 3H), 1.22 (t, J=7.2 Hz, 3H).

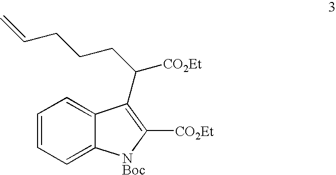

To a solution of compound 2 (1130 mg, 3.0 mmol) and NaH (60%, 140 mg, 3.5 mmol) in DMF (30 mL) at 0° C. was added 5-bromo-1-pentene (447 mg, 3.0 mmol). The mixture was allowed to warm up to room temperature and stirred overnight, quenched with 1N HCl and extracted with ethyl acetate. The organic solution was washed with brine and concentrated. The residue was purified by chromatography on silica gel using hexane/EtOAC (90:10 to 75:25) to afford 3 (600 mg) as light yellow oil and 273 mg of recovered starting material 2. ¹H NMR (500 MHz, CDCl₃) δ 8.08 (d, J=8.0 Hz, 1H), 7.73 (d, J=8.0 Hz, 1H), 7.38 (t, J=8.0 Hz, 1H), 7.24 (t, J=8.0 Hz, 1H), 5.78-5.69 (m, 1H), 4.97-4.89 (m, 2H), 4.43-4.37 (m, 2H), 4.15-4.03 (m, 3H), 2.28-2.21 (m, 1H), 2.08-1.93 (m, 3H), 1.63 (s, 9H), 1.39 (t, J=7.0 Hz, 3H), 1.47-1.26 (m, 2H), 1.14 (t, J=7.0 Hz, 3H).

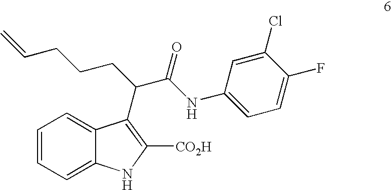

Compound 6 was prepared from 3 by following the general procedure for the preparation of 2-carboxy-3-indoleacetamide. ¹H NMR (500 MHz, DMSO-d6) δ 11.47 (s, 1H), 10.33 (br.s, 1H), 7.86 (dd, J=7.0, 2.5 Hz, 1H), 7.75 (d, J=8.0 Hz, 1H), 7.49-7.45 (m, 1H), 7.38 (d, J=8.5 Hz, 1H), 7.29 (t, J=9.0 Hz, 1H), 7.16 (t, J=7.0 Hz, 1H), 6.97 (t, J=8.0 Hz, 1H), 5.78-5.69 (m, 1H), 4.97-4.88 (m, 2H), 4.79-4.73 (m, 1H), 2.28-2.20 (m, 1H), 2.06-1.99 (m, 2H), 1.91-1.84 (m, 1H), 1.40-1.34 (m, 1H), 1.28-1.20 (m, 1H).

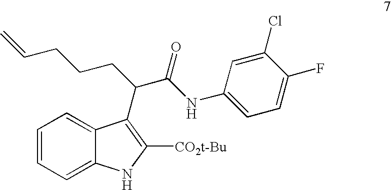

A mixture of 6 (70 mg, 0.17 mmol), 2-tert-butyl-1,3-diisopropylisourea (200 mg, 1.0 mmol) and CH₂Cl₂ (4 mL) was stirred overnight at room temperature. The precipitate was removed by filtration and washed with CH₂Cl₂. The filtrate was concentrated, and the residue was purified by chromatography on silica gel using hexane/EtOAc (85:15) to give 7 as pale yellow oil (58 mg, 73%), ¹H NMR (500 MHz, CDCl₃) δ 8.64 (s, 1H), 8.49 (s, 1H), 7.95 (d, J=8.5 Hz, 1H), 7.68 (dd, J=7.0, 2.5 Hz, 1H), 7.39-7.30 (m, 2H), 7.21-7.18 (m, 1H), 7.15 (t, J=8.5 Hz, 1H), 6.99 (t, J=8.5 Hz, 1H), 5.80-5.72 (m, 1H), 4.99-4.90 (m, 2H), 4.68 (t, J=7.0 Hz, 1H), 2.60-2.53 (m, 1H), 2.14-2.06 (m, 3H), 1.69 (s, 9H), 1.51-1.30 (m, 2H).

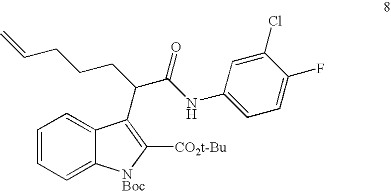

The same procedure as for 2 was employed for 8. ¹H NMR (500 MHz, CDCl₃) δ 8.83 (s, 1H), 7.88 (d, J=9.0 Hz, 1H), 7.74 (dd, J=7.0, 2.5 Hz, 1H), 7.40-7.36 (m, 1H), 7.32-7.29 (m, 1H), 7.27-7.24 (m, 1H), 6.99 (t, J=8.5 Hz, 1H), 5.76-5.68 (m, 1H), 4.98-4.90 (m, 2H), 3.98 (dd, J=8.5, 6.0 Hz, 1H), 2.38-2.31 (m, 1H), 2.19-1.99 (m, 3H), 1.70 (s, 9H), 1.64 (s, 9H), 1.36-1.27 (m, 2H).

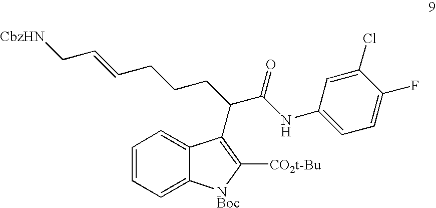

Compound 9 was obtained by a cross-metathesis reaction utilizing the procedure described by Biswas et al. (see: Tetrahedron Lett. 2002, 43, 6107) from a Cbz protected allylamine and 8. ¹H NMR (500 MHz, CDCl₃) δ 8.82 (s, 1H), 7.88 (d, J=8.5 Hz, 1H), 7.83 (d, J=8.0 Hz, 1H), 7.74 (dd, J=6:5, 2.5 Hz, 1H), 7.39-7.28 (br.m, 7H), 7.26-7.23 (m, 1H), 6.99 (t, J=8.5 Hz, 1H), 5.53-5.50 (m, 1H), 5.42-5.38 (m, 2H), 5.09 (s, 2H), 4.69 (br.s, 1H), 3.96 (dd, J=8.5, 6.0 Hz, 1H), 3.73-2.69 (m, 2H), 2.34-2.29 (m, 1H), 2.19-1.98 (m, 3H), 1.71 (s, 9H), 1.64 (s, 9H), 1.36-1.20 (m, 2H).

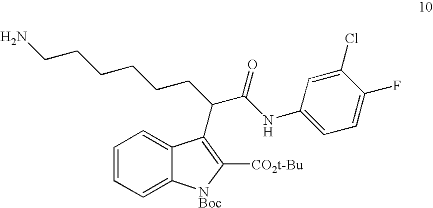

A mixture of 9 (74 mg, 0.1 mmol), Pd/C (5%, 37 mg) and methanol (6 mL) was stirred for 1 h under hydrogen (balloon) at room temperature. The catalyst was removed by filtration through Celite and washed with methanol. Removal of the solvent gave the amine as colorless oil, which was used directly for the next step without further purification.

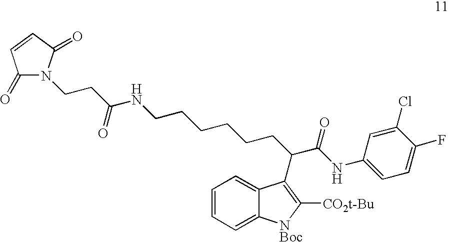

To the amine 10 (67 mg, 0.11 mmol) was added a solution of 3-maleimidopropionic acid (34 mg, 0.2 mmol), HBTU (68 mg, 0.18 mmol), and (i-Pr)₂EtN (0.07 mL, 0.4 mmol) in CH₂Cl₂ (3 mL). The mixture was stirred at room temperature for 2 h before quenched with 1 N HCl, and extracted with ethyl acetate. The organic layer was washed with saturated NaHCO₃ and brine, filtered and concentrated. The residue was purified by chromatography on silica gel using hexane/EtOAc (60:40 to 40:60) to afford 11 as colorless oil (60 mg, 72%). ¹H NMR (500 MHz, CDCl₃) δ 8.84 (s, 1H), 7.89 (d, J=8.5 Hz, 1H), 7.84 (d, J=8.5 Hz, 1H), 7.74 (dd, J=6.5, 2.5 Hz, 1H), 7.40-7.37 (m, 1H), 7.32-7.24 (m, 2H), 7.00 (t, J=9.0 Hz, 1H), 6.67 (s, 2H), 5.56-5.52 (m, 1H), 3.96 (dd, J=8.0, 7.0 Hz, 1H), 3.82 (t, J=7.0 Hz, 2H), 3.18-3.14 (m, 2H), 2.49 (t, J=7.0 Hz, 2H), 2.38-2.30 (m, 1H), 2.13-2.04 (m, 1H), 1.71 (s, 9H), 1.64 (s, 9H), 1.43-1.37 (m, 2H), 1.32-1.20 (m, 6H).

### Example 21

### Synthesis of Compound (Ia)-Tetrazole Derivative

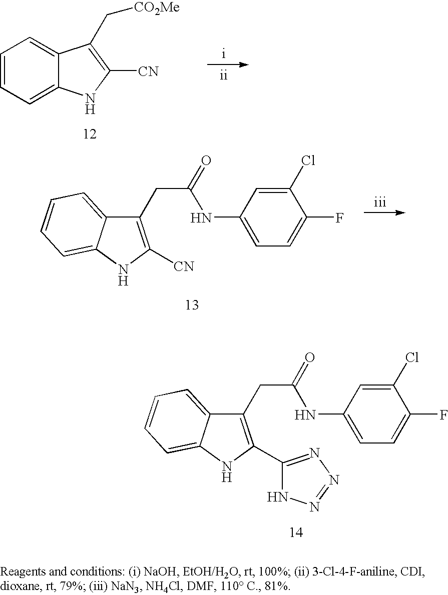

The ester 12 (214 mg, 1.0 mmol) prepared by the method described by Denison and Hilton (sec: Synlett, 2004, 15, 2806) was dissolved in ethanol (9 mL) and treated with 12 N NaOH (1 mL). The mixture was stirred at room temperature for 2 h before quenched with 1 N HCl, and extracted with ethyl acetate. The organic layer was washed with brine, dried over anhydrous Na₂SO₄, filtered, and concentrated to give a product (acid of 12) as pale yellow solid, which was used directly for the next step.

A mixture of the acid of 12 (0.6 mmol), 1,1′-carbonyldiimidazole (CDI, 98 mg, 0.6 mmol) and 1,4-dioxane (6 mL) was stirred for 3 hours at room temperature before adding 3-chloro-4-fluoroaniline (86 mg, 0.6 mmol). The reaction mixture was stirred for 2 d before quenched with 1 N HCl, and extracted with ethyl acetate. The organic solution was washed with brine, dried over anhydrous Na₂SO₄, filtered, and concentrated. The residue was purified by chromatography on silica gel using hexane/EtOAc (85:15→60:40) to give 13 as light yellow solid (155 mg, 79%). ¹H NMR (300 MHz, DMSO-d6) δ 12.25 (s, 1H), 10.56 (s, 1H), 7.92 (dd, J=6.9, 2.4 Hz, 1H), 7.40 (d, J=7.8 Hz, 1H), 7.52-7.32 (m, 4H), 7.16 (t, J=7.8 Hz, 1H), 3.96 (s, 2H).

A mixture of 13 (65 mg, 0.2 mmol), NaN₃ (65 mg, 1.0 mmol), NH₄Cl (53 mg, 1.0 mmol) and DMF (3 mL) was heated to 110° C. and stirred overnight and then allowed to cool to room temperature, quenched with 1 N HCl, and extracted with ethyl acetate. The organic layer was washed with brine, dried over anhydrous Na₂SO₄, filtered, and concentrated. The residue was purified by chromatography on silica gel using CH₂Cl₂/MeOH (9:1) to give 14 as white solid (60 mg, 81%). ¹H NMR (500 MHz, DMSO-d6) δ 11.68 (s, 1H), 11.21 (s, 1H), 7.87 (dd, J=7.0, 3.0 Hz, 1H), 7.66 (d, J=7.5 Hz, 1H), 7.46-7.42 (m, 2H), 7.34 (t, J=9.0 Hz, 1H), 7.18 (t, J=7.5 Hz, 1H), 7.34 (t, J=7.5 Hz, 1H), 4.12 (s, 2H).

### Example 22

### Lack of Toxicity of Compound (Ia) in Mice

An important aspect of the medicinal chemistry effort is a thorough analysis of potential in vivo toxicity. To determine whether Compound (Ia) exhibits acute toxicity in mice we injected two mice with a 9 mg/kg dose subcutaneously and observed mice over a 24 hour period. The mice remained calm and exhibited no sips of distress.

### Example 23

### Effect of Compound (Ia) on Peptide Presentation by Dendritic Cells In Vivo

Applicants next established the higher levels of peptide presentation in the presence of Compound (Ia) in human antigen presenting cells (APCs). The goal of these experiments is to examine whether Compound (Ia) covalently linked to the MBP peptide (abbreviated as Compound (Ia)*MBP peptide) and Compound (Ia) co-administered with peptide (Compound (Ia)+MBP peptide) enhance binding to MHC-II in vivo compared to unmodified peptide. The MK16 mAb (Krogsgaard et al., 2000, J. Exp. Med., 191:1395-1412) is used to directly quantify DR2/MBP peptide complexes on the surface of dendritic cells in lymph nodes draining the s.c. injection site. Lymph nodes are dissected 24 hours following injection of peptides in PBS and cell suspensions are labeled with CD11c (dendritic cells), MK16 (DR2/MBP), and Annexin-V (exclusion of apoptotic cells) for FACS analysis. The efficacy of Compound (Ia)*MBP peptide and unmodified MBP peptide are compared at concentrations ranging from 1 to 100 μM administered s.c. in the flank in 0.2 ml of PBS. In the co-administration mode, Compound (Ia) is added to the inoculum at concentrations of 50-200 μM. Compound (Ia) increases loading of peptides in a dose-dependent manner.

### Example 24

### In Vivo Evaluation of Compound (Ia)-Catalyzed Loading of Tolerogenic Peptide

Studies in several animal models have demonstrated that administration of soluble peptides can be used to inhibit or treat T cell mediated autoimmunity, due to induction of Th2 cells or T_{reg} specific for the peptide of interest (Kennedy et al., 1990, J. Immunol., 144:909-915; Gaur et al., 1992, Science, 258:1491-1494; Critchfield et al., 1994, Science 263:1139-43; Kohm et al., 2005, Int. Rev. Immunol., 24:361-392). In these treatment experiments it is examine whether more profound tolerance can be induced at lower peptide concentrations in the presence of Compound (Ia). The efficacy of Compound (Ia) is assessed in two settings, inhibition of disease induced by immunization of DR2/TCR transgenic mice with MBP peptide in complete Freund's adjuvant (CFA) and inhibition of spontaneous disease in DR2/TCR transgenic mice on a Rag2−/− background. In the first set of experiments, T cell tolerance is induced by injection of unmodified MBP peptide, MBP 92D control peptide (that does not bind to DR2), Compound (Ia)*MBP peptide, Compound (Ia)+MBP peptide s.c. in PBS (50-500 μg of peptide in 0.2 ml PBS, Compound (Ia) at 50-200 μM in coadministration mode), and EAE is induced by immunization with MBP peptide in CFA (150 μg of MBP peptide in CFA s.c. in a volume of 0.2 ml in the inguinal and axillary areas) (Madsen et al., 1999, Nat. Genet., 23:343-347). The development of EAE is assessed clinically on a 1 to 5 scale (1—limp tail, 2—hind limb weakness, 3—hind limb paralysis, 4—hindlimb and forelimb paralysis, 5—moribund or death from EAE). For histological analysis, brain and spinal cord tissue are fixed in 4% paraformaldehyde, embedded in paraffin and 8 μm sections stained with Luxol fast blue hematoxylin-eosin. Parenchymal inflammatory foci (20 or more inflammatory cells) are counted in standard cerebrum, midbrain, brainstem/cerebellum and upper and lower spinal cord sections. In addition, each of these inflammatory foci is assessed for demyelination, as judged by loss of blue staining in the area immediately around the lesion. Histological analysis is done by a single observer without knowledge of the treatment the animals received (Sobel et al., 1984, J. Immunol., 132:2393-2401; Madsen et al., 1999, Nat. Genet., 23:343-347).

Following these MBP/CFA immunization experiments, the efficacy of Compound (Ia)*MBP peptide and Compound (Ia)-linked MBP are compared to MBP peptide in the inhibition of spontaneous disease. Disease in these Rag2^{-/-} transgenic mice is aggressive and this model is thus a good test for addressing the question of whether use of Compound (Ia) permits induction of a more profound degree of T cell tolerance. DR2/TCR transgenic mice on the Rag2^{-/-} background are treated with Compound (Ia)*MBP peptide, Compound (Ia)+MBP peptide, MBP peptide or MBP 92D negative control peptide (50-500 μg of peptide in 0.2 ml PBS, Compound (Ia) at 50-200 μM in co-administration mode) at three weeks of age before the animals develop signs of spontaneous disease and then followed over an extended time until all mice in the control group have developed spontaneous disease (˜16 weeks of age). At the end of the observation period, quantitative histological analysis of brain and spinal cord is performed as described above.

### Example 25

### DR Inhibitors

The ability of the novel compounds to inhibit antigen presentation to MBP specific T cell clones isolated from MS patients is evaluated. These T cell clones are specific for the immunodominant MBP (85-99) peptide bound to DR2 or DQ1 (clones Ob.1A12, Hy.2E11, and Hy.1B11) and have been previously characterized in great detail (Wucherpfennig et al., 1994, J. Exp. Med., 179:279-290; Wucherpfennig and Strominger, 1995, Cell, 80:695-705). Peptide presentation is assessed based on the level of T cell proliferation and cytokine production using [³H]-thymidine incorporation and γ-interferon secretion assays, respectively. A DR2 homozygous EBV transformed B cell line (MGAR) that efficiently presents MBP is used as APC to present the MBP peptide to these cell clones. Because dendritic cells represent critical APC in vivo, the experiments are performed with human dendritic cells differentiated from blood monocytes using GM-CSF and IL-4 (Sallusto and Lanzavecchia, 1994, J. Exp. Med., 179:1109-18). B cells or dendritic cells are pulsed with rMBP (100 nM, expressed in E. coli) in the presence of unmodified Compound (Ia) or Compound (Ia)-linked PV-036 inhibitor over a wide range of inhibitor concentrations, washed, irradiated and co-cultured with T cells (5×10⁴ APC and 5×10⁴ T cells per well of a 96-well plate in triplicates). Supernatants are collected for cytokine measurements at 48 hours and [³H]-thymidine incorporation is assessed at 72 hours.

### Example 26

### Increasing the Efficacy of Glatiramer Acetate Using Compound (Ia)

Compound (Ia) either co-administered or conjugated to glatiramer acetate can enhance the loading of glatiramer acetate onto DR molecules of dendritic cells at the local skin injection site.

For covalent attachment, the ε-amino group of L-lysine, one of the four amino acids present in glatiramer acetate, is targeted. A Compound (Ia)-succinimide ester derivative for attachment to L-lysine is synthesized in a variation of the synthesis scheme already utilized for the creation of the Compound (Ia)-maleimide that binds to cysteines (we first generated the maleimide derivative because it only binds to cysteines while the succinimide ester will bind to both the α-amino group at the N-terminus of a peptide and the ε-amino group of L-lysine, unless the α-amino group is protected). The Compound (Ia)-succinimide ester is allowed to react with glatiramer acetate and then free Compound (Ia) is removed by dialysis using a membrane with a 2 kDa cutoff. As an alternative, the solid phase synthesis approach is used, as 50-mers generated by solid phase synthesis with the same amino acid ratios have comparable activity to glatiramer acetate in animal models. This synthetic approach permits a defined number of Compound (Ia) groups to be introduced at specified positions using the Compound (Ia)-maleimide derivative described above.

Compound (Ia)-glatiramer acetate compounds are characterized for their ability to compete for binding of the Alexe™-488 labeled MBP peptide to DR2 in the FP assay, as well as for their ability to inhibit presentation of the MBP peptide to human MBP specific T cell clones, as described in detail above. The Compound (Ia)-glatiramer acetate derivatives show more binding competition activity than glatiramer acetate alone.

It is then determined whether Compound (Ia) co-administered with glatiramer acetate or Compound (Ia)-linked glatiramer acetate increases the efficacy of glatiramer acetate in the DR2/TCR transgenic mouse model (Madsen et al., 1999, Nat. Genet., 23:343-347; Stern et al., 2004, Proc. Natl. Acad. Sci. USA, 101:11743-48). Treatment is initiated when the mice have developed mild EAE (day 9-10 in DR2/TCR transgenic mice). Glatiramer acetate, Compound (Ia)*glatiramer acetate and Compound (Ia)+glatiramer acetate are administered daily for 5 days subcutaneously in PBS (50-150 μg of glatiramer acetate per injection, together with different quantities of Compound (Ia) in the co-administration mode). Glatiramer acetate reduces the severity of EAE [i.e., from a mean score of ˜4 to a score of ˜2 in DR2/TCR transgenic mice and other EAE models (Illés et al., 2004, Proc. Natl. Acad. Sci. USA, 101:11749-54; Stern et al., 2004, Proc. Natl. Acad. Sci. USA, 101:11743-48)] but does not completely suppress the disease. The coadministration of Compound (Ia) and glatiramer acetate is superior to glatiramer acetate alone in reducing EAE symptoms, Likewise, coadministration of the Compound (Ia)-linked glatiramer acetate is superior to glatiramer acetate alone in reducing EAE symptoms in this model.

### Example 27

### Cytokine Display on Antigen Presenting Cells

Cytokines are will be expressed either in E. coli (β-interferon and IL-10) or CHO cells (TGFβ) as fusion proteins with N-terminal or C-terminal peptides that bind with high affinity to DR molecules. Compound (Ia) is covalently attached to a free cysteine residue on the C-terminus of the peptide with a maleimide derivative. Display of these cytokines via DR molecules on the surface of human APC is examined. These peptide-cytokine fusion proteins are incubated with human EBV transformed B cells, and the level of surface display is quantitated by using an antibody directed against an epitope tag attached to the cytokine in the presence and absence of different concentrations of Compound (Ia) and related compounds. The efficiency of cytokine surface display is compared when the Compound (Ia) group is covalently linked to the cytokine-peptide fusion protein or when it is co-administered.

The cytokine-peptide fusion protein and Compound (Ia) are tested promotion of the differentiation and/or expansion of self-reactive T cells towards a regulatory phenotype, e.g., the IL-10 producing Tr1 cells (driven by IL-10), which are known to have protective properties in animal models of chronic inflammation and asthma, and Foxp3⁺ Treg (driven by TGFβ1), which is known to offer protection from autoimmunity in a variety of animal models. B cells or dendritic cells from DR2 transgenic mice (a humanized mouse model of MS, developed by transgenic expression of HLA-DR2 and a TCR from a patient with relapsing-remitting MS; referred to as DR2/hTCR transgenic mice) are incubated with the peptide-cytokine fusion proteins. The cells are co-cultured with naïve T cells from DR2/hTCR transgenic mice in the presence of the MBP peptide recognized by the TCR. The cytokine profile of these T cell populations is determined by intracellular cytokine staining. In addition, cytokine production in supernatants is measured following stimulation with APC plus peptide in an ELISA (IL-10, IL-4, γ-interferon as markers of Tr1, Th2 and Th1 cells, respectively). Differentiation into Foxp3⁺ Treg is determined by intracellular staining for Foxp3. When differentiation towards potentially protective phenotypes (i.e., IL-10 producing Tr1 phenotype or Foxp3+ phenotype) is identified, the protective properties of these T cells is determined by passive transfer into DR2/hTCR transgenic mice, either immediately following immunization of mice with the MBP peptide or coincident with early signs of EAE or at the peak of disease.

The therapeutic efficacy of peptide-cytokine fusion proteins is evaluated in the humanized mouse model of MS described above. This humanized mouse model provides an opportunity to test compounds on relevant human targets. The in vivo display of peptide-cytokine fusion proteins on different APC populations (B cells, dendritic cells) is quantitated by FACS based on an epitope tag attached to the cytokine following s.c. or i.v. administration into DR2/hTCR transgenic mice. These experiments are used to establish the dose range for both peptide-cytokine fusion proteins and Compound (Ia) in efficacy studies. The efficacy of compounds is tested, first, by immunization with MBP (85-99) peptide in adjuvant (complete Freund's adjuvant, CFA) inducing disease within ˜10 days and, second, by an assessment of spontaneous disease (incidence of ˜60%). Measurements of efficacy include neurological scores and histology to assess inflammation and CNS demyelination.

### Example 28

### Toxicology and Pharmacokinetics

Acute maximum tolerated dose (MTD) studies are used for chronic dose ranging studies. Three groups of mice (n=6) are dosed at MTD/3, MTD/10 and MTD/30 daily for 10 days. A dose is judged non-toxic and suitable for pharmacokinetics and efficacy studies based upon an assessment of the animal's general health, behavior and weight loss. Brains are harvested and the compound's ability to cross the blood brain barrier assessed, both in normal mice and mice with EAE, because the blood brain barrier is permeable even to large proteins at sites of inflammation. After dosing, T cell populations are tested to determine the relative ratios of T cells (CD4/CD8 T cells) and other white blood cells in lymph nodes, spleen, and peripheral blood. Organs will be checked for aberrant lymphocyte infiltrations. Compounds are administered either i.v. or i.p. to mice at a dose of 10 mg/kg or a dose determined by animal toxicity studies outlined above. Multiple samples of blond are collected over a period of 24 hours and analyzed for the parent compound. With the plasma half-life, a bioavailability calculation is used to determine the dosing for animal efficacy studies described above.

### Other Embodiments

A number of embodiments of the invention have been described. Nevertheless, it will be understood that various modifications can be made without departing from the spirit and scope of the invention. Accordingly, other embodiments are within the scope of the following claims.

1. A pharmaceutical composition comprising a pharmaceutically acceptable carrier or diluent and a compound represented by Structural Formula (I): 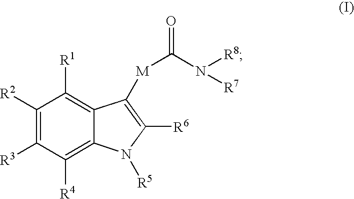
or a pharmaceutically acceptable salt thereof, wherein:
R¹, R², R³, and R⁴ are each independently selected from —H, —Cl, —F, —CH₃, —Br, —CF₃, —OCF₃, —CN, —CO₂R*, —OR*, —NR*R*, —SO₂R*, and —SO₂NR*R*;
R* in each occurrence is independently selected from H and substituted or unsubstituted alkyl, aryl, and alkenyl;
R⁵ is —H, -lower alkyl, or lower alkenyl;
R⁶ is —CO₂H, —CO₂R′, —SO₃H, SO₃R′ or 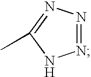
R′ is lower alkyl;
R⁷ is aromatic, aliphatic, or alkyl interrupted by one or more heteroatoms;
R⁸ is —H or —CH₃ and
M is a covalent bond or can independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**, and —(CR**R**)ₙ—, and R^{**} independently for each occurrence, is H or lower alkyl;
n is 0-5; and
n′ is 0-2.

2-10. (canceled)

11. The pharmaceutical composition of claim 1, wherein the compound is represented by Structural Formula (Ia):

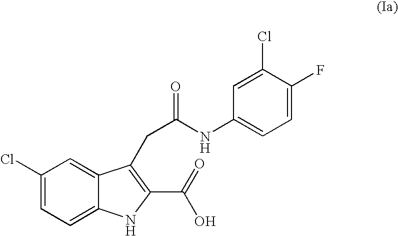

12. The pharmaceutical composition of claim 1, wherein the compound is represented by Structural Formula (Ib):

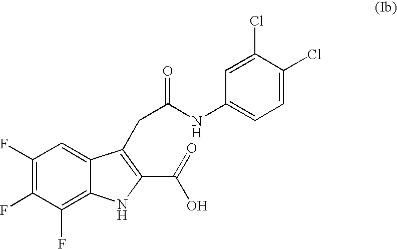

13. The pharmaceutical composition of claim 1, wherein the compound is represented by Structural Formula (Ic):

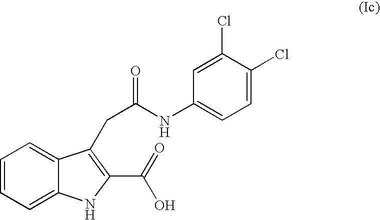

14. The pharmaceutical composition of claim 1, wherein the compound is represented by Structural Formula (Id):

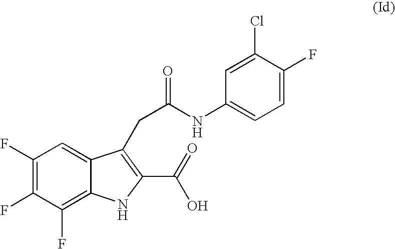

15-18. (canceled)

19. A composition of claim 1, further comprising a peptide that loads onto MHC Class II molecules.

20. A pharmaceutical composition comprising a pharmaceutically acceptable carrier or diluent and a compound represented by Structural Formula (IV): 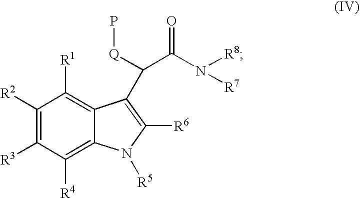
or a pharmaceutically acceptable salt thereof, wherein:
R¹, R², R³, and R⁴ are each independently selected from —H, —Cl, —F, —CH₃, and —OCH₃;
R⁵ is —H, —CH₃, lower alkyl or —(CH₂)₅CH═CH₂;
R⁶ is —CO₂H, —CO₂R′; —SO₃H, aliphatic, or aromatic;
R′ is lower alkyl;
R⁷ is aromatic, aliphatic, or alkyl interrupted by one or more heteroatoms;
R⁸ is —H or —CH₃;
Q is a covalent bond, an inert linking group, or a substituted inert linking group; and
P is a peptide that loads onto an MHC Class II molecule.

21-37. (canceled)

38. The pharmaceutical composition of claim 20, wherein the peptide binds to an MHC-class II molecule.

39. The pharmaceutical composition of claim 20, wherein the peptide is a copolymer.

40-41. (canceled)

42. The pharmaceutical composition of claim 20, wherein the peptide is an antigen.

43-46. (canceled)

47. The pharmaceutical composition of claim 20, wherein Q is M, wherein
M is a covalent bond or may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR**, O or S), —OC(O)—, —C(═O)O, —NR**—, —NR**CO—, —C(O)NR**—, —S(O)_{n′}—, —OC(O)—NR**, —NR**—C(O)—NR**—, —NR**—C(NR**)—NR**—, and —(CR**R**)ₙ— and R** independently for each occurrence, is H or lower alkyl; and
wherein the compound has a formula selected from:

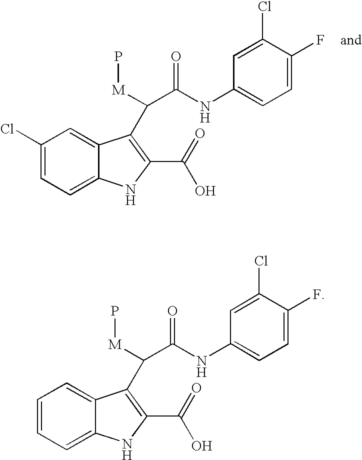

48-65. (canceled)

66. A method of increasing a rate of peptide exchange in MHC Class II molecules in a subject in need thereof, the method comprising administering to the subject a therapeutically-effective amount of the pharmaceutical composition of claim 1.

67. (canceled)

68. The method of claim 66, wherein the MHC Class II molecule is HLA-DR2.

69. The method of claim 66, wherein, the subject is afflicted with an autoimmune disorder.

70. (canceled)

71. The method of claim 69, wherein the autoimmune disorder is multiple sclerosis.

72-111. (canceled)

112. A method of increasing the rate of peptide exchange in MHC Class II molecules in a subject in need thereof, the method comprising administering to the subject a therapeutically effective amount of the pharmaceutical composition of claim 20.

113. The method of claim 112, wherein the MHC Class II molecule is HLA-DR2.

114. The method of claim 112, wherein, the subject is afflicted with an autoimmune disorder.

115. The method of claim 114, wherein the autoimmune disorder is multiple sclerosis.
